(19) United States
(12) Patent Application Publication
(10) Pub. No.: US 2024/0011010 A1
(43) Pub. Date: Jan. 11, 2024

(54) SYNTHETIC PROTEIN-LEVEL NEURAL NETWORK IN MAMMALIAN CELLS

(71) Applicant: California Institute of Technology, Pasadena, CA (US)

(72) Inventors: Zibo Chen, Pasadena, CA (US); Michael B. Elowitz, Pasadena, CA (US)

(21) Appl. No.: 18/347,423

(22) Filed: Jul. 5, 2023

Related U.S. Application Data
(60) Provisional application No. 63/358,607, filed on Jul. 6, 2022.

(51) Int. Cl. C12N 9/50; C12N 9/06; C07K 14/00

(52) U.S. Cl. CPC C12N 9/506; C12Y 304/22044; C12N 9/003; C12Y 105/01003; C07K 14/001

(57) ABSTRACT

Disclosed herein include methods, compositions, and kits suitable for use in winner-take-all neural network computation in mammalian cells. In some embodiments, de novo designed protein heterodimers and engineered viral proteases are combined to implement a synthetic protein circuit that performs winner-take-all neural network computation. The synthetic protein circuit can include modules that compute weighted sums of input protein concentrations through reversible binding interactions, and allow for self-activation and mutual inhibition of protein components using irreversible proteolytic cleavage reactions.

### RELATED APPLICATIONS

This application claims the benefit under 35 U.S.C. § 119(e) of U.S. Provisional Patent Application Ser. No. 63/358,607, filed Jul. 6, 2022, the content of this related application is incorporated herein by reference in its entirety for all purposes.

### STATEMENT REGARDING FEDERALLY SPONSORED R&D

This invention was made with government support under Grant No. MH116508 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365859-US, created Jul. 2, 2023, which is 28,745 bytes in size. The information in the electronic format of the Sequence Listing is incorporated herein by reference in its entirety.

### BACKGROUND

### Field

The present disclosure relates generally to the field of synthetic biology.

### Description of the Related Art

A foundational challenge in synthetic biology is classification, the ability to program a cell to respond in distinct ways to information encoded in multiple input signals. Classification is the basis for emerging diagnostic and therapeutic synthetic biology applications. Previous efforts at achieving synthetic classification circuits were either cell-free (DNA in test tubes) or limited to miRNA-level regulation, which cannot directly interact with the cellular proteins that encode most cellular signals. While protein level classification circuits would allow more direct interactions with key cellular pathways, proteins are far more difficult to engineer. For this reason, a synthetic protein level classification circuit has been lacking. There is a need for compositions, systems, and methods for synthetic protein level classification circuits.

### SUMMARY

Disclosed herein include synthetic protein circuits. The synthetic protein circuit can comprise: a first n-node protein comprising a first part of a first protease domain, a first dimerization domain, and a second dimerization domain. The synthetic protein circuit can comprise: a companion first n-node protein comprising a first part of a first protease domain, a third dimerization domain, and a fourth dimerization domain. The synthetic protein circuit can comprise: a first c-node protein comprising a second part of a first protease domain and a fifth dimerization domain. The synthetic protein circuit can comprise: a second n-node protein comprising a first part of a second protease domain, a sixth dimerization domain, and a seventh dimerization domain. The synthetic protein circuit can comprise: a companion second n-node protein comprising a first part of a second protease domain, an eighth dimerization domain, and a ninth dimerization domain. The synthetic protein circuit can comprise: a second c-node protein comprising a second part of a second protease domain and a tenth dimerization domain. The synthetic protein circuit can comprise: a first input protein comprising an eleventh dimerization domain. The synthetic protein circuit can comprise: a second input protein comprising a twelfth dimerization domain.

In some embodiments, the first dimerization domain, the third dimerization domain, the sixth dimerization domain, and/or the eighth dimerization domain, are the same or have at least about 80% sequence identity. In some embodiments, the second dimerization domain and the seventh dimerization domain are the same or have at least about 80% sequence identity. In some embodiments, the fourth dimerization domain and the ninth dimerization domain are the same or have at least about 80% sequence identity. In some embodiments, the fifth dimerization domain and the tenth dimerization domain are the same or have at least about 80% sequence identity.

In some embodiments, one or more the first dimerization domain, the third dimerization domain, the sixth dimerization domain, and the eighth dimerization domain are capable of binding one or more of the second dimerization domain, the seventh dimerization, the fourth dimerization domain, the ninth dimerization domain, the fifth dimerization domain, and the tenth dimerization domain. In some embodiments, the eleventh dimerization domain is capable of binding the second dimerization domain and/or the seventh dimerization domain. In some embodiments, the twelfth dimerization domain is capable of binding the fourth dimerization domain and/or the ninth dimerization domain.

In some embodiments, intramolecular binding between the first dimerization domain and the second dimerization domain of the first n-node protein is capable of preventing the first n-node protein from binding the first c-node protein to form a first complex. In some embodiments, intramolecular binding between the first dimerization domain and the second dimerization domain of the first n-node protein is capable of preventing the first n-node protein from binding the second c-node protein to form a second complex. In some embodiments, intramolecular binding between the third dimerization domain and the fourth dimerization domain of the companion first n-node protein is capable of preventing the companion first n-node protein from binding the first c-node protein to form a third complex. In some embodiments, intramolecular binding between the third dimerization domain and the fourth dimerization domain of the companion first n-node protein is capable of preventing the companion first n-node protein from binding the second c-node protein to form a fourth complex. In some embodiments, intramolecular binding between the sixth dimerization domain and the seventh dimerization domain of the second n-node protein is capable of preventing the second n-node protein from binding the first c-node protein to form a fifth complex. In some embodiments, intramolecular binding between the sixth dimerization domain and the seventh dimerization domain of the second n-node protein is capable of preventing the second n-node protein from binding the second c-node protein to form a sixth complex. In some embodiments, intramolecular binding between the eighth dimerization domain and the ninth dimerization domain of the companion second n-node protein is capable of preventing the companion second n-node protein from binding the first c-node protein to form a seventh complex. In some embodiments, intramolecular binding between the eighth dimerization domain and the ninth dimerization domain of the companion second n-node protein is capable of preventing the companion second n-node protein from binding the second c-node protein to form an eighth complex.

In some embodiments, the eleventh dimerization domain of the first input protein is capable of disrupting intramolecular binding between the first dimerization domain and the second dimerization domain of the first n-node protein. In some embodiments, the twelfth dimerization domain of the second input protein is capable of disrupting intramolecular binding between the third dimerization domain and the fourth dimerization domain of the companion first n-node protein. In some embodiments, the eleventh dimerization domain of the first input protein is capable of disrupting intramolecular binding between the sixth dimerization domain and the seventh dimerization domain of the second n-node protein. In some embodiments, the twelfth dimerization domain of the second input protein is capable of disrupting intramolecular binding between the eighth dimerization domain and the ninth dimerization domain of the companion second n-node protein.

In some embodiments, intermolecular binding between the eleventh dimerization domain of the first input protein and the second dimerization domain of the first n-node protein enables the first dimerization domain of the first n-node protein to bind the fifth dimerization domain of the first c-node protein and thereby form a first complex. In some embodiments, intermolecular binding between the eleventh dimerization domain of the first input protein and the second dimerization domain of the first n-node protein enables the first dimerization domain of the first n-node protein to bind the tenth dimerization domain of the second c-node protein and thereby form a second complex. In some embodiments, intermolecular binding between the twelfth dimerization domain of the second input protein and the fourth dimerization domain of the companion first n-node protein enables the third dimerization domain of the companion first n-node protein to bind the fifth dimerization domain of the first c-node protein and thereby form a third complex. In some embodiments, intermolecular binding between the twelfth dimerization domain of the second input protein and the fourth dimerization domain of the companion first n-node protein enables the third dimerization domain of the companion first n-node protein to bind the tenth dimerization domain of the second c-node protein and thereby form a fourth complex. In some embodiments, intermolecular binding between the eleventh dimerization domain of the first input protein and the seventh dimerization domain of the second n-node protein enables sixth dimerization domain of the second n-node protein to bind the fifth dimerization domain of the first c-node protein and thereby form a fifth complex. In some embodiments, intermolecular binding between the eleventh dimerization domain of the first input protein and the seventh dimerization domain of the second n-node protein enables sixth dimerization domain of the second n-node protein to bind the tenth dimerization domain of the second c-node protein and thereby form a sixth complex. In some embodiments, intermolecular binding between the twelfth dimerization domain of the second input protein and the ninth dimerization domain of the companion second n-node protein enables the eighth dimerization domain of the companion second n-node protein to bind the fifth dimerization domain of the first c-node protein and thereby form a seventh complex. In some embodiments, intermolecular binding between the twelfth dimerization domain of the second input protein and the ninth dimerization domain of the companion second n-node protein enables the eighth dimerization domain of the companion second n-node protein to bind the tenth dimerization domain of the second c-node protein and thereby form an eighth complex.

In some embodiments, the first complex and/or the third complex comprise a first protease capable of being in a first protease active state. In some embodiments, the sixth complex and/or the eighth complex comprise a second protease capable of being in a second protease active state. In some embodiments, the second complex, fourth complex, fifth complex, and/or seventh complex do not comprise a first protease capable of being in a first protease active state or a second protease capable of being in a second protease active state. In some embodiments, the first part of the first protease domain and the second part of the first protease domain are capable of associating with each other to constitute a first protease capable of being in a first protease active state when: (i) the first n-node protein binds the first c-node protein to form a first complex; and/or (ii) the companion first n-node protein binds the first c-node protein to form a third complex. In some embodiments, the first part of the second protease domain and the second part of the second protease domain are capable of associating with each other to constitute a second protease capable of being in a second protease active state when: (i) the second n-node protein binds the second c-node protein to form a sixth complex; and/or (ii) the companion second n-node protein binds the second c-node protein to form an eighth complex. In some embodiments, the first n-node protein, the companion first n-node protein, the second n-node protein, and/or the companion second n-node protein comprise a degradation domain, optionally the presence of said degradation domain causes the protein to be a destabilized state.

In some embodiments, the first n-node protein and/or the companion first n-node protein comprise a first cut site the first protease in the first protease active state is capable of cutting, optionally the cleavage of said first cut site is capable of inactivating or removing the degradation domain, thereby causing the protein to be in stabilized state. In some embodiments, the second n-node protein and/or the companion second n-node protein comprise a second cut site the second protease in the second protease active state is capable of cutting, optionally the cleavage of said second cut site is capable of inactivating or removing the degradation domain, thereby causing the protein to be in stabilized state. In some embodiments, the first c-node protein comprises a second cut site the second protease in the second protease active state is capable of cutting, optionally the second cut site is situated between the second part of a first protease domain and the fifth dimerization domain, further optionally cleavage of the said second cut site prevents said first c-node protein from constituting a complex with active protease activity. In some embodiments, the second c-node protein comprises a first cut site the first protease in the first protease active state is capable of cutting, optionally the first cut site is situated between the second part of a second protease domain and the tenth dimerization domain, further optionally cleavage of the said first cut site prevents said second c-node protein from constituting a complex with active protease activity.

In some embodiments, first complexes and/or third complexes are capable of self-activation via first protease-mediated cleavage of the first cut site of first complexes and/or third complexes. In some embodiments, sixth complexes and/or eighth complexes are capable of self-activation via second protease-mediated cleavage of the second cut site of sixth complexes and/or eighth complexes. In some embodiments, first complexes and/or third complexes are capable of mutual inhibition via first protease-mediated cleavage of the first cut site of sixth complexes and/or eighth complexes. In some embodiments, sixth complexes and/or eighth complexes are capable of mutual inhibition via second protease-mediated cleavage of the first cut site of first complexes and/or third complexes.

In some embodiments, the degradation domain comprises a degron, optionally the degron comprises an N-degron, a dihydrofolate reductase (DHFR) degron, a FKB protein (FKBP) degron, derivatives thereof, or any combination thereof. In some embodiments, the first protease and/or the second protease comprises a viral protease. In some embodiments, the first protease and/or the second protease comprises tobacco etch virus (TEV) protease, tobacco vein mottling virus (TVMV) protease, hepatitis C virus protease (HCVP), derivatives thereof, or any combination thereof. In some embodiments, the first n-node protein, the companion first n-node protein, the first c-node protein, the second n-node protein, the companion second n-node protein, and/or the second c-node protein comprises a linker, optionally the linker is: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more.

In some embodiments, one or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, one or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, one or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain is a homodimerization domain or a multimerization domain, optionally a homo- or hetero-dimerizing or multimerizing leucine zipper, a PDZ domains, a SH3 domain, aGBD domain, or any combination thereof.

In some embodiments, the first n-node protein, the companion first n-node protein, and/or the first c-node protein constitute a first node of the synthetic protein circuit having first protease activity, and wherein the second n-node protein, the companion second n-node protein, and/or the second c-node protein constitute a second node of the synthetic protein circuit having second protease activity, optionally the synthetic protein circuit activates a single node. In some embodiments, the synthetic protein circuit further comprises m supplemental input proteins and/or n supplemental nodes, wherein n or m is an integer greater than zero. In some embodiments, the synthetic protein circuit does not comprise the companion first n-node protein and/or the second n-node protein. In some embodiments, the synthetic protein circuit achieves winner-take-all dynamics with tunable decision boundaries, wherein winner-take-all dynamics comprises a single protease activated in the synthetic protein circuit. In some embodiments, the output of the synthetic protein circuit depends on (i) the relative levels of the first input protein and the second input protein and (ii) the tunable decision boundaries of the synthetic protein circuit. In some embodiments, the output of the synthetic protein circuit is either the first protease in the first protease active state or the second protease in the second protease active state.

In some embodiments, the first protease in the first protease active state and/or the second protease in the second protease active state is capable of modulating the expression, localization, and/or stability of one or more input proteins of a downstream synthetic protein circuit. In some embodiments, the relative levels of the first input protein and/or the second input protein is modulated by an upstream synthetic protein circuit. In some embodiments, the relative levels of the first input protein and/or the second input protein are capable of being regulated via one or more of expression, localization, and stability. In some embodiments, the synthetic protein circuit is capable of winner-take-all classification of biological information, wherein said biological information is associated with the relative levels of the first input protein and the second input protein, further optionally said biological information relates to the presence and/or amount of a unique cell type and/or a unique cell state in a cell. In some embodiments, the relative levels of the first input protein and the second input protein is correlated with the presence and/or amount of a unique cell type and/or a unique cell state in a cell. In some embodiments, the degree of expression and/or degradation of the first input protein and/or the second input protein is associated with the presence and/or amount of a unique cell type and/or a unique cell state. In some embodiments, an input protein comprises a degradation domain and a cut site, wherein a protease is capable of cutting the cut site of the input protein to hide the degradation domain, and wherein the degradation domain of the input protein being hidden changes the input protein to an input protein stabilized state. In some embodiments, the input protein comprises a degradation domain and a cut site, wherein a protease is capable of cutting the cut site of the input protein to expose the degradation domain, and wherein the degradation domain of the input protein being exposed changes the input protein to an input protein destabilized state.

The synthetic protein circuit can comprise: one or more modulator circuit proteins configured to regulate the expression and/or stability of the first input protein and/or the second input protein in response to the cell type and/or cell state of a cell. In some embodiments, the tunable decision boundaries are capable of being tuned by adjusting the relative levels of the first n-node protein, the companion first n-node protein, the first c-node protein, the second n-node protein, the companion second n-node protein, and/or the second c-node protein, optionally said adjusting comprises differential expression. In some embodiments, the tunable decision boundaries are capable of being tuned by introducing one or more amino acid substitutions into the degradation domains(s), the cut site(s) and/or the dimerization domains(s). In some embodiments, the synthetic protein circuit is present in a cell. In some embodiments, the cell is: a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, or an in situ cell. In some embodiments, the cell is a eukaryotic cell, optionally a mammalian cell, further optionally the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

In some embodiments, a synthetic protein circuit is capable of modulating the expression, concentration, localization, stability, and/or activity of the one or more exogenous payload protein(s) and/or one or more endogenous payload protein(s) of a cell. In some embodiments, the first protease in the first protease active state or the second protease in the second protease active state is capable of modulating the expression, concentration, localization, stability, and/or activity of the one or more exogenous payload protein(s) and/or one or more endogenous payload protein(s) of a cell In some embodiments, the first protease in the first protease active state or the second protease in the second protease active state is capable of modulating the cell type and/or cell state of a cell based on the presence and/or amount of a unique cell type and/or a unique cell state in a cell. In some embodiments, the synthetic protein circuit further comprises one or more effector proteins comprising a first cut site the first protease in the first protease active state is capable of cutting or a second cut site the second protease in the second protease active state is capable of cutting, thereby modulating its expression, concentration, localization, stability, and/or activity, optionally said one or more effector proteins are capable of modulating the expression, concentration, localization, stability, and/or activity of the one or more exogenous payload protein(s) and/or one or more endogenous payload protein(s) of a cell. In some embodiments, the synthetic protein circuit is configured to be responsive to changes in: cell environment, optionally cell environment comprises location relative to a target site of a subject and/or changes in the presence and/or absence of cell(s) of interest, optionally said cell(s) of interest comprise target-specific antigen(s); one or more signal transduction pathways regulating cell survival, cell growth, cell proliferation, cell adhesion, cell migration, cell metabolism, cell morphology, cell differentiation, apoptosis, or any combination thereof input(s) of a synthetic receptor system, optionally Synthetic Notch (SynNotch) receptor, a Modular Extracellular Sensor Architecture (MESA) receptor, a synthekine, Tango, dCas9-synR, a chimeric antigen receptor, or any combination thereof; and/or T cell activity, optionally T cell activity comprises one or more of T cell simulation, T cell activation, cytokine secretion, T cell survival, T cell proliferation, CTL activity, T cell degranulation, and T cell differentiation.

In some embodiments, the synthetic protein circuit is: (i) capable of modulating cell states, cell types, and/or cell behaviors, (ii) configured to selectively activate cell death and/or immune recruitment to tumor cells; and/or (iii) is configured to detect the intracellular state of a cell and classify it as tumor or normal based on the levels or activities of relevant molecules or pathways. In some embodiments, a unique cell type and/or a unique cell state comprises a unique gene expression pattern, optionally the unique cell type and/or unique cell state comprises a unique anatomic location, further optionally the unique cell type and/or the unique cell state comprises anatomically locally unique gene expression.

In some embodiments, a unique cell type and/or a unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment, optionally the senescent cell state induced by a tumor microenvironment comprises expression of CD57, KRLG1, TIGIT, or any combination thereof. In some embodiments, the unique cell state and/or unique cell type is characterized by aberrant signaling of one or more signal transducer(s). In some embodiments, the unique cell state comprises: a physiological state, optionally a cell cycle state, a differentiation state, a development state a metabolic state, or a combination thereof; and/or a pathological state, optionally a disease state, a human disease state, a diabetic state, an immune disorder state, a neurodegenerative disorder state, an oncogenic state, or a combination thereof. In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

In some embodiments, the aberrant signaling involves an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. In some embodiments, the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof, optionally the signal transducer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6, or any combination thereof, optionally the disease or disorder is characterized by an aberrant signaling of the first transducer.

In some embodiments, a payload protein is capable of modulating the expression, concentration, localization, stability, and/or activity of the one or more endogenous proteins of a cell. In some embodiments, the payload protein is a therapeutic protein or a variant thereof, optionally a therapeutic protein configured to prevent or treat a disease or disorder of a subject, further optionally the subject suffers from a deficiency of said therapeutic protein. In some embodiments, a payload protein comprises: fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof; nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof; a cellular reprogramming factor capable of differentiating a given cell into a desired differentiated state, optionally nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof; an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a secretion tag, optionally the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC; a constitutive signal peptide for protein degradation, optionally PEST; a nuclear localization signal (NLS) or a nuclear export signal (NES); a dosage indicator protein, optionally the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof; a cellular reprogramming factor capable of converting an at least partially differentiated cell to a less differentiated cell, optionally Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof; a programmable nuclease, optionally the synthetic protein circuit senses correction of an aberrant locus by said programmable nuclease and reduces effector protein localization and/or activity, optionally the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions; a CRE recombinase, GCaMP, a cell therapy component, a knock-down gene therapy component, a cell-surface exposed epitope, or any combination thereof; a bispecific T cell engager (BiTE); a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof; a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5; a member of the TNF family of cytokines selected from the group consisting of TNF-alpha, TNF-beta, LT-beta, CD40 ligand, Fas ligand, CD 27 ligand, CD 30 ligand, and 4-1 BBL; a member of the immunoglobulin superfamily of cytokines selected from the group consisting of B7.1 (CD80) and B7.2 (B70); an interferon, optionally the interferon is selected from interferon alpha, interferon beta, or interferon gamma; a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15; an interleukin, optionally the interleukin is selected from IL-10 IL-12, IL-1, IL-6, IL-7, IL-15, IL-2, IL-18 or IL-21; a tumor necrosis factor (TNF), optionally the TNF is selected from TNF-alpha, TNF-beta, TNF-gamma, CD252, CD154, CD178, CD70, CD153, or 4-1BBL; a factor locally down-regulating the activity of endogenous immune cells; is capable of remodeling a tumor microenvironment and/or reducing immunosuppression at a target site of a subject; a chimeric antigen receptor (CAR) or T-cell receptor (TCR); and/or an activity regulator, optionally the activity regulator is capable of reducing T cell activity, optionally the CAR and/or TCR comprises one or more of an antigen binding domain, a transmembrane domain, and an intracellular signaling domain, optionally wherein the intracellular signaling domain comprises a primary signaling domain, a costimulatory domain, or both of a primary signaling domain and a costimulatory domain.

In some embodiments, the activity regulator: comprises a ubiquitin ligase involved in TCR/CAR signal transduction selected from the group comprising c-CBL, CBL-B, ITCH, R F125, R F128, WWP2, or any combination thereof; comprises a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof; is a negative regulatory scaffold/adapter protein selected from the group comprising PAG, LIME, NTAL, LAX31, SIT, GAB2, GRAP, ALX, SLAP, SLAP2, DOK1, DOK2, or any combination thereof; is a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAP1, CRKII, C3G, WAVE2, ARP2/3, ABL, ADAP, RIAM, SKAP55, or any combination thereof; comprises the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof; is targeted to the plasma membrane with a targeting sequence derived from LAT, PAG, LCK, FYN, LAX, CD2, CD3, CD4, CD5, CD7, CD8a, PD1, SRC, LYN, or any combination thereof; and/or reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

In some embodiments, a payload protein is an activity regulator, optionally the activity regulator is capable of reducing T cell activity. In some embodiments, the payload protein comprises a pro-death protein capable of halting cell growth and/or inducing cell death; wherein the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof; and/or wherein the pro-death protein is capable of halting cell growth and/or inducing cell death in the presence of a pro-death agent. In some embodiments: the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-α,γ-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU).

In some embodiments, a payload protein comprises one or more receptors and/or a targeting moiety configured to bind a component of a target site of a subject, optionally the target site is a site of disease or disorder or is proximate to a site of a disease or disorder. In some embodiments, the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the one or more targeting moieties are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-I, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-y, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αιξβ3, α?β3, α4β7, α5β1, α6β4, α7β7, α11β3, α5β5, ανβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-βI, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

Disclosed herein include nucleic acid compositions. The nucleic acid composition can comprise: one or more polynucleotides encoding a synthetic protein circuit provided herein (or components thereof). In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the one or more polynucleotides are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m 1Ψ), and 5-methyl-uridine (m5U). In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors, optionally at least one of the one or more vectors is a viral vector, a plasmid, a transposable element, a naked DNA vector, a lipid nanoparticle (LNP), or any combination thereof, optionally the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and optionally the transposable element is piggybac transposon or sleeping beauty transposon. In some embodiments, the one or more polynucleotides are comprised in the one or more vectors, optionally the one or more polynucleotides are comprised in the same vector and/or different vectors, optionally the one or more polynucleotides are situated on the same nucleic acid and/or different nucleic acids. In some embodiments, the nucleic acid composition is configured to achieve relative levels of the first input protein and/or second input protein dependent on a unique cell type and/or unique cell state. In some embodiments, the nucleic acid composition is configured to achieve relative levels of the first n-node protein, the companion first n-node protein, the first c-node protein, the second n-node protein, the companion second n-node protein, and/or the second c-node protein desired by a user. Disclosed herein include compositions (e.g., pharmaceutical compositions) comprising a nucleic acid composition provided herein.

Disclosed herein include systems for classifying multi-input signals in a cell, comprising one or more components of the synthetic protein circuits provided herein. Disclosed herein include systems for classifying the cell type and/or cell state of a cell, comprising one or more components of the synthetic protein circuits provided herein. Disclosed herein include engineered cells or a population of engineered cells, comprising: a synthetic protein circuit provided herein, one or more components of the synthetic protein circuits provided herein and/or the nucleic acid compositions provided herein. Disclosed herein include methods for classifying the cell type and/or cell state of a cell, comprising: expressing a synthetic protein circuit provided herein or one or more components of the synthetic protein circuits provided herein in the cell. Disclosed herein include methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

In some embodiments, administering comprises: (i) isolating one or more cells from the subject; (ii) contacting said one or more cells with a nucleic acid composition provided herein, thereby generating engineered cells, optionally the contacting comprises transfection; and (iii) administering the one or more engineered cells into a subject after the contacting step. In some embodiments, the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, optionally a solid tumor. In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enterovirus (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen, optionally the disease associated with expression of a tumor antigen-associated is selected from the group consisting of a proliferative disease, a precancerous condition, a cancer, and a non-cancer related indication associated with expression of the tumor antigen. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

FIGS. 1A-1H depict non-limiting exemplary schematics showing how winner-take-all neural network computation can be implemented using engineered proteins provided herein. (FIG. 1A) A winner-take-all neural network, operating inside cells, can use a set of interacting proteins (Nⱼ) to activate exactly one of its outputs (colored proteases, yⱼ) depending on the relative values of its inputs (Xᵢ). (FIG. 1B) Formal description of the system in (FIG. 1A). The network can consist of m inputs (Xᵢ), each taking positive real values. The inputs can interact with the n nodes (Nⱼ) with weights wᵢⱼ (1≤i≤m, 1≤j≤n, 0≤wᵢⱼ≤1) connecting input Xᵢ to neuron Nⱼ. Each neuron can perform weighted sum operations to integrate the input signals it receives, and winner-take-all can be achieved through self-activation and mutual inhibition. The output yⱼ from a neuron Nⱼ is active only if its weighted sum is greater than that from any other neuron. (FIG. 1C) The decision boundary, α, for a 2-input, 2-neuron network can be tuned by varying the weights wᵢⱼ. (FIG. 1D) In a 2-input, 2-node circuit, each input protein activates either node protein by forming input-node complexes. Such complexes then undergo self-activation and mutual inhibition to perform the winner-take-all computation. The final state of the system is defined by the abundance of the active node. (FIG. 1E) The weighted sum operation can be carried out through competitive and cooperative binding. The two inputs are de novo designed orthogonal DHDs (X₁ and X₂). Each node can comprise of two groups of proteins: the N-nodes, where the cognate binding partners of X₁ and X₂ are caged by a genetically fused DHD caging domain, and further linked to the N-terminal half of a protease, its cleavage site, and a DHFR degron; the C-nodes, made from the cognate binding partner of the DHD caging domain in primary half-nodes, fused to the cleavage sequence of the other protease, and the C-terminal half of a protease. The inputs, N-node, and C-node can bind cooperatively, such that neither of the two proteins can bind with high affinity without the third protein. They also interact competitively, such that the N-nodes compete to bind to the input protein, and the C-nodes. Two types of intermediate products can result from these 3-way binding events: the active but destabilized proteases where the two protease halves reconstitute a functional protease, or the inactive and destabilized hybrids where the two protease halves do not match. The blue and green stripes indicate that the DHD domains can be either blue (X₁) or green (X₂). (FIG. 1F) Winner-take-all operation is achieved through two types of reactions: mutual inhibition, where each protease can inactivate the opposite protease type by cleaving its C-terminal half proteases off the C-nodes; self-activation, where the intermediate DHD-protease complexes cleave off DHFR degrons from their N-nodes, converting them to stable proteases, which can in turn activate and inhibit other protease complexes (additional complexes not shown). (FIG. 1G) The weights connecting inputs to neurons are set based on the abundance of each primary half-neuron complex. For example, w₁₁, the weight that connects input X₁ to Neuron 1, is defined as the concentration of the N₁₁ N-node divided by the sum of the concentrations of all N-nodes that can potentially bind to X₁, in this case N₁₁+N₁₂. (FIG. 1H) Legend for the circuit components depicted herein.

FIGS. 2A-2D depict non-limiting exemplary schematics and data related to simulated two-input circuits performing winner-take-all classification. (FIG. 2A) Simulations of circuit variants (left) reveal circuit dynamics (middle) and classification ability (right). Input values, weights, and weighted sums (denoted Σ) are indicated on the circuit diagrams, with larger weights represented by thicker lines. Each cell in the heatmap represents the difference between active N₁ and N₂ proteases at steady state. Both the full circuit and the comparator are able to classify across the full range of input levels, while circuits lacking self-activation or mutual inhibition only classify within a limited input range. (FIG. 2B) The decision boundary (gray lines) of the comparator circuit can be tuned by varying the relative levels of the two node proteins, N₁₁^{D} and N₂₂^{D}. (FIG. 2C) Stochastic simulations of the comparator. Twenty simulations were performed for each condition (light traces), and their average traces are plotted in dark lines. Colors indicate the input levels (legend). See supplementary materials for simulation methods. (FIG. 2D) Percentage of 50 equivalent simulations that correctly classify inputs as a function of the concentration difference between the two inputs. Input X₁ is fixed at 0.05 molecules/s. Even with stochasticity, input differences of at least 20% classify correctly ˜95% of the time.

FIGS. 3A-3G depict non-limiting exemplary schematics and data related to a winner-take-all neural network circuit classifying inputs in mammalian cells. (FIG. 3A) Plasmids and the encoded protein constructs. All plasmids use the human cytomegalovirus promoter (PCMV) to express each engineered protein. Deg denotes DHFR degron. Orange and yellow circles denote cleavage sites for corresponding proteases. DHDs are designed heterodimerizing proteins. A schematic representation of each fusion protein is shown to the right of the construct. (FIG. 3B) The stable reporter cell line constitutively co-expresses mCitrine and mCherry fluorescent proteins that can be cleaved at the N-terminus by TEVP and TVMVP, respectively, to reveal N-terminal degrons that destabilize the fluorescent proteins. PGK, 3-phosphoglycerate kinase promoter. (FIG. 3C) Engineered protease can respond to inputs, self-activate, and mutually inhibit. Normalized protease activities under different experimental setups indicate expected functions. (FIG. 3D) Testing the weight multiplication module by fixing input X₁ and varying nodes N₁₁^{D} and N₁₂^{D}. Ideal behaviors are shown in solid lines, experimental data points are mean±s.d from three biological repeats. (FIG. 3E) A fully connected 2-input, 2-neuron circuit that compares relative input levels (left). (FIG. 3F) A fully connected 2-input, 2-neuron circuit that, by construction, should always result in N₁ being the winner. (FIG. 3G) The decision boundaries of a two-input comparator can be tuned by varying the ratios of N₁ to N₂ protein concentrations. Data in FIGS. 3E-3G are averages of two biological replicates.

FIGS. 4A-4F depict non-limiting exemplary schematics and data related to scaling of the winner-take-all circuit. (FIG. 4A) An m-input, m-neuron comparator circuit. (FIG. 4B) Classification abilities of comparators that take 2, 3, and 4 inputs. As the size of the comparator increases, the ability to compare relative input levels is retained, while dynamic range is reduced due to the increased total number of substrates for each protease. (FIG. 4C) The number of reactions in a comparator circuit increases roughly linearly with its size, as the circuit dynamic range decreases. (FIG. 4D) A 2-input classifier can generate distinct responses to all 4 input states. (FIG. 4E) A 3-input winner-take-all circuit performs the (X₁ OR X₃) AND NOT X₂ calculation. Neuron 1 wins if the condition is met. (FIG. 4F) A 3-input winner-take-all circuit performs “Any 2 out of 3” logic. A fourth “hidden unit” input was added to set the threshold and make the circuit more compact. Neuron 1 wins if the condition is met.

FIGS. 5A-5F depict non-limiting exemplary schematics and data related to simulations of the winner-take-all neural network. (FIG. 5A) For a 2-input comparator, scanning the k_{cat} parameter values of the two proteases reveal that the circuit can correctly classify relative input levels as long as the protease corresponding to the larger input has a k_{cat} value of at least 0.04/s. (FIG. 5B) For a 2-input comparator, bigger differences between the basal degradation rate (y-axis) and degron-based degradation rates (x-axis) result in more accurate classification (colorbar). (FIG. 5C) Deterministic simulation of the 2-input comparator with varying input levels. The circuit can take longer to classify inputs that are increasingly similar to each other, and can distinguish inputs within 10% of each other under reasonable time. (FIG. 5D) A fully connected 2-input neural network generates similar simulated dynamics as the comparator in both deterministic (FIG. 5E) and stochastic (FIG. 5F) simulations.

FIG. 6 depicts non-limiting exemplary schematics of plasmids and the encoded protein constructs. PCMV, human cytomegalovirus promoter. PEF1α, human elongation factor-1 α promoter. PGK, 3-phosphoglycerate kinase promoter. Deg, DHFR degron. BFP, blue fluorescent protein. Schematics of the resulting constructs are shown on the right.

FIGS. 7A-7G depict non-limiting exemplary schematics and data related to experimental validation of the winner-take-all neural network. (FIG. 7A) To read out protease activities, the reporter cell line (FIG. 4B) expresses mCherry and mCitrine fluorescent proteins with N-end degrons that can be revealed upon protease cleavage by TVMVP and TEVP, respectively. Schematic shows how cleavage can expose degron (half-circle), to cause target protein degradation. (FIG. 7B) Flow cytometry histograms reveal that TVMVP and TEVP exclusively destabilize mCherry and mCitrine, respectively. (FIG. 7C) A representative flow cytometry histogram showing the activation of Node 2 by the N₂₂D protein and its input X₂. (FIG. 7D, FIG. 7E, and FIG. 7F) Activation of other nodes by their corresponding input proteins. (FIG. 7G) The node protein N₁₂D undergoes self-activation, and its activities can be inhibited by the opposing node protein N₁₁D.

FIG. 8 depicts proteins provided herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include synthetic protein circuits. The synthetic protein circuit can comprise: a first n-node protein comprising a first part of a first protease domain, a first dimerization domain, and a second dimerization domain. The synthetic protein circuit can comprise: a companion first n-node protein comprising a first part of a first protease domain, a third dimerization domain, and a fourth dimerization domain. The synthetic protein circuit can comprise: a first c-node protein comprising a second part of a first protease domain and a fifth dimerization domain. The synthetic protein circuit can comprise: a second n-node protein comprising a first part of a second protease domain, a sixth dimerization domain, and a seventh dimerization domain. The synthetic protein circuit can comprise: a companion second n-node protein comprising a first part of a second protease domain, an eighth dimerization domain, and a ninth dimerization domain. The synthetic protein circuit can comprise: a second c-node protein comprising a second part of a second protease domain and a tenth dimerization domain. The synthetic protein circuit can comprise: a first input protein comprising an eleventh dimerization domain. The synthetic protein circuit can comprise: a second input protein comprising a twelfth dimerization domain.

Disclosed herein include nucleic acid compositions. The nucleic acid composition can comprise: one or more polynucleotides encoding a synthetic protein circuit provided herein (or components thereof). Disclosed herein include systems for classifying multi-input signals in a cell, comprising one or more components of the synthetic protein circuits provided herein. Disclosed herein include systems for classifying the cell type and/or cell state of a cell, comprising one or more components of the synthetic protein circuits provided herein. Disclosed herein include engineered cells or a population of engineered cells, comprising: a synthetic protein circuit provided herein, one or more components of the synthetic protein circuits provided herein and/or the nucleic acid compositions provided herein. Disclosed herein include methods for classifying the cell type and/or cell state of a cell, comprising: expressing a synthetic protein circuit provided herein or one or more components of the synthetic protein circuits provided herein in the cell. Disclosed herein include methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

### A Synthetic Protein-Level Neural Network in Mammalian Cells

Artificial neural networks provide a powerful paradigm for information processing that has transformed diverse fields. Within living cells, genetically encoded synthetic molecular networks could, in principle, harness principles of neural computation to classify molecular signals. Herein de novo designed protein heterodimers and engineered viral proteases are combined to implement a synthetic protein circuit that performs winner-take-all neural network computation. This “perceptein” circuit includes modules that compute weighted sums of input protein concentrations through reversible binding interactions, and allow for self-activation and mutual inhibition of protein components using irreversible proteolytic cleavage reactions. Altogether, these interactions comprise a network of 310 chemical reactions stemming from 8 expressed protein species. The complete system achieves signal classification with tunable decision boundaries in mammalian cells (Example 1). The results provided herein demonstrate how engineered protein-based networks can enable programmable signal classification in living cells. There are provided, in some embodiments, synthetic protein circuits (termed Perceptein) based on the principle of winner-take-all neural computation that successfully classifies multi-input signals in mammalian cells. Synthetic protein-level neural networks are provided herein that can operate in living cells. Synthetic protein circuits are provided herein can operate within cells to perform computations analogous to those of artificial neural networks, including winner-take-all classification.

As shown herein, the key elements of winner take all neural computation can be implemented with modular protein domains (See Example 1). The perceptein architecture can combine de novo designed protein heterodimers and engineered split viral proteases. These proteins can combine into complexes (“nodes”) whose interactions perform weighted input summation, self-activation, and mutual inhibition to achieve winner-take-all dynamics, in which a single output protease is activated depending on the relative levels of the inputs. Thus, the perceptein design can act as a simple protein level neural network for classification.

Additionally, as shown herein, the perceptein successfully classifies protein level input signals in mammalian cells (See Example 1). Design, optimization, and analysis of engineered perceptein protein components shows that they function as predicted by mathematical models. Combining these components into the full circuit generates a predicted set of 310 chemical reactions involving 158 distinct proteins and protein complexes, stemming from just 8 starting proteins. Despite this complexity, the perceptein system successfully classified multi-input signals in mammalian cells, similar to predictions.

Furthermore, as shown herein, the circuits described herein allow tuning of decision boundaries in cells (See Example 1). Consistent with model predictions, the perceptein design allowed tuning of the decision boundary in living cells by varying the relative expression levels of different components without introducing additional species. Additional modeling shows how this system can further allow a range of other versatile computations and how it can scale to accommodate additional inputs. Together, the synthetic protein circuits provided herein and the results shown in Example 1 bring principles of neural computation to the level of designed proteins interacting within living cells.

The circuits described herein can be extended to allow for additional capabilities. For example, in order to process signals in cell therapeutic or other contexts, the perceptein input proteins can be activated by endogenous signaling pathways or synthetic signaling pathways such as the synNotch receptors. The outputs of the circuits described herein can include one or more of the following: the production or secretion of cytokines; the induction of cell death, including apoptosis, pyroptosis, and other forms of cell death; the initiation of cell movement, and differentiation into specific cell fates. In this way the circuits described herein can allow cells to specifically induce a wide variety of biological responses based on input classification. The circuits described herein can be extended analogously to recurrent neural networks, by allowing input proteins for one layer of the perceptein system to be activated by outputs from another or from the same layer. Additionally, the circuits described herein can incorporate principles of machine learning by incorporating methods such as back propagation to adjust the effective weights of different nodes. Furthermore, the circuits described herein can be scaled to incorporate more inputs and outputs by creating additional variants of the proteins shown herein, with orthogonal protease variants.

There are provided, in some embodiments, synthetic protein circuits. The synthetic protein circuit can comprise: a first n-node protein comprising a first part of a first protease domain, a first dimerization domain, and a second dimerization domain. The synthetic protein circuit can comprise: a companion first n-node protein comprising a first part of a first protease domain, a third dimerization domain, and a fourth dimerization domain. The synthetic protein circuit can comprise: a first c-node protein comprising a second part of a first protease domain and a fifth dimerization domain. The synthetic protein circuit can comprise: a second n-node protein comprising a first part of a second protease domain, a sixth dimerization domain, and a seventh dimerization domain. The synthetic protein circuit can comprise: a companion second n-node protein comprising a first part of a second protease domain, an eighth dimerization domain, and a ninth dimerization domain. The synthetic protein circuit can comprise: a second c-node protein comprising a second part of a second protease domain and a tenth dimerization domain. The synthetic protein circuit can comprise: a first input protein comprising an eleventh dimerization domain. The synthetic protein circuit can comprise: a second input protein comprising a twelfth dimerization domain.

In some embodiments, the first dimerization domain, the third dimerization domain, the sixth dimerization domain, and/or the eighth dimerization domain, are the same or have at least about 80% sequence identity. In some embodiments, the second dimerization domain and the seventh dimerization domain are the same or have at least about 80% sequence identity. In some embodiments, the fourth dimerization domain and the ninth dimerization domain are the same or have at least about 80% sequence identity. In some embodiments, the fifth dimerization domain and the tenth dimerization domain are the same or have at least about 80% sequence identity. In some embodiments, one or more the first dimerization domain, the third dimerization domain, the sixth dimerization domain, and the eighth dimerization domain are capable of binding one or more of the second dimerization domain, the seventh dimerization, the fourth dimerization domain, the ninth dimerization domain, the fifth dimerization domain, and the tenth dimerization domain. In some embodiments, the eleventh dimerization domain is capable of binding the second dimerization domain and/or the seventh dimerization domain. In some embodiments, the twelfth dimerization domain is capable of binding the fourth dimerization domain and/or the ninth dimerization domain.

In some embodiments, intramolecular binding between the first dimerization domain and the second dimerization domain of the first n-node protein is capable of preventing the first n-node protein from binding the first c-node protein to form a first complex. In some embodiments, intramolecular binding between the first dimerization domain and the second dimerization domain of the first n-node protein is capable of preventing the first n-node protein from binding the second c-node protein to form a second complex. In some embodiments, intramolecular binding between the third dimerization domain and the fourth dimerization domain of the companion first n-node protein is capable of preventing the companion first n-node protein from binding the first c-node protein to form a third complex. In some embodiments, intramolecular binding between the third dimerization domain and the fourth dimerization domain of the companion first n-node protein is capable of preventing the companion first n-node protein from binding the second c-node protein to form a fourth complex. In some embodiments, intramolecular binding between the sixth dimerization domain and the seventh dimerization domain of the second n-node protein is capable of preventing the second n-node protein from binding the first c-node protein to form a fifth complex. In some embodiments, intramolecular binding between the sixth dimerization domain and the seventh dimerization domain of the second n-node protein is capable of preventing the second n-node protein from binding the second c-node protein to form a sixth complex. In some embodiments, intramolecular binding between the eighth dimerization domain and the ninth dimerization domain of the companion second n-node protein is capable of preventing the companion second n-node protein from binding the first c-node protein to form a seventh complex. In some embodiments, intramolecular binding between the eighth dimerization domain and the ninth dimerization domain of the companion second n-node protein is capable of preventing the companion second n-node protein from binding the second c-node protein to form an eighth complex.

In some embodiments, the eleventh dimerization domain of the first input protein is capable of disrupting intramolecular binding between the first dimerization domain and the second dimerization domain of the first n-node protein. In some embodiments, the twelfth dimerization domain of the second input protein is capable of disrupting intramolecular binding between the third dimerization domain and the fourth dimerization domain of the companion first n-node protein. In some embodiments, the eleventh dimerization domain of the first input protein is capable of disrupting intramolecular binding between the sixth dimerization domain and the seventh dimerization domain of the second n-node protein. In some embodiments, the twelfth dimerization domain of the second input protein is capable of disrupting intramolecular binding between the eighth dimerization domain and the ninth dimerization domain of the companion second n-node protein.

In some embodiments, intermolecular binding between the eleventh dimerization domain of the first input protein and the second dimerization domain of the first n-node protein enables the first dimerization domain of the first n-node protein to bind the fifth dimerization domain of the first c-node protein and thereby form a first complex. In some embodiments, intermolecular binding between the eleventh dimerization domain of the first input protein and the second dimerization domain of the first n-node protein enables the first dimerization domain of the first n-node protein to bind the tenth dimerization domain of the second c-node protein and thereby form a second complex. In some embodiments, intermolecular binding between the twelfth dimerization domain of the second input protein and the fourth dimerization domain of the companion first n-node protein enables the third dimerization domain of the companion first n-node protein to bind the fifth dimerization domain of the first c-node protein and thereby form a third complex. In some embodiments, intermolecular binding between the twelfth dimerization domain of the second input protein and the fourth dimerization domain of the companion first n-node protein enables the third dimerization domain of the companion first n-node protein to bind the tenth dimerization domain of the second c-node protein and thereby form a fourth complex. In some embodiments, intermolecular binding between the eleventh dimerization domain of the first input protein and the seventh dimerization domain of the second n-node protein enables sixth dimerization domain of the second n-node protein to bind the fifth dimerization domain of the first c-node protein and thereby form a fifth complex. In some embodiments, intermolecular binding between the eleventh dimerization domain of the first input protein and the seventh dimerization domain of the second n-node protein enables sixth dimerization domain of the second n-node protein to bind the tenth dimerization domain of the second c-node protein and thereby form a sixth complex. In some embodiments, intermolecular binding between the twelfth dimerization domain of the second input protein and the ninth dimerization domain of the companion second n-node protein enables the eighth dimerization domain of the companion second n-node protein to bind the fifth dimerization domain of the first c-node protein and thereby form a seventh complex. In some embodiments, intermolecular binding between the twelfth dimerization domain of the second input protein and the ninth dimerization domain of the companion second n-node protein enables the eighth dimerization domain of the companion second n-node protein to bind the tenth dimerization domain of the second c-node protein and thereby form an eighth complex.

In some embodiments, the first complex and/or the third complex comprise a first protease capable of being in a first protease active state. In some embodiments, the sixth complex and/or the eighth complex comprise a second protease capable of being in a second protease active state. In some embodiments, the second complex, fourth complex, fifth complex, and/or seventh complex do not comprise a first protease capable of being in a first protease active state or a second protease capable of being in a second protease active state. In some embodiments, the first part of the first protease domain and the second part of the first protease domain are capable of associating with each other to constitute a first protease capable of being in a first protease active state when: (i) the first n-node protein binds the first c-node protein to form a first complex; and/or (ii) the companion first n-node protein binds the first c-node protein to form a third complex. In some embodiments, the first part of the second protease domain and the second part of the second protease domain are capable of associating with each other to constitute a second protease capable of being in a second protease active state when: (i) the second n-node protein binds the second c-node protein to form a sixth complex; and/or (ii) the companion second n-node protein binds the second c-node protein to form an eighth complex. In some embodiments, the first n-node protein, the companion first n-node protein, the second n-node protein, and/or the companion second n-node protein comprise a degradation domain, optionally the presence of said degradation domain causes the protein to be a destabilized state.

In some embodiments, the first n-node protein and/or the companion first n-node protein comprise a first cut site the first protease in the first protease active state is capable of cutting, optionally the cleavage of said first cut site is capable of inactivating or removing the degradation domain, thereby causing the protein to be in stabilized state. In some embodiments, the second n-node protein and/or the companion second n-node protein comprise a second cut site the second protease in the second protease active state is capable of cutting, optionally the cleavage of said second cut site is capable of inactivating or removing the degradation domain, thereby causing the protein to be in stabilized state. In some embodiments, the first c-node protein comprises a second cut site the second protease in the second protease active state is capable of cutting, optionally the second cut site is situated between the second part of a first protease domain and the fifth dimerization domain, further optionally cleavage of the said second cut site prevents said first c-node protein from constituting a complex with active protease activity. In some embodiments, the second c-node protein comprises a first cut site the first protease in the first protease active state is capable of cutting, optionally the first cut site is situated between the second part of a second protease domain and the tenth dimerization domain, further optionally cleavage of the said first cut site prevents said second c-node protein from constituting a complex with active protease activity.

First complexes and/or third complexes can be capable of self-activation via first protease-mediated cleavage of the first cut site of first complexes and/or third complexes. In some embodiments, sixth complexes and/or eighth complexes are capable of self-activation via second protease-mediated cleavage of the second cut site of sixth complexes and/or eighth complexes. In some embodiments, first complexes and/or third complexes are capable of mutual inhibition via first protease-mediated cleavage of the first cut site of sixth complexes and/or eighth complexes. In some embodiments, sixth complexes and/or eighth complexes are capable of mutual inhibition via second protease-mediated cleavage of the first cut site of first complexes and/or third complexes.

The degradation domain can comprise a degron, optionally the degron comprises an N-degron, a dihydrofolate reductase (DHFR) degron, a FKB protein (FKBP) degron, derivatives thereof, or any combination thereof. In some embodiments, the first protease and/or the second protease comprises a viral protease. In some embodiments, the first protease and/or the second protease comprises tobacco etch virus (TEV) protease, tobacco vein mottling virus (TVMV) protease, hepatitis C virus protease (HCVP), derivatives thereof, or any combination thereof. In some embodiments, the first n-node protein, the companion first n-node protein, the first c-node protein, the second n-node protein, the companion second n-node protein, and/or the second c-node protein comprises a linker, optionally the linker is: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more.

The systems, methods, compositions, and kits provided herein can, in some embodiments, be employed in concert with the designed heterodimer proteins, monomeric polypeptides capable of forming heterodimer proteins described in PCT Patent Publication No. WO2020093043, entitled, “Orthogonal protein heterodimers,” the content of which is incorporated herein by reference in its entirety. The systems, methods, compositions, and kits provided herein can, in some embodiments, be employed in concert with elements described in Chen et al. (Nature, 2019), Thomas et al. (Journal of the American Chemical Society, 2013), Gradišar and Jerala (Journal of Peptide Science, 2011), Reinke et al (Journal of the American Chemical Society, 2010), and Prehoda et al. (Science, 2000), the contents of which are incorporated herein by reference in their entirety. A pair of dimerization domains capable of binding each other can include a DHD heterodimer a polypeptide and a DHD heterodimer b polypeptide. One or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain can be selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

One or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain can comprise or can be derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, one or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain is a homodimerization domain or a multimerization domain, optionally a homo- or hetero-dimerizing or multimerizing leucine zipper, a PDZ domains, a SH3 domain, aGBD domain, or any combination thereof.

In some embodiments, the first n-node protein, the companion first n-node protein, and/or the first c-node protein constitute a first node of the synthetic protein circuit having first protease activity, and wherein the second n-node protein, the companion second n-node protein, and/or the second c-node protein constitute a second node of the synthetic protein circuit having second protease activity, optionally the synthetic protein circuit activates a single node. In some embodiments, the synthetic protein circuit further comprises m supplemental input proteins and/or n supplemental nodes, wherein n or m is an integer greater than zero.

In some embodiments, the synthetic protein circuit does not comprise the companion first n-node protein and/or the second n-node protein. In some embodiments, the synthetic protein circuit achieves winner-take-all dynamics with tunable decision boundaries, wherein winner-take-all dynamics comprises a single protease activated in the synthetic protein circuit. In some embodiments, the output of the synthetic protein circuit depends on (i) the relative levels of the first input protein and the second input protein and (ii) the tunable decision boundaries of the synthetic protein circuit. In some embodiments, the output of the synthetic protein circuit is either the first protease in the first protease active state or the second protease in the second protease active state. In some embodiments, the first protease in the first protease active state and/or the second protease in the second protease active state is capable of modulating the expression, localization, and/or stability of one or more input proteins of a downstream synthetic protein circuit. In some embodiments, the relative levels of the first input protein and/or the second input protein is modulated by an upstream synthetic protein circuit.

The relative levels of the first input protein and/or the second input protein can be capable of being regulated via one or more of expression, localization, and stability. In some embodiments, the synthetic protein circuit is capable of winner-take-all classification of biological information, wherein said biological information is associated with the relative levels of the first input protein and the second input protein, further optionally said biological information relates to the presence and/or amount of a unique cell type and/or a unique cell state in a cell. In some embodiments, the relative levels of the first input protein and the second input protein is correlated with the presence and/or amount of a unique cell type and/or a unique cell state in a cell. In some embodiments, the degree of expression and/or degradation of the first input protein and/or the second input protein is associated with the presence and/or amount of a unique cell type and/or a unique cell state. In some embodiments, an input protein comprises a degradation domain and a cut site, wherein a protease is capable of cutting the cut site of the input protein to hide the degradation domain, and wherein the degradation domain of the input protein being hidden changes the input protein to an input protein stabilized state. In some embodiments, the input protein comprises a degradation domain and a cut site, wherein a protease is capable of cutting the cut site of the input protein to expose the degradation domain, and wherein the degradation domain of the input protein being exposed changes the input protein to an input protein destabilized state. The synthetic protein circuit can comprise: one or more modulator circuit proteins configured to regulate the expression and/or stability of the first input protein and/or the second input protein in response to the cell type and/or cell state of a cell.

The tunable decision boundaries can be capable of being tuned by adjusting the relative levels of the first n-node protein, the companion first n-node protein, the first c-node protein, the second n-node protein, the companion second n-node protein, and/or the second c-node protein, optionally said adjusting comprises differential expression. In some embodiments, the tunable decision boundaries are capable of being tuned by introducing one or more amino acid substitutions into the degradation domains(s), the cut site(s) and/or the dimerization domains(s).

The synthetic protein circuit can be present in a cell. In some embodiments, the cell is: a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, or an in situ cell. In some embodiments, the cell is a eukaryotic cell, optionally a mammalian cell, further optionally the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

The synthetic protein circuit can be capable of modulating the expression, concentration, localization, stability, and/or activity of the one or more exogenous payload protein(s) and/or one or more endogenous payload protein(s) of a cell. In some embodiments, the first protease in the first protease active state or the second protease in the second protease active state is capable of modulating the expression, concentration, localization, stability, and/or activity of the one or more exogenous payload protein(s) and/or one or more endogenous payload protein(s) of a cell In some embodiments, the first protease in the first protease active state or the second protease in the second protease active state is capable of modulating the cell type and/or cell state of a cell based on the presence and/or amount of a unique cell type and/or a unique cell state in a cell. In some embodiments, the synthetic protein circuit further comprises one or more effector proteins comprising a first cut site the first protease in the first protease active state is capable of cutting or a second cut site the second protease in the second protease active state is capable of cutting, thereby modulating its expression, concentration, localization, stability, and/or activity, optionally said one or more effector proteins are capable of modulating the expression, concentration, localization, stability, and/or activity of the one or more exogenous payload protein(s) and/or one or more endogenous payload protein(s) of a cell. The synthetic protein circuits provided herein can further comprise one or more payloads (e.g., one or more exogenous payload proteins).

The synthetic protein circuit can be configured to be responsive to changes in: cell environment, optionally cell environment comprises location relative to a target site of a subject and/or changes in the presence and/or absence of cell(s) of interest, optionally said cell(s) of interest comprise target-specific antigen(s); one or more signal transduction pathways regulating cell survival, cell growth, cell proliferation, cell adhesion, cell migration, cell metabolism, cell morphology, cell differentiation, apoptosis, or any combination thereof input(s) of a synthetic receptor system, optionally Synthetic Notch (SynNotch) receptor, a Modular Extracellular Sensor Architecture (MESA) receptor, a synthekine, Tango, dCas9-synR, a chimeric antigen receptor, or any combination thereof and/or T cell activity, optionally T cell activity comprises one or more of T cell simulation, T cell activation, cytokine secretion, T cell survival, T cell proliferation, CTL activity, T cell degranulation, and T cell differentiation.

In some embodiments, the synthetic protein circuit is: (i) capable of modulating cell states, cell types, and/or cell behaviors, (ii) configured to selectively activate cell death and/or immune recruitment to tumor cells; and/or (iii) is configured to detect the intracellular state of a cell and classify it as tumor or normal based on the levels or activities of relevant molecules or pathways. In some embodiments, a unique cell type and/or a unique cell state comprises a unique gene expression pattern, optionally the unique cell type and/or unique cell state comprises a unique anatomic location, further optionally the unique cell type and/or the unique cell state comprises anatomically locally unique gene expression. In some embodiments, a unique cell type and/or a unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment, optionally the senescent cell state induced by a tumor microenvironment comprises expression of CD57, KRLG1, TIGIT, or any combination thereof. In some embodiments, the unique cell state and/or unique cell type is characterized by aberrant signaling of one or more signal transducer(s). In some embodiments, the unique cell state comprises: a physiological state, optionally a cell cycle state, a differentiation state, a development state a metabolic state, or a combination thereof; and/or a pathological state, optionally a disease state, a human disease state, a diabetic state, an immune disorder state, a neurodegenerative disorder state, an oncogenic state, or a combination thereof. In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion. In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety. The systems, methods, compositions, and kits provided herein can, in some embodiments, be employed in concert with the systems, methods, compositions, and kits for described in U.S. Patent Publication No. 2023/0076395, entitled, “CELL-TO-CELL DELIVERY OF RNA CIRCUITS,” and in U.S. Patent Publication No. 2023/0071834, entitled, “EXPORTED RNA REPORTERS FOR LIVE-CELL MEASUREMENT,” the contents of which are incorporated herein by reference in their entirety. The systems, methods, compositions, and kits provided herein can, in some embodiments, be employed in concert with the systems, methods, compositions, and kits for described in Chen, Zibo, et al. (“A synthetic protein-level neural network in mammalian cells.” bioRxiv (2022): 2022-07) the content of which is incorporated herein by reference in its entirety.

There are provided, in some embodiments, nucleic acid compositions. The nucleic acid composition can comprise: one or more polynucleotides encoding a synthetic protein circuit provided herein (or components thereof). One or more components of the synthetic protein systems provided herein can comprise an amino acid sequence that is at least 80%, 85%, 90%, 95%, 98%, 99%, or 100% identical to any one of SEQ ID NOS: 1-25, or portions thereof. In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the one or more polynucleotides are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof. In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m1Ψ), and 5-methyl-uridine (m5U).

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors, optionally at least one of the one or more vectors is a viral vector, a plasmid, a transposable element, a naked DNA vector, a lipid nanoparticle (LNP), or any combination thereof, optionally the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and optionally the transposable element is piggybac transposon or sleeping beauty transposon. In some embodiments, the one or more polynucleotides are comprised in the one or more vectors, optionally the one or more polynucleotides are comprised in the same vector and/or different vectors, optionally the one or more polynucleotides are situated on the same nucleic acid and/or different nucleic acids. In some embodiments, the nucleic acid composition is configured to achieve relative levels of the first input protein and/or second input protein dependent on a unique cell type and/or unique cell state. In some embodiments, the nucleic acid composition is configured to achieve relative levels of the first n-node protein, the companion first n-node protein, the first c-node protein, the second n-node protein, the companion second n-node protein, and/or the second c-node protein desired by a user. Disclosed herein include compositions (e.g., pharmaceutical compositions) comprising a nucleic acid composition provided herein.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

There are provided, in some embodiments, systems for classifying multi-input signals in a cell, comprising one or more components of the synthetic protein circuits provided herein. Disclosed herein include systems for classifying the cell type and/or cell state of a cell, comprising one or more components of the synthetic protein circuits provided herein. There are provided, in some embodiments, engineered cell(s) or a population of engineered cells, comprising: a synthetic protein circuit provided herein, one or more components of the synthetic protein circuits provided herein and/or the nucleic acid compositions provided herein. There are provided, in some embodiments, methods for classifying the cell type and/or cell state of a cell, comprising: expressing a synthetic protein circuit provided herein or one or more components of the synthetic protein circuits provided herein in the cell.

### Payload Proteins

A payload protein can comprise an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3). The one or more payloads can comprise a secretion tag. The secretion tag can be selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. A payload protein can comprise a constitutive signal peptide for protein degradation (e.g., PEST). A payload protein can comprise a nuclear localization signal (NLS) or a nuclear export signal (NES). A payload protein can comprise a dosage indicator protein. The dosage indicator protein can be detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

The payload protein can comprise a synthetic protein circuit component. In some embodiments, the payload comprises a bispecific T cell engager (BiTE). In some embodiments, the orthogonal signal triggers cellular differentiation. The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof. The payload protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the payload protein can diminish immune cell function. The payload protein can be an activity regulator. The activity regulator can be capable of reducing T cell activity. The activity regulator can comprise a ubiquitin ligase involved in TCR/CAR signal transduction selected from the group comprising c-CBL, CBL-B, ITCH, R F125, R F128, WWP2, or any combination thereof. The activity regulator can comprise a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. The activity regulator can be a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. The activity regulator can be a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAP1, CRKII, C3G, WAVE2, ARP2/3, ABL, ADAP, RIAM, SKAP55, or any combination thereof. The activity regulator can comprise the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. The activity regulator can be targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

The payload protein can comprise a cytokine. The cytokine can be selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

The payload protein can comprise a member of the TGF-β/BMP family selected from the group consisting of TGF-β1, TGF-β2, TGF-β3, BMP-2, BMP-3a, BMP-3b, BMP-4, BMP-BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-GDF-6, GDF-7, GDF-8, GDF-9, GDF-12, GDF-14, mullerian inhibiting substance (MIS), activin-1, activin-2, activin-3, activin-4, and activin-5. The payload protein can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein can comprise a member of the immunoglobulin superfamily of cytokines selected from the group consisting of B7.1 (CD80) and B7.2 (B70). The payload protein can comprise an interferon. The interferon can be selected from interferon alpha, interferon beta, or interferon gamma. The payload protein can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein can comprise a interleukin. The interleukin can be selected from IL-10 IL-12, IL-1, IL-6, IL-7, IL-15, IL-2, IL-18 or IL-21. The payload protein can comprise a tumor necrosis factor (TNF). The TNF can be selected from TNF-alpha, TNF-beta, TNF-gamma, CD252, CD154, CD178, CD70, CD153, or 4-1BBL.

The payload protein can comprise a programmable nuclease. In some embodiments, the synthetic protein circuit senses correction of an aberrant locus by said programmable nuclease and reduces effector protein localization and/or activity. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the synthetic protein circuit comprises a polynucleotide encoding (i) a targeting molecule and/or (ii) a donor nucleic acid. In some embodiments, the targeting molecule is capable of associating with the programmable nuclease. In some embodiments, wherein the targeting molecule comprises single strand DNA or single strand RNA. In some embodiments, the targeting molecule comprises a single guide RNA (sgRNA).

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload protein is a pro-survival protein (e.g., Bcl-2, Bcl-XL, Mcl-1 and A1). In some embodiments, the payload protein comprises a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrome C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload protein is a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof. In some embodiments, the payload protein is a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload protein is a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein expression in the cell can vary. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

In some embodiments, the payload protein comprises a prodrug-converting enzyme. In some embodiments, the payload protein comprises a pro-death protein capable of halting cell growth and/or inducing cell death. The pro-death protein can be capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, the pro-death protein is capable of halting cell growth and/or inducing cell death in the presence of a pro-death agent. Any suitable pro-death protein and pro-death agent (e.g., prodrug) is contemplated this disclosure, such as, for example, the suicide gene/prodrug combinations depicted in Table 1. Methods provided herein can comprise administering a prodrug and/or pro-death agent.

TABLE 1
PRO-DEATH PROTEINS AND PRODRUGS

| Pro-death Proteins | Prodrug(s) |
|---|---|
| HSV thymidine kinase (TK) | Ganciclovir (GCV); Ganciclovir elaidic acid ester; |
| | Penciclovir (PCV); Acyclovir (ACV); Valacyclovir (VCV); |
| | (E)-5-(2-bromovinyl)-2′-deoxyuridine (BVDU); Zidovuline |
| | (AZT); 2′-exo-methanocarbathymidine (MCT) |
| Cytosine Deaminase (CD) | 5-fluorocytosine (5-FC) |
| Purine nucleoside | 6-methylpurine deoxyriboside (MEP); fludarabine (FAMP) |
| phosphorylase (PNP) | |
| Cytochrome p450 enzymes | Cyclophosphamide (CPA); Ifosfamide (IFO); 4-ipomeanol |
| (CYP) | (4-IM) |
| Carboxypeptidases (CP) | 4-[(2-chloroethyl)(2-mesyloxyethyl)amino]benzoyl-L- |
| | glutamic acid (CMDA); Hydroxy-and amino-aniline |
| | mustards; Anthracycline glutamates; Methotrexate α- |
| | peptides (MTX-Phe) |
| Caspase-9 | AP1903 |
| Carboxylesterase (CE) | Irinotecan (IRT); Anthracycline acetals |
| Nitroreductase (NTR) | dinitroaziridinylbenzamide CB1954; dinitrobenzamide |
| | mustard SN23862; 4-Nitrobenzyl carbamates; Quinones |
| Horse radish peroxidase | Indole-3-acetic acid (IAA); 5-Fluoroindole-3-acetic acid |
| (HRP) | (FIAA) |
| Guanine Ribosyltransferase | 6-Thioxanthine (6-TX) |
| (XGRTP) | |
| Glycosidase enzymes | HM1826; Anthracycline acetals |
| Methionine-α,γ-lyase (MET) | Selenomethionine (SeMET) |
| Thymidine phosphorylase (TP) | 5′-Deoxy-5-fluorouridine (5′-DFU) |

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both.

Payloads Modulating Signaling Pathways

A cell can be characterized by aberrant signaling of one or more signal transducers. In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. The aberrant signaling can comprise an aberrant signal of at least one signal transduction pathway regulating cell survival, cell growth, cell proliferation, cell adhesion, cell migration, cell metabolism, cell morphology, cell differentiation, apoptosis, or any combination thereof. The disease or disorder can be characterized by an aberrant signaling of the first transducer. The synthetic protein circuits provided herein can be capable of detecting aberrant signaling, an activity of a signal transducer, an activity of a signal transducer activator and/or an activity of a signal transducer repressor. The synthetic protein circuits provided herein can be capable of directly or indirectly inducing cell death in the presence of the aberrant signaling of one or more signal transducer(s). The synthetic protein circuits provided herein can be capable of modulating the degree of signaling in one or more signaling pathways, thereby treating or preventing a disease or disorder. Examples of payload proteins include those associated with a signaling biochemical pathway, e.g., a signaling biochemical pathway-associated gene or polynucleotide (e.g., a signal transducer). Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 2-4. In some embodiments, the methods and compositions disclosed herein prevent or treat one or more of the diseases and disorders listed in Tables 2-4. In some embodiments, the payload(s) and/or synthetic protein circuits provided herein comprises a replacement version of the signal transducer. In some embodiments, the methods and compositions further comprise knockdown of the corresponding endogenous signal transducer. The payload(s) and/or synthetic protein circuits provided herein can comprise the product of a gene listed in listed in Tables 2-4. In some embodiments, the payload(s) and/or synthetic protein circuits provided herein ameliorates a disease or disorder characterized by an aberrant signaling of one or more signaling transducers. In some embodiments, the payload(s) and/or synthetic protein circuits provided herein diminishes the activation level of one or more signal transducers (e.g., signal transducers with aberrant overactive signaling, signal transducers listed in Tables 2-4). In some embodiments, the payload(s) and/or synthetic protein circuits provided herein increases the activation level of one or more signal transducers (e.g., signal transducers with aberrant underactive signaling). In some such embodiments, the payload(s) and/or synthetic protein circuits provided herein can modulate the abundance, location, stability, and/or activity of activators or repressors of said signal transducers.

TABLE 2
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; Notch2; Notch3; |
| | Notch4; AKT; AKT2; AKT3; HIF; HIFla; HIF3a; Met; HRG; Bcl2; PPAR alpha; |
| | PPAR gamma; WT1 (Wilms Tumor); FGF Receptor Family members (5 |
| | members: 1, 2, 3, 4, 5); CDKN2a; APC; RB (retinoblastoma); MEN1; VHL; |
| | BRCA1; BRCA2; AR (Androgen Receptor); TSG101; IGF; IGF Receptor; Igf1 (4 |
| | variants); Igf2 (3 variants); Igf 1 Receptor; Igf 2 Receptor; Bax; Bcl2; caspases |
| | family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 (Cplx1); Tph1 |
| | Tryptophan hydroxylase; Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; DAOA; DTNBP1; Dao (Dao1) |
| Trinucleotide Repeat | HTT (Huntington's Dx); SBMA/SMAX1/AR (Kennedy's Dx); FXN/X25 |
| Disorders | (Friedrich's Ataxia); ATX3 (Machado- Joseph's Dx); ATXN1 and ATXN2 |
| | (spinocerebellar ataxias); DMPK (myotonic dystrophy); Atrophin-1 and Atn1 |
| | (DRPLA Dx); CBP (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related disorders | Prp |
| ALS | SOD1; ALS2; STEX; FUS; TARDBP; VEGF (VEGF-a; VEGF-b; VEGF-c) |
| Drug addiction | Prkce (alcohol); Drd2; Drd4; ABAT (alcohol); GRIA2; Grm5; Grin1; Htr1b; |
| | Grin2a; Drd3; Pdyn; Gria1 (alcohol) |
| Autism | Mecp2; BZRAP1; MDGA2; Sema5A; Neurexin 1; Fragile X (FMR2 (AFF2); |
| | FXR1; FXR2; Mglur5) |
| Alzheimer's Disease | E1; CHIP; UCH; UBB; Tau; LRP; PICALM; Clusterin; PS1; SORL1; CR1; |
| | Vldlr; Uba1; Uba3; CHIP28 (Aqp1, Aquaporin 1); Uchl1; Uchl3; APP |
| Inflammation | IL-10; IL-1 (IL-1a; IL-1b); IL-13; IL-17 (IL-17a (CTLA8); IL- 17b; IL-17c; IL- |
| | 17d; IL-17f); II-23; Cx3cr1; ptpn22; TNFa; NOD2/CARD15 for IBD; IL-6; IL-12 |
| | (IL-12a; IL-12b); CTLA4; Cx3cl1 |
| Parkinson's Disease | x-Synuclein; DJ-1; LRRK2; Parkin; PINK1 |

TABLE 3
SIGNAL TRANSDUCERS

| | |
|---|---|
| Blood and | Anemia (CDAN1, CDA1, RPS19, DBA, PKLR, PK1, NT5C3, UMPH1, PSN1, RHAG, |
| coagulation | RH50A, NRAMP2, SPTB, ALAS2, ANH1, ASB, ABCB7, ABC7, ASAT); Bare |
| diseases and | lymphocyte syndrome (TAPBP, TPSN, TAP2, ABCB3, PSF2, RING11, MHC2TA, |
| disorders | C2TA, RFX5, RFXAP, RFX5); Bleeding disorders (TBXA2R, P2RX1, P2X1); Factor |
| | H and factor H-like 1 (HF1, CFH, HUS); Factor V and factor VIII (MCFD2); Factor |
| | VII deficiency (F7); Factor X deficiency (F10); Factor XI deficiency (F11); Factor XII |
| | deficiency (F12, HAF); Factor XIIIA deficiency (F13A1, F13A); Factor XIIIB |
| | deficiency (F13B); Fanconi anemia (FANCA, FACA, FA1, FA, FAA, FAAP95, |
| | FAAP90, FLJ34064, FANCB, FANCC, FACC, BRCA2, FANCD1, FANCD2, FANCD, |
| | FACD, FAD, FANCE, FACE, FANCF, XRCC9, FANCG, BRIP1, BACH1, FANCJ, |
| | PHF9, FANCL, FANCM, KIAA1596); Hemophagocytic lymphohistiocytosis disorders |
| | (PRF1, HPLH2, UNC13D, MUNC13-4, HPLH3, HLH3, FHL3); Hemophilia A (F8, |
| | F8C, HEMA); Hemophilia B (F9, HEMB), Hemorrhagic disorders (PI, ATT, F5); |
| | Leukocyde deficiencies and disorders (ITGB2, CD18, LCAMB, LAD, EIF2B1, |
| | EIF2BA, EIF2B2, EIF2B3, EIF2B5, LVWM, CACH, CLE, EIF2B4); Sickle cell anemia |
| | (HBB); Thalassemia (HBA2, HBB, HBD, LCRB, HBA1). |
| Cell | B-cell non-Hodgkin lymphoma (BCL7A, BCL7); Leukemia (TAL1 TCL5, SCL, TAL2, |
| dysregulation | FLT3, NBS1, NBS, ZNFN1A1, IK1, LYF1, HOXD4, HOX4B, BCR, CML, PHL, ALL, |
| and oncology | ARNT, KRAS2, RASK2, GMPS, AF10, ARHGEF12, LARG, KIAA0382, CALM, |
| diseases and | CLTH, CEBPA, CEBP, CHIC2, BTL, FLT3, KIT, PBT, LPP, NPM1, NUP214, D9S46E, |
| disorders | CAN, CAIN, RUNX1, CBFA2, AML1, WHSC1L1, NSD3, FLT3, AF1Q, NPM1, |
| | NUMA1, ZNF145, PLZF, PML, MYL, STAT5B, AF10, CALM, CLTH, ARL11, |
| | ARLTS1, P2RX7, P2X7, BCR, CML, PHL, ALL, GRAF, NF1, VRNF, WSS, NFNS, |
| | PTPN11, PTP2C, SHP2, NS1, BCL2, CCND1, PRAD1, BCL1, TCRA, GATA1, GF1, |
| | ERYF1, NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, SDF1); |
| and immune | Autoimmune lymphoproliferative syndrome (TNFRSF6, APT1, FAS, CD95, |
| related | ALPS1A); Combined immunodeficiency, (IL2RG, SCIDX1, SCIDX, IMD4); HIV-1 |
| diseases and | (CCL5, SCYA5, D17S136E, TCP228), HIV susceptibility or infection (IL10, CSIF, |
| disorders | CMKBR2, CCR2, CMKBR5, CCCKR5 (CCR5)); Immunodeficiencies (CD3E, CD3G, |
| | AICDA, AID, HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); Inflammation |
| | (IL-10, IL-1 (IL-1a, IL-1b), IL-13, IL-17 (IL-17a (CTLA8), IL-17b, IL-17c, IL-17d, IL- |
| | 17f), II-23, Cx3cr1, ptpn22, TNFa, NOD2/CARD15 for IBD, IL-6, IL-12 (IL-12a, IL- |
| | 12b), CTLA4, Cx3cl1); Severe combined immunodeficiencies (SCIDs)(JAK3, JAKL, |
| | DCLRE1C, ARTEMIS, SCIDA, RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis (APOA1, APP, AAA, CVAP, AD1, |
| liver, kidney | GSN, FGA, LYZ, TTR, PALB); Cirrhosis (KRT18, KRT8, CIRH1A, NAIC, TEX292, |
| and protein | KIAA1988); Cystic fibrosis (CFTR, ABCC7, CF, MRP7); Glycogen storage diseases |
| diseases and | (SLC2A2, GLUT2, G6PC, G6PT, G6PT1, GAA, LAMP2, LAMPB, AGL, GDE, GBE1, |
| disorders | GYS2, PYGL, PFKM); Hepatic adenoma (TCF1, HNF1A, MODY3), Hepatic failure, |
| | early onset, and neurologic disorder (SCOD1, SCO1), Hepatic lipase deficiency |
| | (LIPC), Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, CASP8, |
| | MCH5); Medullary cystic kidney disease (UMOD, HNFJ, FJHN, MCKD2, |
| | ADMCKD2); Phenylketonuria (PAH, PKU1, QDPR, DHPR, PTS); Polycystic kidney |
| | and hepatic disease (FCYT, PKHD1, ARPKD, PKD1, PKD2, PKD4, PKDTS, PRKCSH, |
| | G19P1, PCLD, SEC63). |
| Muscular/ | Becker muscular dystrophy (DMD, BMD, MYF6), Duchenne Muscular Dystrophy |
| Skeletal | (DMD, BMD); Emery-Dreifuss muscular dystrophy (LMNA, LMN1, EMD2, FPLD, |
| diseases and | CMD1A, HGPS, LGMD1B, LMNA, LMN1, EMD2, FPLD, CMD1A); |
| disorders | Facioscapulohumeral muscular dystrophy (FSHMD1A, FSHD1A); Muscular |
| | dystrophy (FKRP, MDC1C, LGMD2I, LAMA2, LAMM, LARGE, KIAA0609, MDC1D, |
| | FCMD, TTID, MYOT, CAPN3, CANP3, DYSF, LGMD2B, SGCG, LGMD2C, DMDA1, |
| | SCG3, SGCA, ADL, DAG2, LGMD2D, DMDA2, SGCB, LGMD2E, SGCD, SGD, |
| | LGMD2F, CMD1L, TCAP, LGMD2G, CMD1N, TRIM32, HT2A, LGMD2H, FKRP, |
| | MDC1C, LGMD2I, TTN, CMD1G, TMD, LGMD2J, POMT1, CAV3, LGMD1C, |
| | SEPN1, SELN, RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, TIRC7, OC116, |
| | OPTB1); Muscular atrophy (VAPB, VAPC, ALS8, SMN1, SMA1, SMA2, SMA3, |
| | SMA4, BSCL2, SPG17, GARS, SMAD1, CMT2D, HEXB, IGHMBP2, SMUBP2, |
| | CATF1, SMARD1). |
| Neurological | ALS (SOD1, ALS2, STEX, FUS, TARDBP, VEGF (VEGF-a, VEGF-b, VEGF-c); |
| and neuronal | Alzheimer disease (APP, AAA, CVAP, AD1, APOE, AD2, PSEN2, AD4, STM2, |
| diseases and | APBB2, FE65L1, NOS3, PLAU, URK, ACE, DCP1, ACE1, MPO, PACIP1, PAXIP1L, |
| disorders | PTIP, A2M, BLMH, BMH, PSEN1, AD3); Autism (Mecp2, BZRAP1, MDGA2, |
| | Sema5A, Neurexin 1, GLO1, MECP2, RTT, PPMX, MRX16, MRX79, NLGN3, NLGN4, |
| | KIAA1260, AUTSX2); Fragile X Syndrome (FMR2, FXR1, FXR2, mGLUR5); |
| | Huntington's disease and disease like disorders (HD, IT15, PRNP, PRIP, JPH3, JP3, |
| | HDL2, TBP, SCA17); Parkinson disease (NR4A2, NURR1, NOT, TINUR, SNCAIP, |
| | TBP, SCA17, SNCA, NACP, PARK1, PARK4, DJ1, PARK7, LRRK2, PARK8, PINK1, |
| | PARK6, UCHL1, PARK5, SNCA, NACP, PARK1, PARK4, PRKN, PARK2, PDJ, DBH, |
| | NDUFV2); Rett syndrome (MECP2, RTT, PPMX, MRX16, MRX79, CDKL5, STK9, |
| | MECP2, RTT, PPMX, MRX16, MRX79, x-Synuclein, DJ-1); Schizophrenia |
| | (Neuregulin1 (Nrg1), Erb4 (receptor for Neuregulin), Complexin1 (Cplx1), Tph1 |
| | Tryptophan hydroxylase, Tph2, Tryptophan hydroxylase 2, Neurexin 1, GSK3, GSK3a, |
| | GSK3b, 5-HTT (Slc6a4), COMT, DRD (Drd1a), SLC6A3, DAOA, DTNBP1, Dao |
| | (Dao1)); Secretase Related Disorders (APH-1 (alpha and beta), Presenilin (Psen1), |
| | nicastrin, (Ncstn), PEN-2, Nos1, Parp1, Nat1, Nat2); Trinucleotide Repeat Disorders |
| | (HTT (Huntington's Dx), SBMA/SMAX1/AR (Kennedy's Dx), FXN/X25 (Friedrich's |
| | Ataxia), ATX3 (Machado- Joseph's Dx), ATXN1 and ATXN2 (spinocerebellar ataxias), |
| | DMPK (myotonic dystrophy), Atrophin-1 and Atn1 (DRPLA Dx), CBP (Creb-BP - global |
| | instability), VLDLR (Alzheimer's), Atxn7, Atxn10). |
| Ocular | Age-related macular degeneration (Abcr, Ccl2, Cc2, cp (ceruloplasmin), Timp3, |
| diseases and | cathepsinD, Vldlr, Ccr2); Cataract (CRYAA, CRYA1, CRYBB2, CRYB2, PITX3, |
| disorders | BFSP2, CP49, CP47, CRYAA, CRYA1, PAX6, AN2, MGDA, CRYBA1, CRYB1, |
| | CRYGC, CRYG3, CCL, LIM2, MP19, CRYGD, CRYG4, BFSP2, CP49, CP47, HSF4, |
| | CTM, HSF4, CTM, MIP, AQP0, CRYAB, CRYA2, CTPP2, CRYBB1, CRYGD, |
| | CRYG4, CRYBB2, CRYB2, CRYGC, CRYG3, CCL, CRYAA, CRYA1, GJA8, CX50, |
| | CAE1, GJA3, CX46, CZP3, CAE3, CCM1, CAM, KRIT1); Corneal clouding and |
| | dystrophy (APOA1, TGFBI, CSD2, CDGG1, CSD, BIGH3, CDG2, TACSTD2, TROP2, |
| | M1S1, VSX1, RINX, PPCD, PPD, KTCN, COL8A2, FECD, PPCD2, PIP5K3, CFD); |
| | Cornea plana congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, NTG, NPG, |
| | CYP1B1, GLC3A); Leber congenital amaurosis (CRB1, RP12, CRX, CORD2, CRD, |
| | RPGRIP1, LCA6, CORD9, RPE65, RP20, AIPL1, LCA4, GUCY2D, GUC2D, LCA1, |
| | CORD6, RDH12, LCA3); Macular dystrophy (ELOVL4, ADMD, STGD2, STGD3, |
| | RDS, RP7, PRPH2, PRPH, AVMD, AOFMD, VMD2). |

TABLE 4
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; MAPK9; |
| | CDK2; PPP2CA; PIM1; ITGB7; YWHAZ; ILK; TP53; RAF1; IKBKG; RELB; |
| | DYRK1A; CDKN1A; ITGB1; MAP2K2; JAK1; AKT1; JAK2; PIK3R1; |
| | CHUK; PDPK1; PPP2R5C; CTNNB1; MAP2K1; NFKB1; PAK3; ITGB3; |
| | CCND1; GSK3A; FRAP1; SFN; ITGA2; TTK; CSNK1A1; BRAF; GSK3B; |
| | AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid | RAC1; TAF4B; EP300; SMAD2; TRAF6; PCAF; ELK1; MAPK1; SMAD3; |
| Receptor Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; RAC1; RAP1A; |
| Signaling | E1F4E; PRKCZ; NRP1; NTRK2; ARHGEF7; SMO; ROCK2; MAPK1; PGF; |
| | RAC2; PTPN11; GNAS; AKT2; PIK3CA; ERBB2; PRKCI; PTK2; CFL1; |
| | GNAQ; PIK3CB; CXCL12; PIK3C3; WNT11; PRKD1; GNB2L1; ABL1; |
| | MAPK3; ITGA1; KRAS; RHOA; PRKCD; PIK3C2A; ITGB7; GLI2; PXN; |
| | VASP; RAF1; FYN; ITGB1; MAP2K2; PAK4; ADAM17; AKT1; PIK3R1; |
| | GLI1; WNT5A; ADAM10; MAP2K1; PAK3; ITGB3; CDC42; VEGFA; |
| | ITGA2; EPHA8; CRKL; RND1; GSK3B; AKT3; PRKCA |
| Ephrin Receptor | PRKCE; ITGAM; ROCK1; ITGA5; CXCR4; IRAK1; PRKAA2; EIF2AK2; |
| Signaling | RAC1; RAP1A; GRK6; ROCK2; MAPK1; PGF; RAC2; PTPN11; GNAS; |
| | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRK1A; ITGB1; MAP2K2; PAK4, AKT1; JAK2; STAT3; ADAM10; |
| | MAP2K1; PAK3; ITGB3; CDC42; VEGFA; ITGA2; EPHA8; TTK; CSNK1A1; |
| | CRKL; BRAF; PTPN13; ATF4; AKT3; SGK |
| Actin Cytoskeleton | ACTN4; PRKCE; ITGAM; ROCK1; ITGA5; IRAK1; PRKAA2; EIF2AK2; |
| Signaling | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; MAPK9; |
| | CDK2; PIM1; PIK3C2A; ITGB7; PPP1CC; PXN; VIL2; RAF1; GSN; |
| | DYRK1A; ITGB1; MAP2K2; PAK4; PIP5K1A; PIK3R1; MAP2K1; PAK3; |
| | ITGB3; CDC42; APC; ITGA2; TTK; CSNK1A1; CRKL; BRAF; VAV3; SGK |
| Huntington's Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKC1; |
| | HSPA5; REST; GNAQ; PIK3CB; PIK3C3; MAPK8; IGF1R; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; ATF4; |
| | AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; PTPN11; AKT2; IKBKB; |
| Signaling | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; MAP3K7; |
| | MAP2K2; AKT1; PIK3R1; CHUK; MAP2K1; NFKB1; CDC42; GSK3A; |
| | FRAP1; BCL6; BCL10; JUN; GSK3B; ATF4; AKT3; VAV3; RPS6KB1 |
| Leukocyte | ACTN4; CD44; PRKCE; ITGAM; ROCK1; CXCR4; CYBA; RAC1; RAP1A; |
| Extravasation | PRKCZ; ROCK2; RAC2; PTPN11; MMP14; PIK3CA; PRKCI; PTK2; |
| Signaling | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; NOX1; |
| | PXN; VIL2; VASP; ITGB1; MAP2K2; CTNND1; PIK3R1; CTNNB1; CLDN1; |
| | CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; ARHGEF7; |
| | MAPK1; RAC2; CAPNS1; AKT2; CAPN2; PIK3CA; PTK2; PIK3CB; PIK3C3; |
| | MAPK8; CAV1; CAPN1; ABL1; MAPK3; ITGA1; KRAS; RHOA; SRC; |
| | PIK3C2A; ITGB7; PPP1CC; ILK; PXN; VASP; RAF1; FYN; ITGB1; |
| | MAP2K2; PAK4; AKT1; PIK3R1; TNK2; MAP2K1; PAK3; ITGB3; CDC42; |
| | RND3; ITGA2; CRKL; BRAF; GSK3B; AKT3 |
| Acute Phase | IRAK1; SOD2; MYD88; TRAF6; ELK1; MAPK1; PTPN11; AKT2; IKBKB; |
| Response | PIK3CA; FOS; NFKB2; MAP3K14; PIK3CB; MAPK8; RIPK1; MAPK3; |
| Signaling | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; MAP3K7; |
| | MAP2K2; AKT1; JAK2; PIK3R1; CHUK; STAT3; MAP2K1; NFKB1; FRAP1; |
| | CEBPB; JUN; AKT3; ILIR1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| | PIK3CA; CHEK1; TP53INP1; BCL2; PIK3CB; PIK3C3; MAPK8; THBS1; |
| | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAF1; CTNNB1; SIRT1; CCND1; PRKDC; ATM; SFN; |
| | CDKN2A; JUN; SNAI2; GSK3B; BAX; AKT3 |
| Aryl Hydrocarbon | HSPB1; EP300; FASN; TGM2; RXRA; MAPK1; NQO1; NCOR2; SP1; ARNT; |
| Receptor | CDKN1B; FOS; CHEK1; SMARCA4; NFKB2; MAPK8; ALDH1A1; ATR; |
| Signaling | E2F1; MAPK3; NRIP1; CHEK2; RELA; TP73; GSTP1; RB1; SRC; CDK2; |
| | AHR; NFE2L2; NCOA3; TP53; TNF; CDKN1A; NCOA2; APAF1; NFKB1; |
| | CCND1; ATM; ESR1; CDKN2A; MYC; JUN; ESR2; BAX; IL6; CYP1B1; |
| | HSP90AA1 |
| Xenobiotic | PRKCE; EP300; PRKCZ; RXRA; MAPK1; NQO1; NCOR2; PIK3CA; ARNT; |
| Metabolism | PRKCI; NFKB2; CAMK2A; PIK3CB; PPP2R1A; PIK3C3; MAPK8; PRKD1; |
| Signaling | ALDH1A1; MAPK3; NRIP1; KRAS; MAPK13; PRKCD; GSTP1; MAPK9; |
| | NOS2A; ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK | PRKCE; IRAK1; PRKAA2; EIF2AK2; RAC1; ELK1; GRK6; MAPK1; |
| Signaling | GADD45A; RAC2; PLK1; AKT2; PIK3CA; FADD; CDK8; PIK3CB; PIK3C3; |
| | MAPK8; RIPK1; GNB2L1; IRS1; MAPK3; MAPK10; DAXX; KRAS; |
| | PRKCD; PRKAA1; MAPK9; CDK2; PIM1; PIK3C2A; TRAF2; TP53; LCK; |
| | MAP3K7; DYRK1A; MAP2K2; PIK3R1; MAP2K1; PAK3; CDC42; JUN; |
| | TTK; CSNK1A1; CRKL; BRAF; SGK |
| PPAr/RXR | PRKAA2; EP300; INS; SMAD2; TRAF6; PPARA; FASN; RXRA; MAPK1; |
| Signaling | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; IL1R1; |
| | PRKCA; IL6; HSP90AA1; ADIPOQ |
| NF-KB Signaling | IRAK1; EIF2AK2; EP300; INS; MYD88; PRKCZ: TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4: |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin | ERBB4; PRKCE; ITGAM; ITGA5: PTEN; PRKCZ; ELK1; MAPK1; PTPN11; |
| Signaling | AKT2; EGFR; ERBB2; PRKCI; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPK1; MAP2K1; ITGB3; EREG; FRAP1; |
| | PSEN1; ITGA2; MYC; NRG1; CRKL; AKT3; PRKCA; HSP90AA1; RPS6KB1 |
| Wnt & Beta | CD44; EP300; LRP6; DVL3; CSNK1E; GJA1; SMO; AKT2; PIN1; CDH1; |
| catenin | BTRC; GNAQ; MARK2; PPP2R1A; WNT11; SRC; DKK1; PPP2CA; SOX6; |
| Signaling | SFRP2: ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin | PTEN; INS; EIF4E; PTPN1; PRKCZ; MAPK1; TSC1; PTPN11; AKT2; CBL; |
| Receptor | PIK3CA; PRKCI; PIK3CB; PIK3C3; MAPK8; IRS1; MAPK3; TSC2; KRAS; |
| Signaling | EIF4EBP1; SLC2A4; PIK3C2A; PPP1CC; INSR; RAF1; FYN; MAP2K2; |
| | JAK1; AKT1; JAK2; PIK3R1; PDPK1; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2: MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic | PRKCE; IRAK1; INS; MYD88; PRKCZ; TRAF6; PPARA; RXRA; IKBKB; |
| Cholestasis | PRKCI; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; IL8; |
| | CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKCI; |
| | PTK2; FOS; PIK3CB; PIK3C3; MAPK8; IGF1R; IRS1; MAPK3; IGFBP7; |
| | KRAS; PIK3C2A; YWHAZ; PXN; RAF1; CASP9; MAP2K2; AKT1; PIK3R1; |
| | PDPK1; MAP2K1; IGFBP2; SFN; JUN; CYR61; AKT3; FOXO1; SRF; CTGF; |
| | RPS6KB1 |
| NRF2-mediated | PRKCE; EP300; SOD2; PRKCZ; MAPK1; SQSTM1; NQO1; PIK3CA; PRKCI; |
| Oxidative Stress | FOS; PIK3CB; PIK3C3; MAPK8; PRKD1; MAPK3; KRAS; PRKCD; GSTP1; |
| Response | MAPK9; FTL; NFE2L2; PIK3C2A; MAPK14; RAF1; MAP3K7; CREBBP; |
| | MAP2K2; AKT1; PIK3R1; MAP2K1; PPIB; JUN; KEAP1; GSK3B; ATF4; |
| | PRKCA; EIF2AK3; HSP90AA1 |
| Hepatic | EDN1; IGF1; KDR; FLT1; SMAD2; FGFR1; MET; PGF; SMAD3; EGFR; |
| Fibrosis/Hepatic | FAS; CSF1; NFKB2; BCL2; MYH9; IGF1R; IL6R; RELA; TLR4; PDGFRB; |
| Stellate Cell | TNF; RELB; IL8; PDGFRA; NFKB1; TGFBR1; SMAD4; VEGFA; BAX; |
| Activation | IL1R1; CCL2; HGF; MMP1; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PPARGC1A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI | PRKCE; RAC1; PRKCZ; LYN; MAPK1; RAC2; PTPN11; AKT2; PIK3CA; |
| Signaling | SYK; PRKCI; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled | PRKCE; RAP1A; RGS16; MAPK1; GNAS; AKT2; IKBKB; PIK3CA; CREB1; |
| Receptor Signaling | GNAQ; NFKB2; CAMK2A; PIK3CB; PIK3C3; MAPK3; KRAS; RELA; SRC; |
| | PIK3C2A; RAF1; IKBKG; RELB; FYN; MAP2K2; AKT1; PIK3R1; CHUK; |
| | PDPK1; STAT3; MAP2K1; NFKB1; BRAF; ATF4; AKT3; PRKCA |
| Inositol Phosphate | PRKCE; IRAK1; PRKAA2; EIF2AK2; PTEN; GRK6; MAPK1; PLK1; AKT2; |
| Metabolism | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; MAPK8; |
| | CAV1; ABL1; MAPK3; KRAS; SRC; PIK3C2A; PDGFRB; RAF1; MAP2K2; |
| | JAK1; JAK2; PIK3R1; PDGFRA; STAT3; SPHK1; MAP2K1; MYC; JUN; |
| | CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer | PRKCE; RAC1; PRKCZ; MAPK1; RAC2; PTPN11; KIR2DL3; AKT2; |
| Cell | PIK3CA; SYK; PRKCI; PIK3CB; PIK3C3; PRKD1; MAPK3; KRAS; PRKCD; |
| Signaling | PTPN6; PIK3C2A; LCK; RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; CDKN1B; BTRC; ATR; ABL1; E2F1; |
| Checkpoint | HDAC2; HDAC7A; RB1; HDAC11; HDAC9; CDK2; E2F2; HDAC3; TP53; |
| Regulation | CDKN1A; CCND1; E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; NFKB2; PIK3CB; |
| Signaling | PIK3C3; MAPK8; MAPK3; KRAS; RELA, PIK3C2A; BTK; LCK; RAF1; |
| | IKBKG; RELB, FYN; MAP2K2; PIK3R1; CHUK; MAP2K1; NFKB1; ITK; |
| | BCL10; JUN; VAV3 |
| Death Receptor | CRADD; HSPB1; BID; BIRC4; TBK1; IKBKB; FADD; FAS; NFKB2; BCL2; |
| Signaling | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3; BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; PIM1; |
| | PIK3C2A; RAF1; MAP2K2; AKT1; JAK2; PIK3R1; STAT3; MAP2K1; |
| | CCND1; AKT3; STAT1 |
| Amyotrophic Lateral | BID; IGF1; RAC1; BIRC4; PGF; CAPNS1; CAPN2; PIK3CA; BCL2; PIK3CB; |
| Sclerosis Signaling | PIK3C3; BCL2L1; CAPN1; PIK3C2A; TP53; CASP9; PIK3R1; RAB5A; |
| | CASP1; APAF1; VEGFA; BIRC2; BAX; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STAT5A; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; PLK1; AKT2; CDK8; |
| Nicotinamide | MAPK8; MAPK3; PRKCD; PRKAA1; PBEF1; MAPK9; CDK2; PIM1; |
| Metabolism | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPP1CC; MAPK14; |
| | NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; RAF1; |
| | MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKCI; GNAQ; |
| Depression | PPP2R1A; IGF1R; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor | TAF4B; EP300; CARM1; PCAF; MAPK1; NCOR2; SMARCA4; MAPK3; |
| Signaling | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein | TRAF6; SMURF1; BIRC4; BRCA1; UCHL1; NEDD4; CBL; UBE2I; BTRC; |
| Ubiquitination | HSPA5; USP7; USP10; FBXW7; USP9X; STUB1; USP22; B2M; BIRC2; |
| Pathway | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKCI; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor | IRAK1; EIF2AK2; MYD88; TRAF6; PPARA; ELK1; IKBKB; FOS; NFKB2; |
| Signaling | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; ILIR1; SRF; STAT1 |
| Neurotrophin/TRK | NTRK2; MAPK1; PTPN11; PIK3CA; CREB1; FOS; PIK3CB; PIK3C3; |
| Signaling | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKCI; GNAQ; |
| Potentiation | CAMK2A; PRKD1; MAPK3; KRAS; PRKCD; PPP1CC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in | EDN1; PTEN; EP300; NQO1; UBE2I; CREB1; ARNT; HIF1A; SLC2A4; |
| the Cardiovascular | NOS3; TP53; LDHA; AKT1; ATM; VEGFA; JUN; ATF4; VHL; HSP90AA1 |
| System | |
| LPS/IL-1 Mediated | IRAK1; MYD88; TRAF6; PPARA; RXRA; ABCA1, MAPK8; ALDH1A1; |
| Inhibition of RXR | GSTP1; MAPK9; ABCB1; TRAF2; TLR4; TNF; MAP3K7; NR1H2; SREBF1; |
| Function | JUN; IL1R1 |
| LXR/RXR Activation | FASN; RXRA; NCOR2; ABCA1; NFKB2; IRF3; RELA; NOS2A; TLR4; TNF; |
| | RELB; LDLR; NR1H2; NFKB1; SREBF1; IL1R1; CCL2; IL6; MMP9 |
| Amyloid Processing | PRKCE; CSNK1E; MAPK1; CAPNS1; AKT2; CAPN2; CAPN1; MAPK3; |
| | MAPK13; MAPT; MAPK14; AKT1; PSEN1; CSNK1A1; GSK3B; AKT3; APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling | KDR; FLT1; PGF; AKT2; PIK3CA; PIK3CB; PIK3C3; CAV1; PRKCD; NOS3; |
| in the Cardiovascular | PIK3C2A; AKT1; PIK3R1; VEGFA; AKT3; HSP90AA1 |
| System | |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated | RAP1A; MAPK1; GNAS; CREB1; CAMK2A; MAPK3; SRC; RAF1; |
| Signaling | MAP2K2; STAT3; MAP2K1; BRAF; ATF4 |
| Mitochondrial | SOD2; MAPK8; CASP8; MAPK10; MAPK9; CASP9; PARK7; PSEN1; |
| Dysfunction | PARK2; APP; CASP3 |
| Notch Signaling | HES1; JAG1; NUMB; NOTCH4; ADAM17; NOTCH2; PSEN1; NOTCH3; |
| | NOTCH1; DLL4 |
| Endoplasmic Reticulum | HSPA5; MAPK8; XBP1; TRAF2; ATF6; CASP9; ATF4; EIF2AK3; CASP3 |
| Stress Pathway | |
| Pyrimidine Metabolism | NME2; AICDA; RRM2; EIF2AK4; ENTPD1; RRM2B; NT5E; POLD1; NME1 |
| Parkinson's Signaling | UCHL1; MAPK8; MAPK13; MAPK14; CASP9; PARK7; PARK2; CASP3 |
| Cardiac & Beta | GNAS; GNAQ; PPP2R1A; GNB2L1; PPP2CA; PPP1CC; PPP2R5C |
| Adrenergic Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine | PRDX6; PRDX1; TYR |
| and Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; Trpa1; Pomc; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; Wnt3a; Wnt4; |
| Neurology | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4fl or Brn3a); Numb; ReLN |

Chimeric Antigen Receptors and Engineered T Cell Receptors

The payload protein(s) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracelluar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GaLNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Pharmaceutically Acceptable Compositions and Methods

Disclosed herein include pharmaceutical compositions. In some embodiments, the pharmaceutical composition comprises: a composition provided herein (e.g., a nucleic acid composition, a population of engineered cells), wherein the pharmaceutical composition further comprises one or more pharmaceutically acceptable carriers, diluents and/or excipients. There are provided, in some embodiments, methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., engineered cells, nucleic acid composition) which can be combined with a carrier material to produce a single dosage form will vary depending upon the host being treated, the particular mode of administration. The amount of active ingredient, which can be combined with a carrier material to produce a single dosage form will generally be that amount of the nucleic acid composition and/or engineered cells which produces a therapeutic effect. Generally, out of one hundred percent, this amount will range from about 1% to about 99% of active ingredient, preferably from about 5% to about 70%, most preferably from about 10% to about 30%.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the engineered cells disclosed herein, thereby treating or preventing the disease or disorder in the subject. In some embodiments, administering comprises: (i) isolating one or more cells from the subject; (ii) contacting (e.g., transfecting) said one or more cells with a nucleic acid composition disclosed herein, thereby generating engineered cells; and (iii) administering the one or more engineered cells into a subject after the contacting step. The method can comprise: administering to the subject an effective amount of a pro-death agent, or any combination thereof. The engineered sends can be configured to travel to and/or accumulate at a target site of a subject. In some embodiments, nucleic acid composition(s) are administered to a subject to generate engineered cells in vivo. Alternatively, in some embodiments, engineered cells are generated (e.g., by incorporating the nucleic acid composition(s) provided herein) outside the body of the subject and are subsequently administrated to the subject.

The disclosed engineered cells described herein may be included in a composition for therapy. In some embodiments, the composition comprises a population of disclosed engineered cells. The composition may include a pharmaceutical composition and further include a pharmaceutically acceptable carrier. A therapeutically effective amount of the pharmaceutical composition comprising the disclosed engineered cells may be administered. The cells provided herein may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating an engineered population of cells. The disclosed engineered cells can be administered to a mammalian recipient to provide a therapeutic benefit. The mammalian recipient may be a human and the disclosed engineered cells can be autologous with respect to the recipient. Alternatively, the disclosed engineered cells can be allogeneic, syngeneic or xenogeneic with respect to the recipient.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The disclosed engineered cells can be administered at a therapeutically effective amount. For example, a therapeutically effective amount of the disclosed engineered cells can be at least about 10² cells, at least about 10³ cells, at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the disclosed engineered cells is about 10⁴ cells, about 10⁵ cells, about 10⁶ cells, about 10⁷ cells, or about 10⁸ cells. In one particular embodiment, the therapeutically effective amount of the disclosed engineered cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, a solid tumor, or any combination thereof. The disease or disorder can be an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enterovirus (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen. The disease associated with expression of a tumor antigen-associated can be selected from the group consisting of a proliferative disease, a precancerous condition, a cancer, and a non-cancer related indication associated with expression of the tumor antigen. The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

### EXAMPLES

Some aspects of the embodiments discussed above are disclosed in further detail in the following examples, which are not in any way intended to limit the scope of the present disclosure.

### Example 1

A Synthetic Protein-Level Neural Network in Mammalian Cells Introduction

Cells are classification machines. Using circuits of interacting genes and proteins, they make qualitatively distinct decisions in response to the levels or dynamics of multiple input signals. For example, p53 functions as a tumor suppressor by classifying the types and levels of stress the cell encounters, and inducing senescence or cell death in response. In the context of development, cells in the neural tube take on specific progenitor fates by classifying the levels of BMP and Hedgehog signaling. In the human immune system, the classification of multiple cytokine inputs can control the fate of a T cell. The ability to program synthetic signal classification systems could facilitate engineered gene and cell therapies by allowing cells to robustly distinguish diseased and normal cell states. For this reason, a major goal of synthetic biology has been to design synthetic classification circuits that could function in living cells, respond to different types of signals as inputs and control cellular functions as outputs.

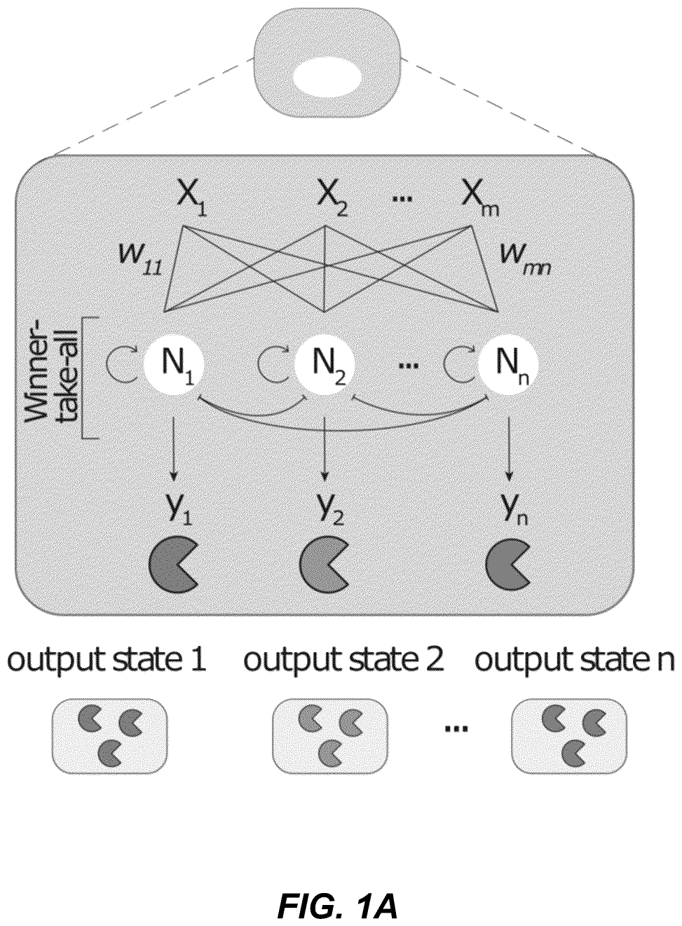

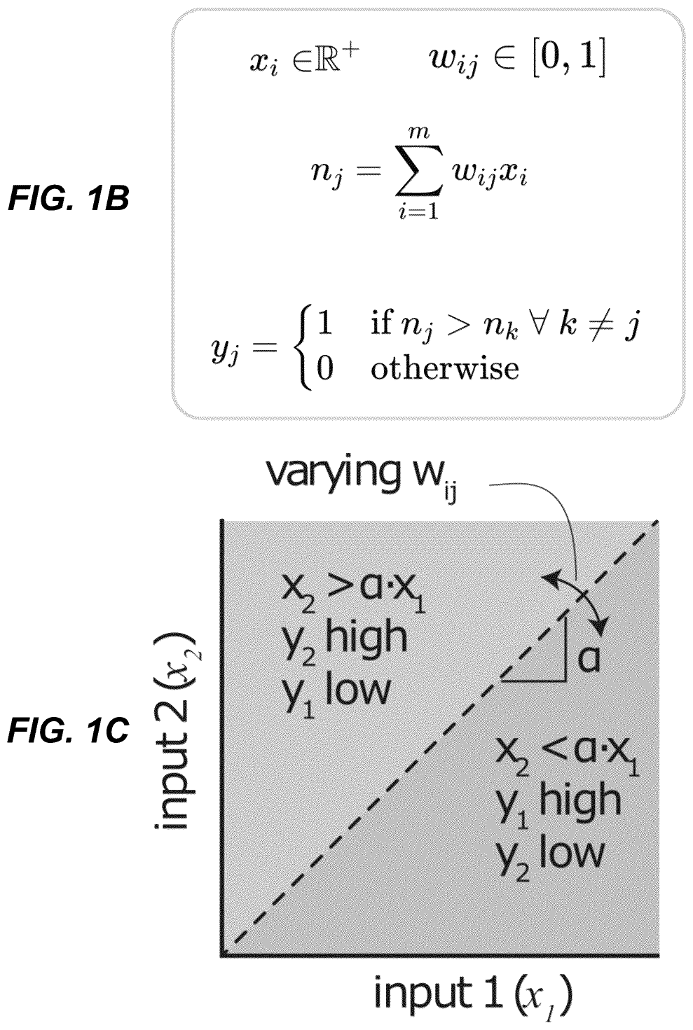

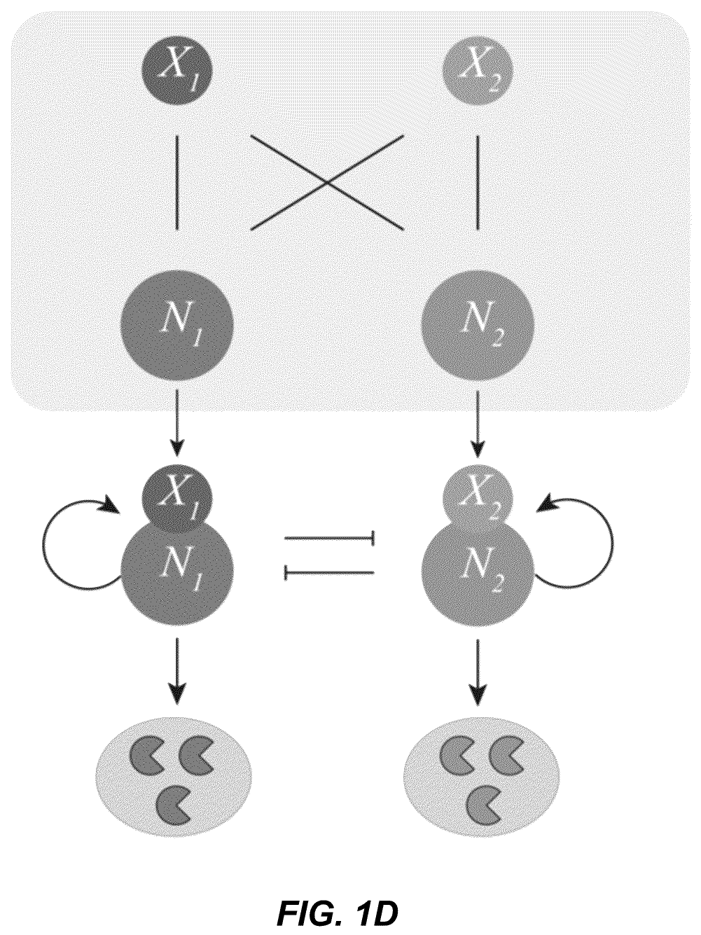

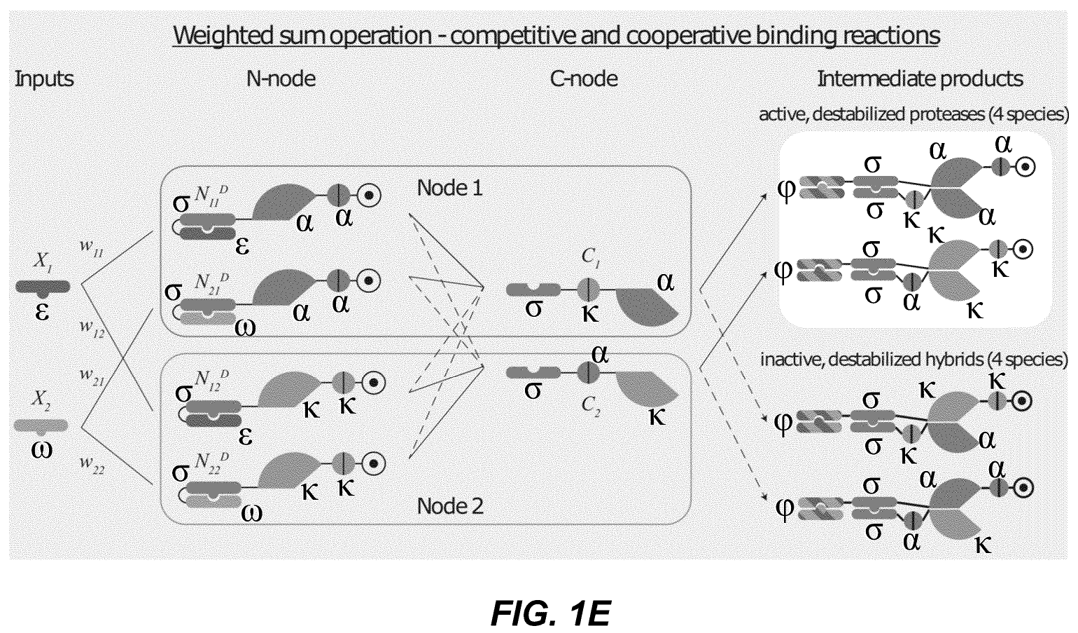

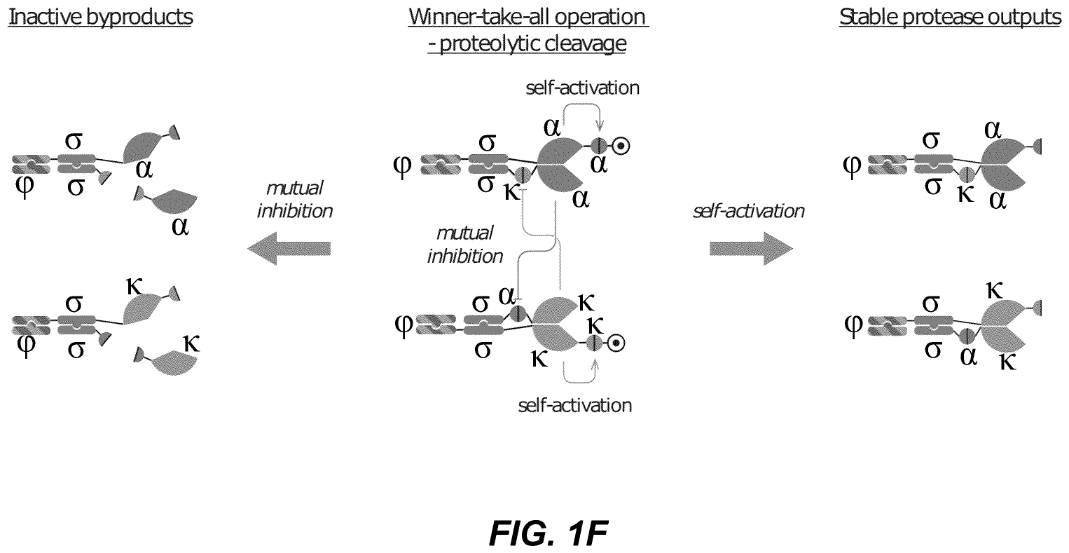

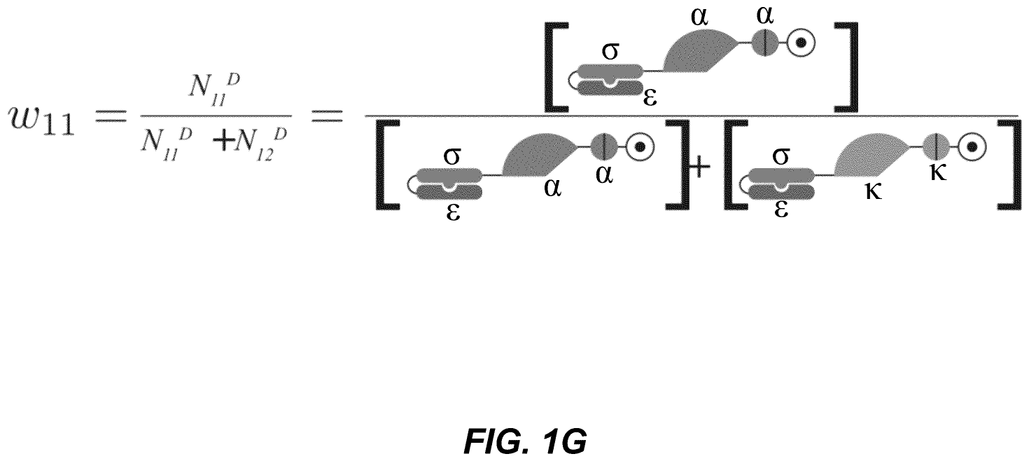

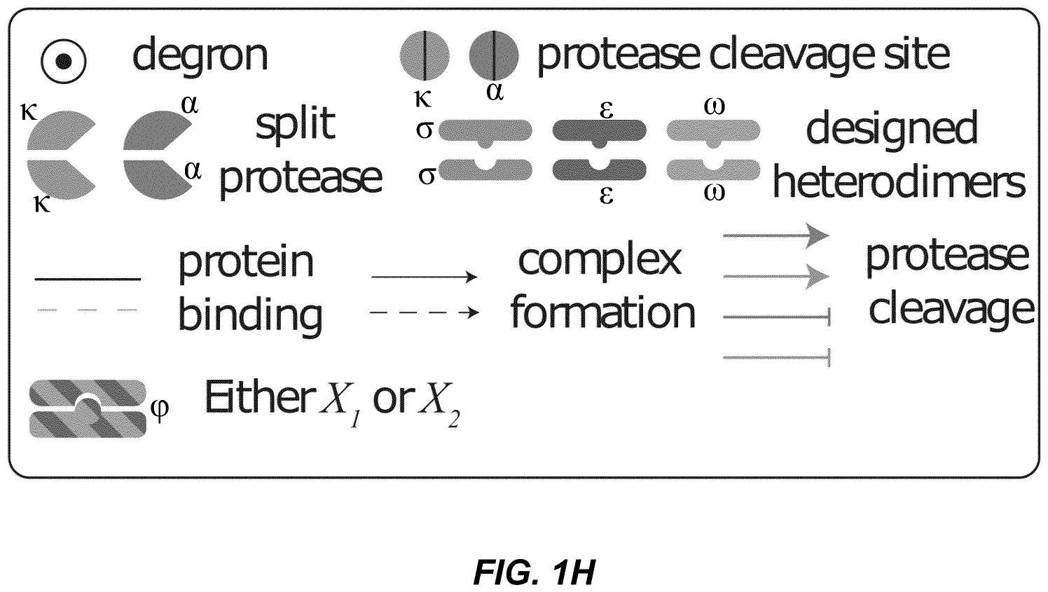

One of the most powerful circuit architectures for classification is the winner-take-all neural network. In these systems, an output neuron is ON if and only if the weighted sum of its inputs exceeds that of all other neurons in the output layer (FIGS. 1A-1B). This architecture has several benefits. First, it offers a compact mechanism for signal classification, requiring only a single layer neural network. Second, it ensures that outputs are all-or-none (FIG. 1B). Finally, it allows one to alter the decision boundary (e.g., an area of ambiguity in the classification landscape) simply by tuning weights (FIG. 1C).

Previous theoretical work suggested specific schemes for engineering biochemical neural computation systems. Experimentally, efforts to build synthetic classification systems have resulted in DNA classifiers in test tubes, and miRNA-based classifiers in mammalian cells. In contrast, a protein level classifier would offer several advantages: it could be expressed transiently in cells, interface directly with endogenous inputs and outputs, trigger different output pathways depending on input state, and should in principle work in diverse cell types without relying on endogenous transcriptional regulations. More generally, a protein-based neural network would also test the ability to construct sophisticated computational devices out of interacting proteins.

A key challenge in designing a protein-based neural network has been the increased difficulty of programming specific binding interactions and conditional activity using proteins compared to nucleic acids. Sets of modular protein interaction domains have been developed, including de novo designed heterodimers (DHDs). Additionally, multiple groups have used conditional reconstitution of split viral proteases as a way to control protein activities. Herein is provided a protein based winner-take-all comparator circuit using DHDs and split proteases that senses and processes input signals. This circuit accurately compared the relative levels of two inputs in living mammalian cells, with a tunable decision boundary. This synthetic protein neural network provides a foundation for rationally engineering protein classification in living cells.

### Results

System Design

Winner-take-all dynamics can be achieved with molecular components that exhibit three key features (FIG. 1D): First, they should respond to input molecules with tunable strengths, analogous to weights in neural networks, to enable weighted summation of inputs. Second, they should be capable of mutual inhibition, allowing the elimination of less abundant species. Third, they should be able to self-activate, so that surviving species can amplify their own activity.

Fusion proteins were created that possess these features by genetically combining de novo designed protein heterodimers (DHDs), domains from viral proteases such as Tobacco Etch Virus (TEV) and Tobacco Vein Mottling Virus (TVMV) (FIG. 1E), and the dihydrofolate reductase (DHFR) degron. Briefly, the proteases were split into two inactive domains, whose reconstitution into an active protease can be controlled by attached DHD domains. The intermolecular dimerization of these DHD domains can be regulated by input DHDs that disrupt a competing inhibitory self-caging interaction (FIG. 1E). The strength of this input binding interaction, analogous to a weight in a neural network, is tunable by varying the concentrations of DHD-protease fusions (FIG. 1G). Once reconstituted, proteases can inactivate one another by cleaving off attached dimerization domains, achieving mutual inhibition (FIG. 1F). Finally, the same proteases can also self-activate by cleaving off degrons that would otherwise destabilize them (FIG. 1F). Together, these components generate the key interactions required for constructing a set of protein-based winner-take-all neural networks termed herein perceptein networks, in loose analogy with the perceptron, a foundational artificial neural network architecture.

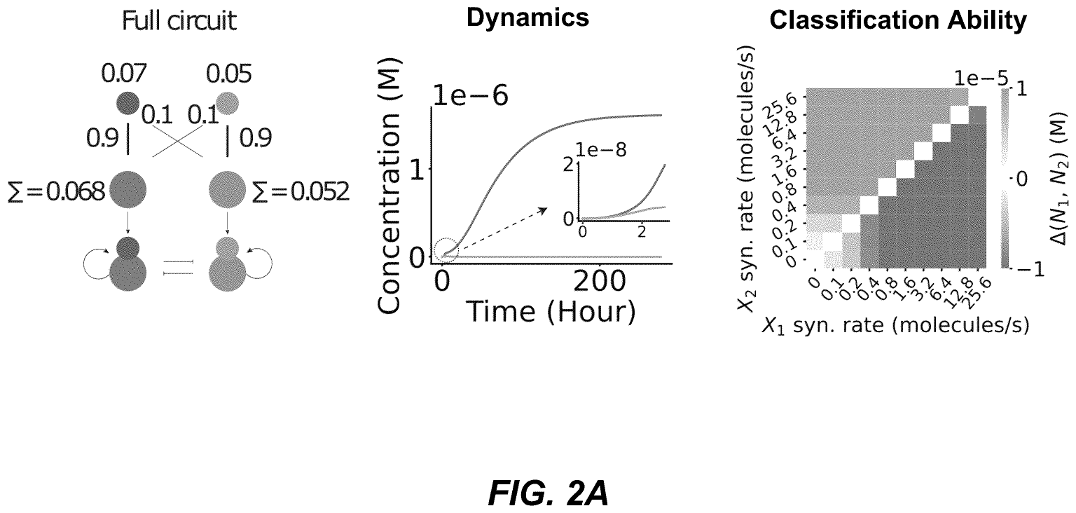

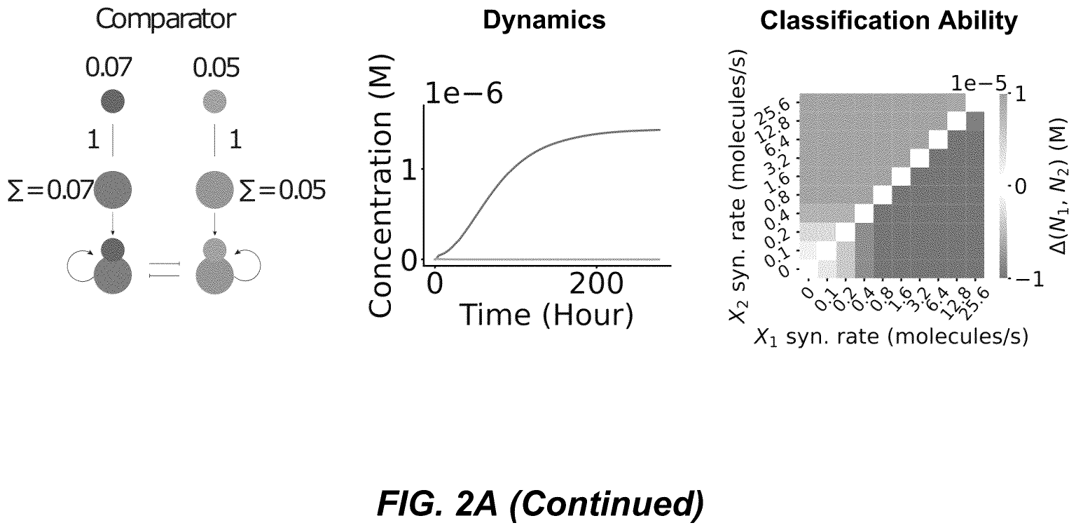

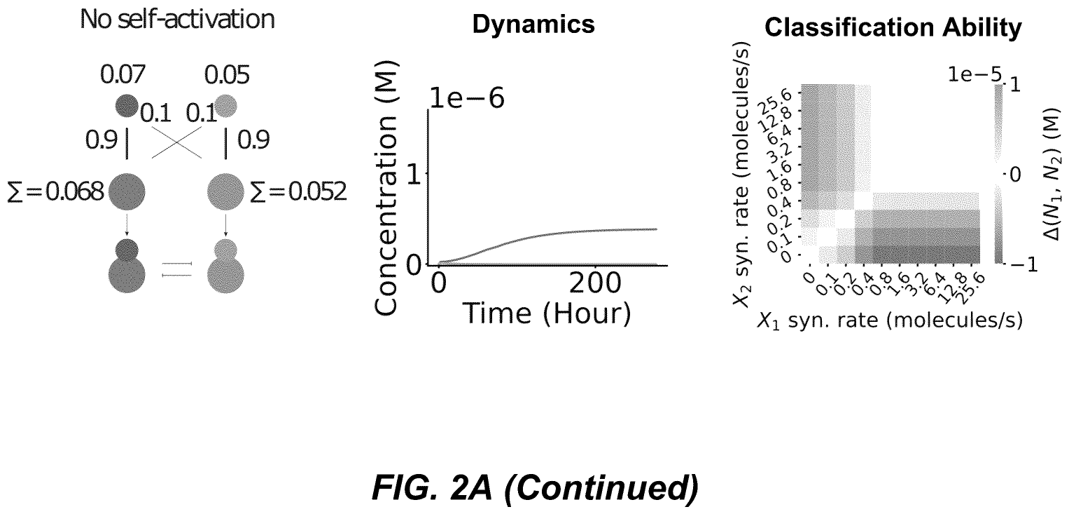

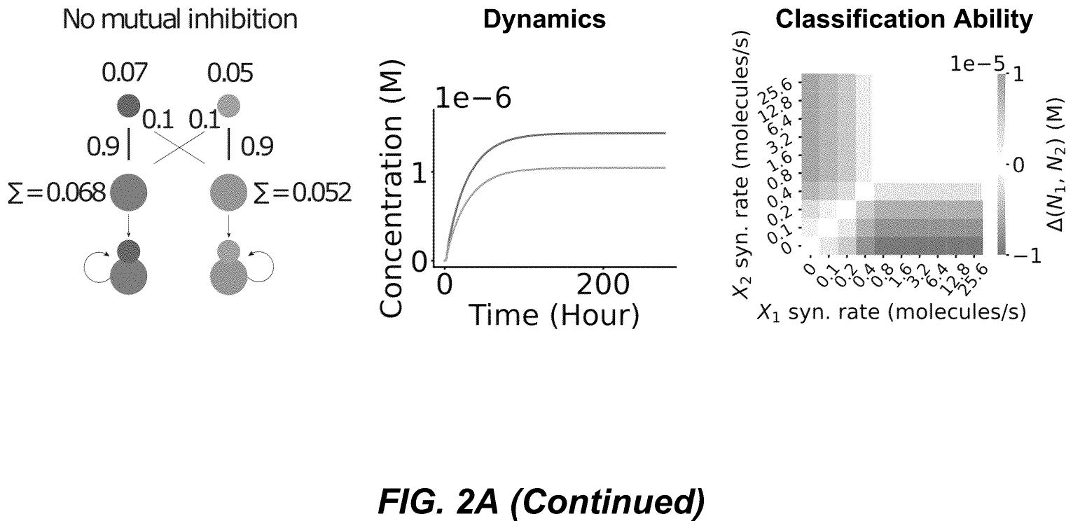

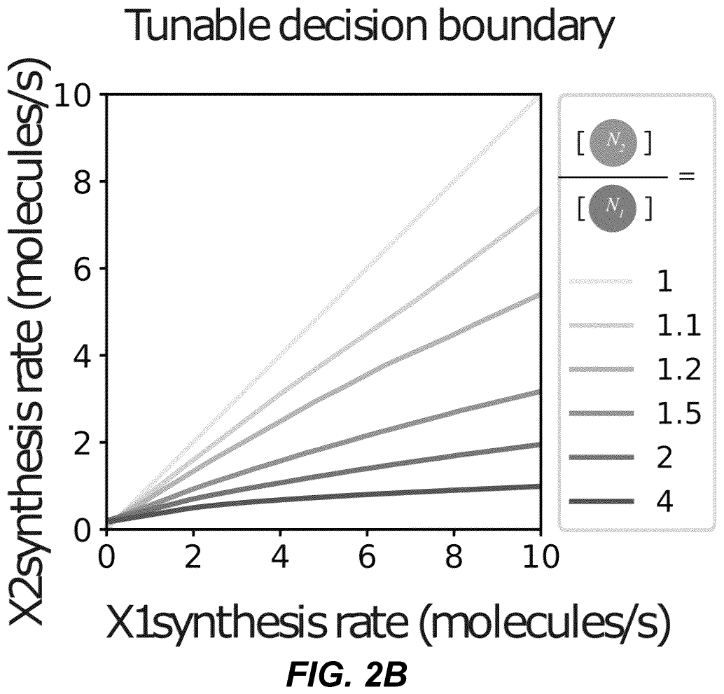

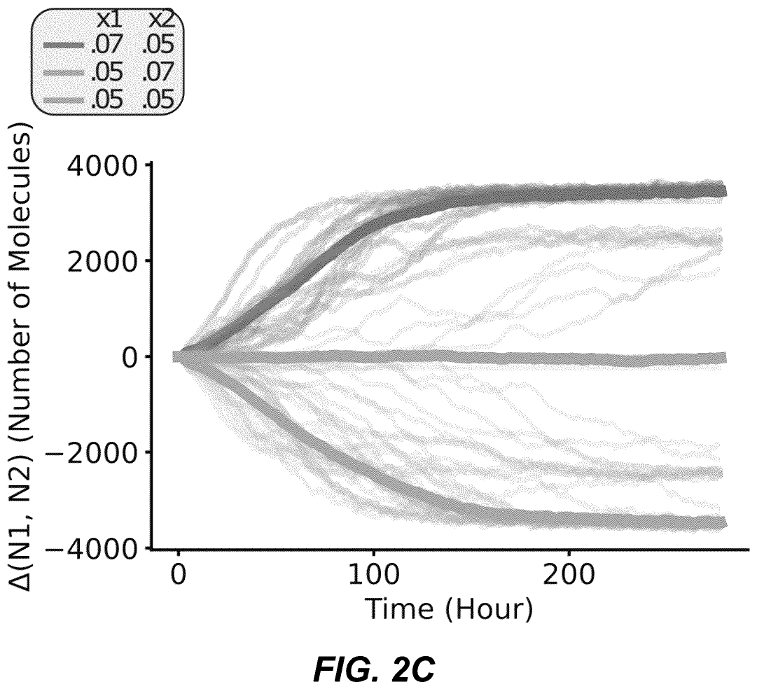

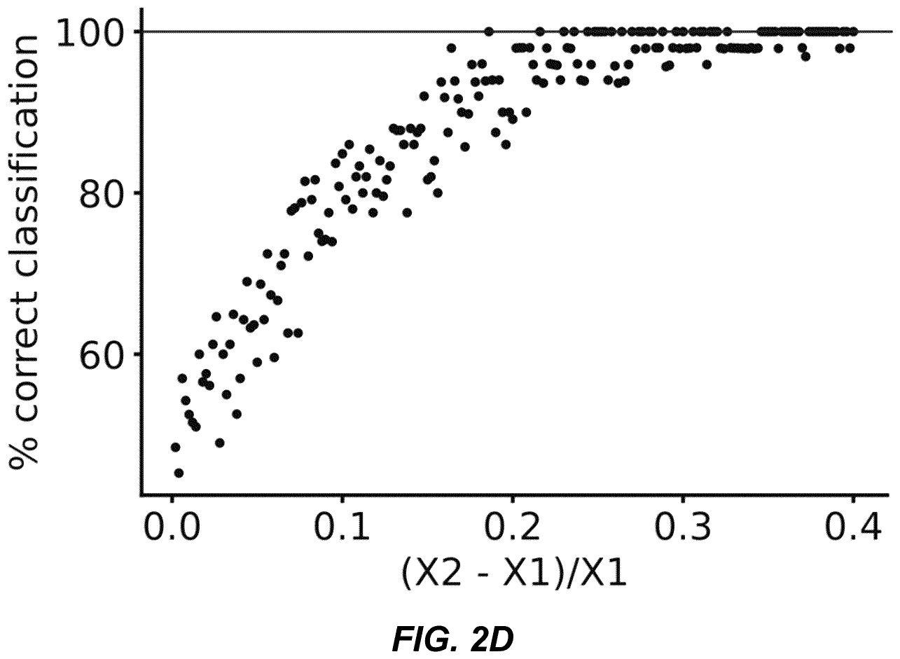

In the language of neural networks, the perceptein network comprises a layer of inputs (DHD input proteins, Xᵢ), as well as a layer of nodes (Nᵢ) that process information from inputs and couple to outputs (FIG. 2). In this molecular implementation, each node consists of two parts, labeled N-nodes and C-nodes. The N-nodes are composed of Nⱼₖ^{D} or Nⱼₖ, consisting of N-terminal protease halves fused to DHDs, with (Nⱼₖ^{D}) or without (Nⱼₖ) an attached degradation tag. The C-nodes, labeled Cₗ, contain C-terminal protease halves fused to DHDs (FIG. 1E). In the simple case of two inputs and two outputs, all indices range from 1 to 2, such that the whole system consists of two Xᵢ, four Nⱼₖ^{D}, and two Cₗ components, or eight proteins altogether. Throughout the text, Nᵢ refers to the node consisting of N-node and C-node proteins, while Nⱼₖ^{D} and Nⱼₖ refers to individual N-node proteins.

The disclosed design enables weighted summation of inputs. The i^{th} input species, Xᵢ, is partitioned among different potential binding partners Nⱼₖ^{D} (for which i=j) in proportion to their relative abundances. The effective weights, wᵢⱼ, are thus determined by the abundances of the corresponding Nⱼₖ^{D} components. For example, in the case of 2 inputs and 2 outputs, w₁₁ is defined as the concentration of N₁₁^{D} divided by the sum of the concentrations of N₁₁^{D} and N₁₂^{D} (FIG. 1G). Binding to an input uncages the DHD domain fused to Nⱼₖ^{D} components, exposing the Nⱼₖ^{D} binding domain (gray), which can interact with Cₗ domains to produce either a functional protease (if k=l) or a non-functional hybrid of mismatched protease halves (if k≠l) (FIG. 1E). Each input can, in this way, generate both functional reconstituted proteases as well as non-functional hybrids. Input summation occurs because the total set of functional reconstituted proteases of a given type in general includes contributions from all inputs.

The design also enables self-activation and mutual inhibition. To achieve self-activation, functional proteases can further activate their own activity by cleaving off a degron that is present on the Nⱼₖ^{D} components of the same protease type, which would otherwise cause rapid protease degradation (FIG. 1F). The functional reconstituted proteases of each type can also inactivate other functional proteases by cleaving off DHDs on their Cₗ domains (FIG. 1F).

Modeling and Simulation

To understand whether this design could produce winner-take-all behavior within physiologically relevant parameter regimes, its response to a matrix of input values (Xᵢ concentrations) was first simulated. Briefly, inputs Xᵢ bind cooperatively to Nⱼₖ^{D} and Cₗ to form trimeric complexes that reconstitute either functional proteases or nonfunctional hybrids with mismatched protease halves. To model this cooperative binding process, the process of trimer formation was divided into two steps. First, Xᵢ binds to Nᵢⱼ^{D} to form an unstable dimer with fast off rates. This step also exposes the binding site for Cₗ. In the second step, this dimer binds to Cₗ to form stable trimeric complexes with slower off rates. Complexes with matching protease halves are assumed to reconstitute active protease. Each reconstituted protease can cleave its various cognate target sites on other protein components. All the while, all protein building blocks are continuously synthesized at constant rates and degraded at different rates depending on whether they contain a degron. Finally, physiologically reasonable values for protein synthesis and degradation rates, protease catalytic rates, and other biochemical parameters was estimated using references in the BioNumbers database (Table 5).

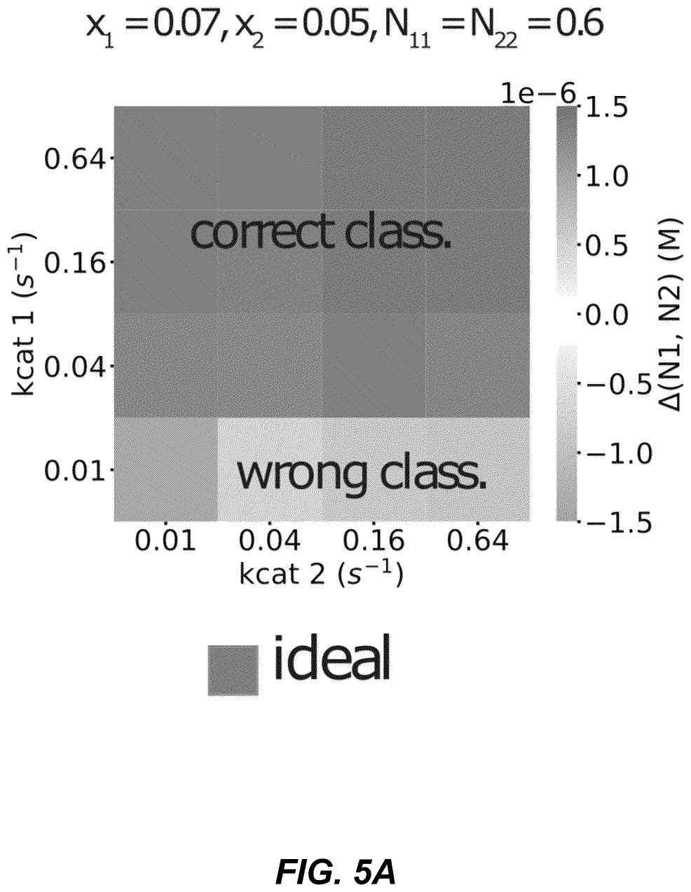

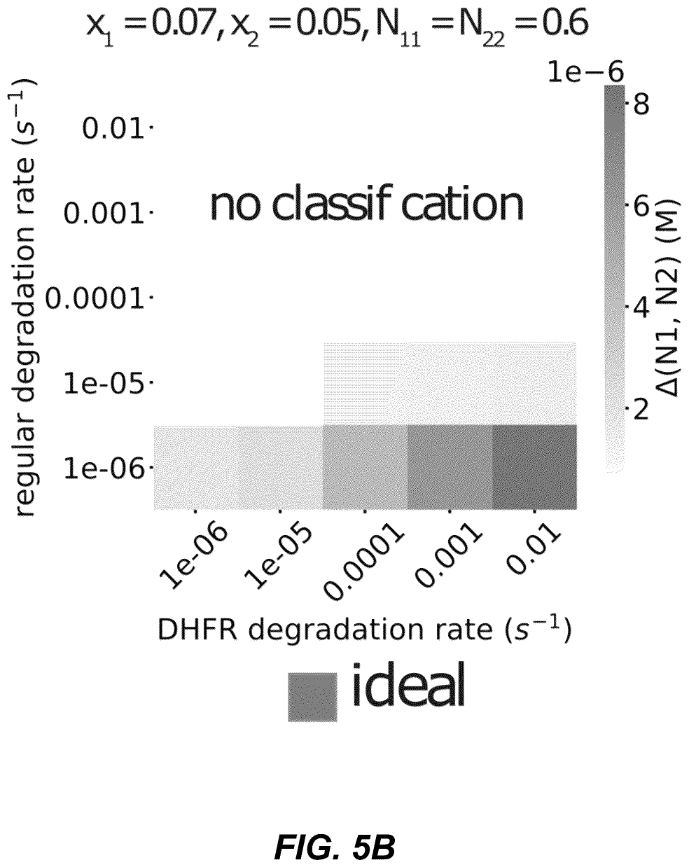

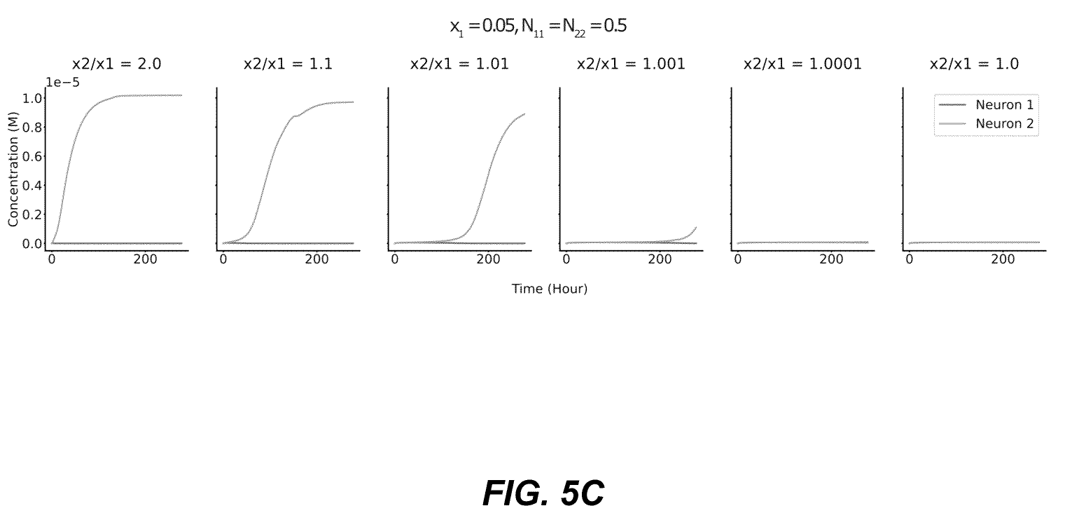

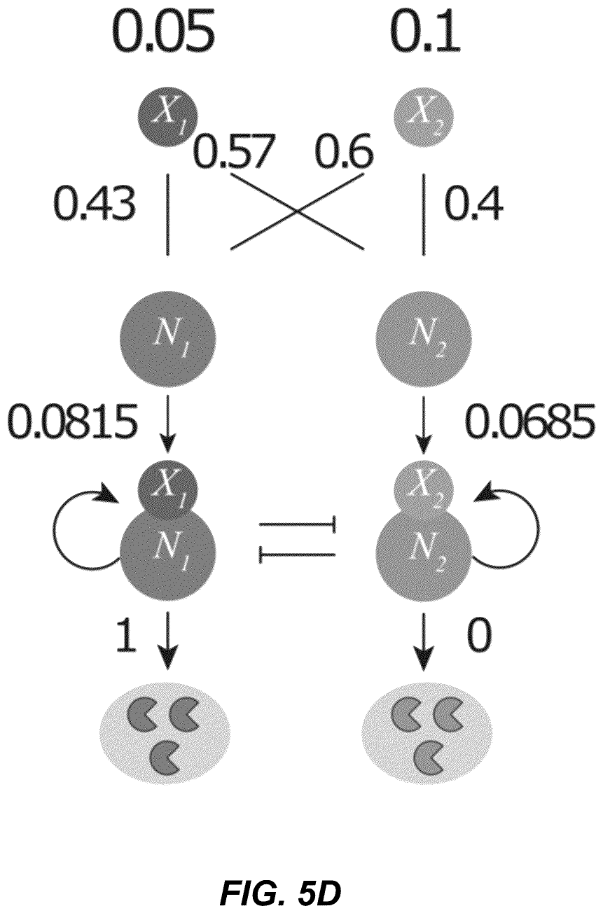

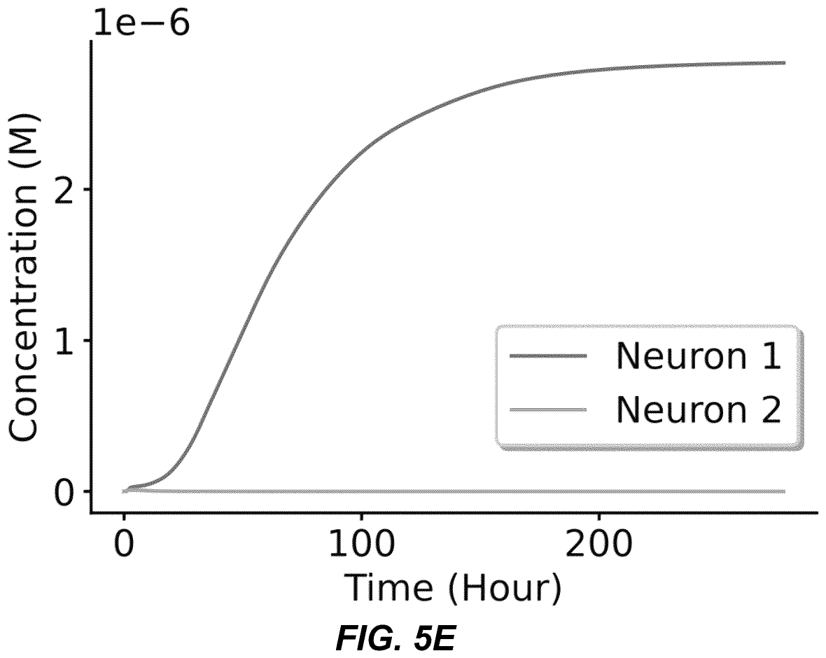

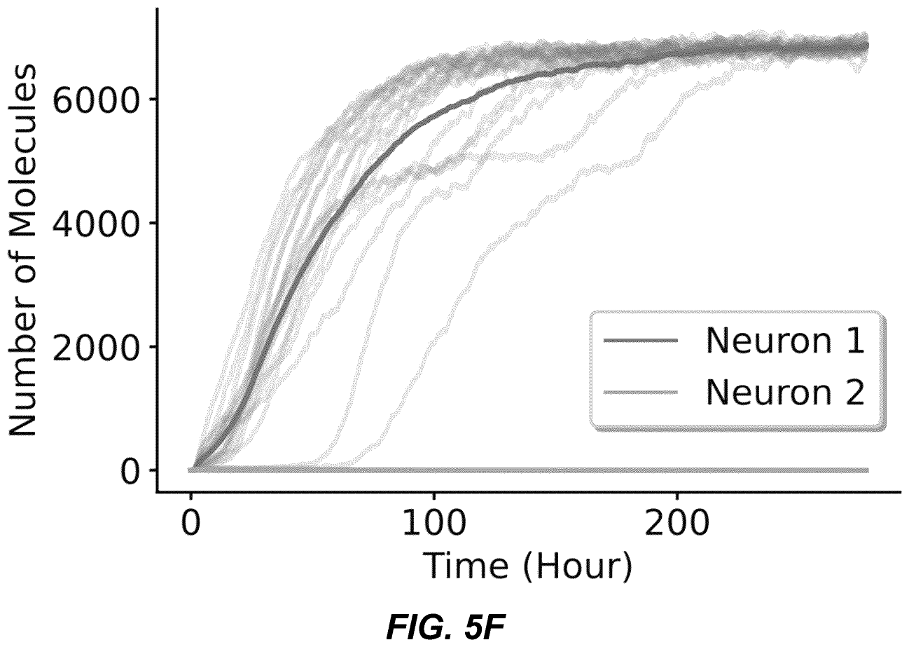

With these parameter values, the simulated circuit exhibited the desired winner-take-all classification behavior (FIG. 2A, first row). When X₁ exceeded X₂, the N₁ protease activation approached its maximum possible level over timescales of ˜100h. However, the decision appeared to be made much earlier, as large fold differences between N₁ and N₂ were apparent within 3 hours (FIG. 2A, first row inset). Simulations further revealed that winner-take-all classification functions across a broad range of absolute concentrations for X₁ and X₂, and remained accurate even for differences as small as 10% between the two ligand concentrations (FIG. 5C).

To understand which features of the circuit are necessary or sufficient for classification, circuit variants lacking cross-activation by inputs (FIG. 2A, second row), self-activation (FIG. 2A, third row), or mutual inhibition (FIG. 2A, fourth row) were also analyzed. Removing cross interactions (e.g., removing N₁₂^{D} and N₂₁^{D}) produced a simplified circuit design termed herein the comparator (FIG. 2A, second row) that is still capable of winner-take-all classification. Removing self-activation retains all-or-none behavior at lower input levels, but at a lower dynamic range of outputs (FIG. 2A, third row). At higher input levels, it loses classification ability altogether. By contrast, removing mutual repression accelerated the response of the circuit but eliminated the all-or-none output behavior, and also lost classification ability at higher input levels (FIG. 2A, bottom row). Therefore, in some embodiments, and without being bound by any particular theory, both self-activation and mutual inhibition are indispensable for winner-take-all computation in this architecture.

Sensitivity to parameter values within the circuit was next analyzed. Protease catalytic rates and protein degradation rates both had strong effects on classification accuracy (FIGS. 5A-5B). Lower values of k_{cat} led to reduced cleavage, hindering self-activation and cross-inhibition and therefore decreasing classification performance. However, as long as k_{cat} values for both proteases were above 0.04 sec⁻¹, the circuit operated correctly, identifying the larger of the two inputs (FIG. 5A). Additionally, the winner-take-all behavior was more pronounced at higher degradation rates, corresponding to stronger degrons in the experimental circuit (FIG. 5B, x-axis). By suppressing background protease activity, degradation amplifies the effects of self-activation.

In general, different inputs can vary over different ranges. An ideal circuit would allow one to tune the decision boundary to match the scales of the inputs. In these circuits, the decision boundary can in fact be tuned by varying weights, represented here as component concentrations. In the comparator, when N₁₁^{D} and N₂₂^{D} are fixed at the same level, the circuit compares input levels without bias. On the other hand, varying the relative abundances of N₁₁^{D} and N₂₂^{D} allows the construction of biased comparators, where N₂ is ON only when X₂>X₁·α, where α depends on the relative levels of N₁₁^{D} and N₂₂^{D}. Simulations confirmed that the circuit could implement such tunable decision boundaries (FIG. 2B).

Within cells, stochastic fluctuations can strongly impact circuit behaviors. Stochastic simulations of the circuit revealed that both the full network and the comparator circuits could function accurately despite such noise (FIG. 2C, FIGS. 5D-5F). Even with a difference in input values of only 40% (e.g. X₁=0.05, X₂=0.07), no “reversal” events were observed, as shown by individual traces (red and orange lines, FIG. 2C). On the other hand, when starting from equal inputs (X₁=X₂=0.05), individual trajectories converged over a slower timescale to either of the two output states, with an average response of neither (dark gray line, FIG. 2C), showing that the output is bistable. To analyze the sensitivity to input differences in the presence of noise, X₁−X₂ was varied and the fraction of correct decisions was analyzed. This analysis suggested that differences of only 20% were sufficient for accurate classification more than 95% of the time (FIG. 2D).

These results suggest that the perceptein architecture should function across a broad, biologically plausible range of parameter values. On the other hand, the perceptein produces an enormous number of distinct molecular species. Even in the smallest implementation, involving two inputs and two neurons (FIG. 2A, first row), starting from just 8 protein species, the system generates (by cleavage and protein complex formation) 158 unique proteins and protein complexes, that participate in 310 distinct chemical reactions, including protein binding, synthesis, degradation, and protease cleavage. Modeling these reactions required a set of 158 ordinary differential equations containing 1238 terms. This complexity, which exceeds that of most previous synthetic biological circuits, provokes the question of whether the system could actually function in mammalian cells.

Experimental Validation

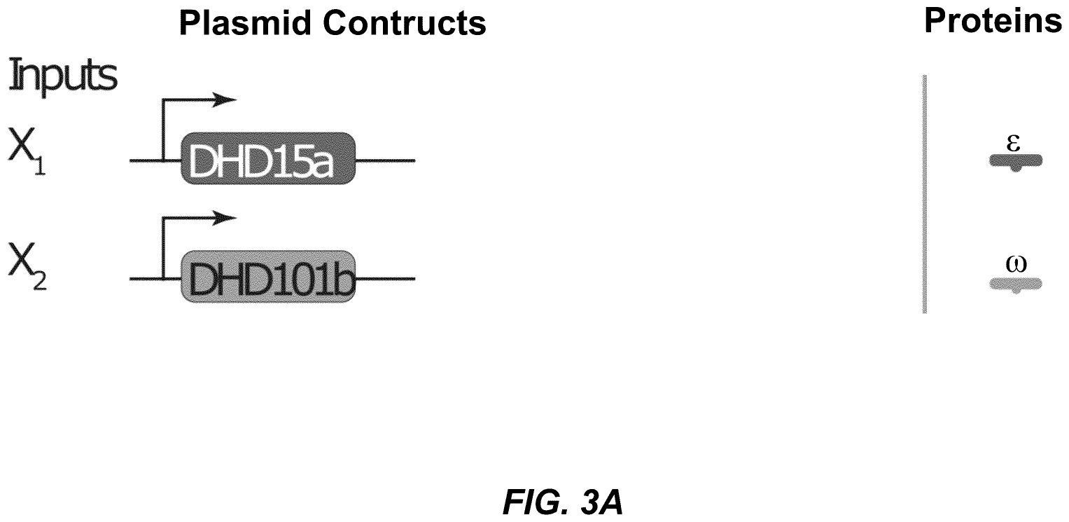

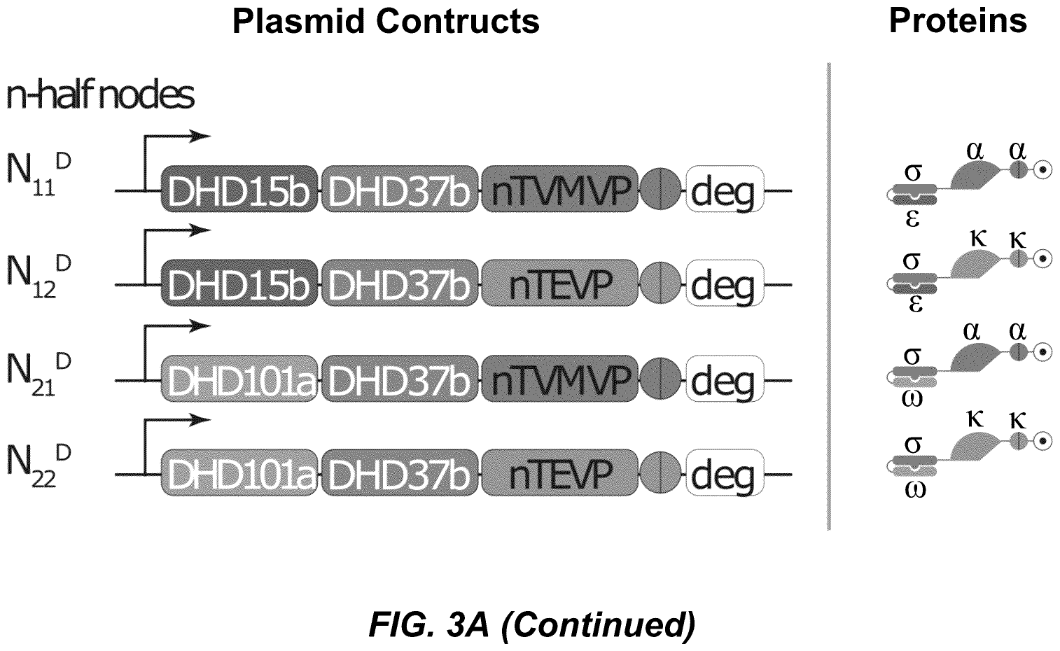

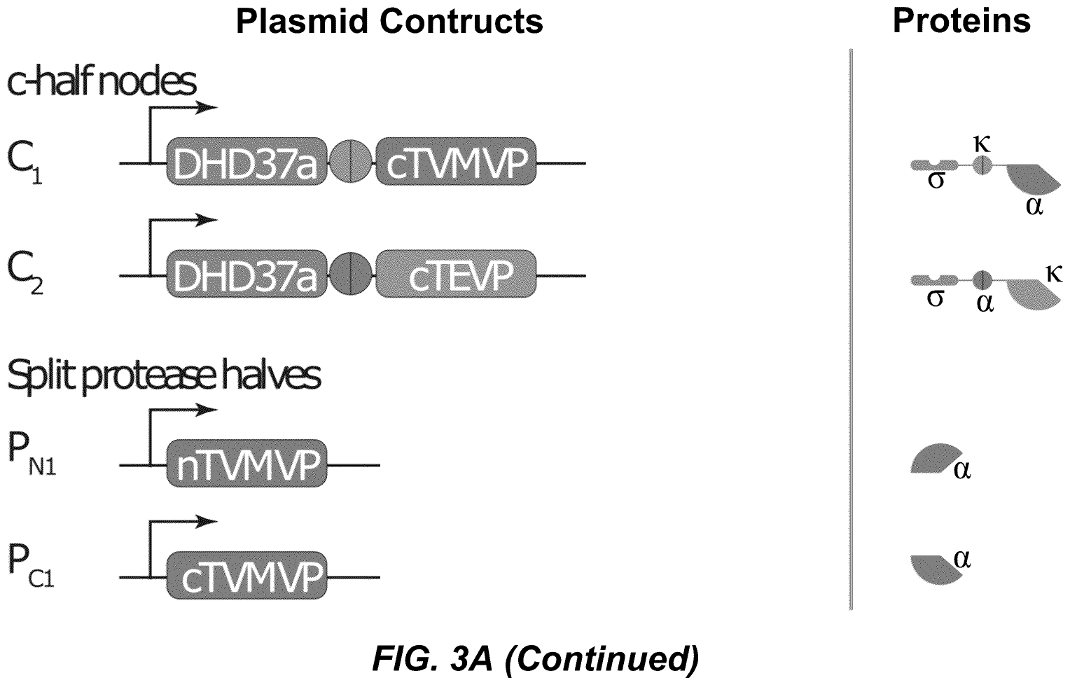

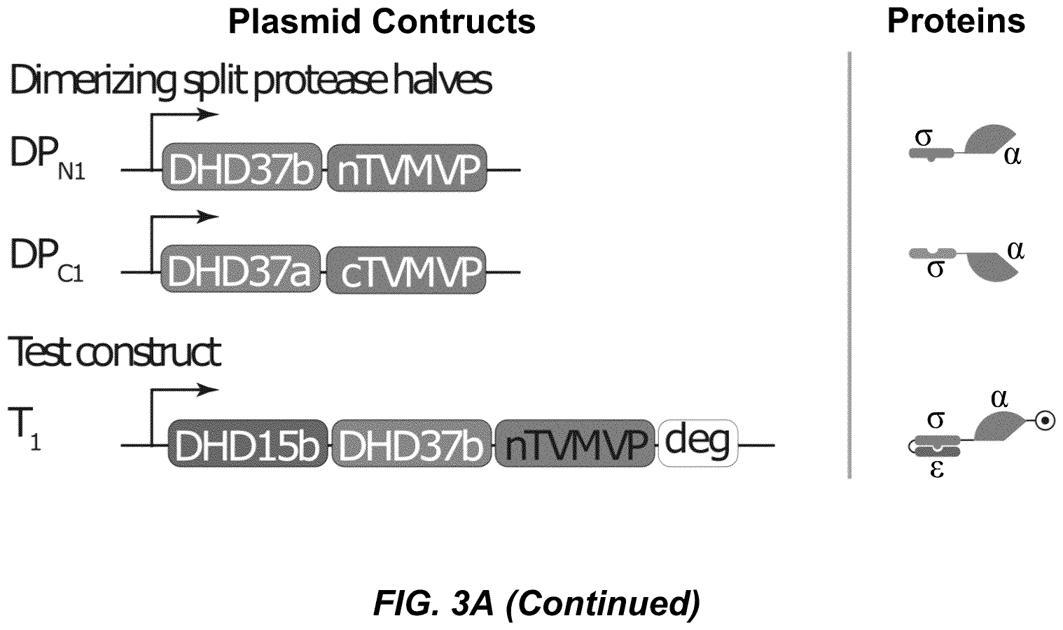

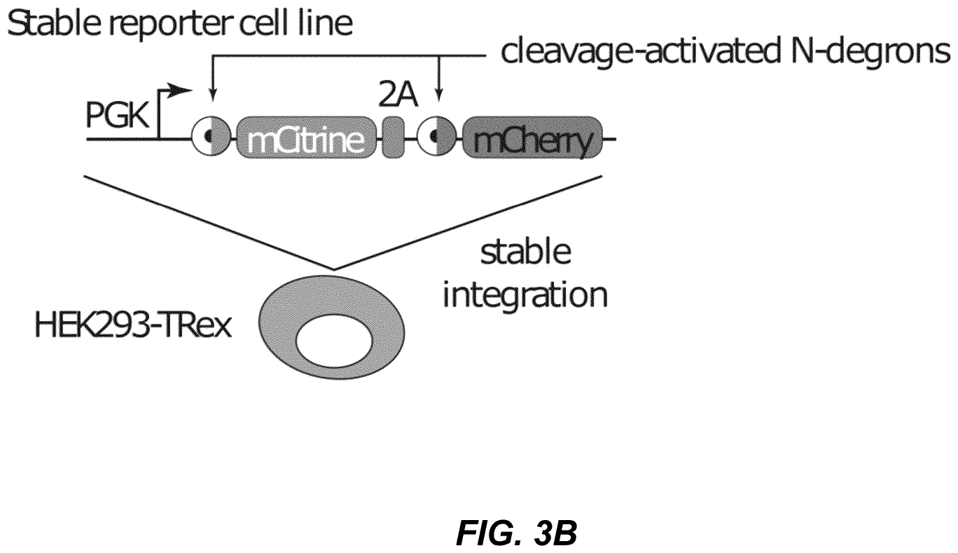

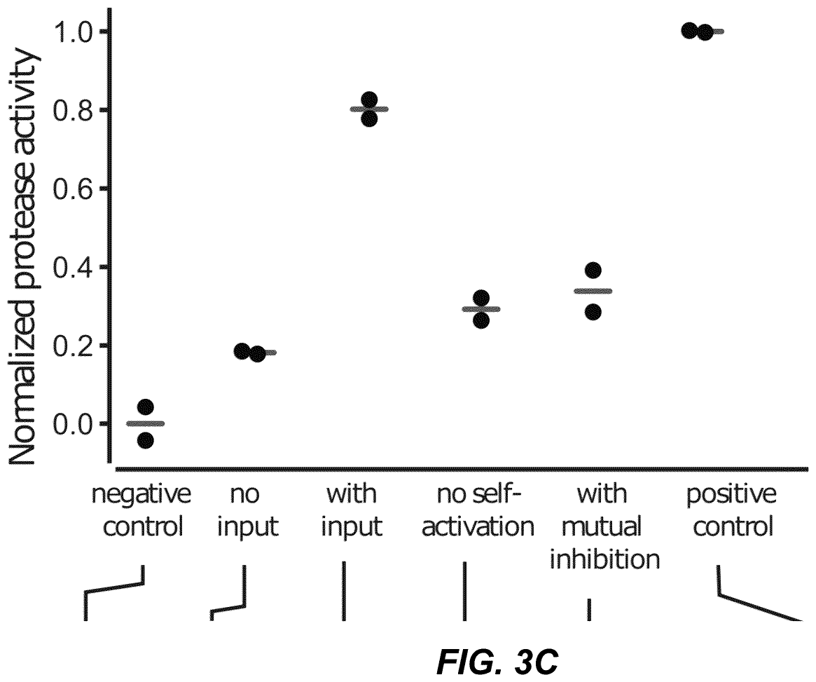

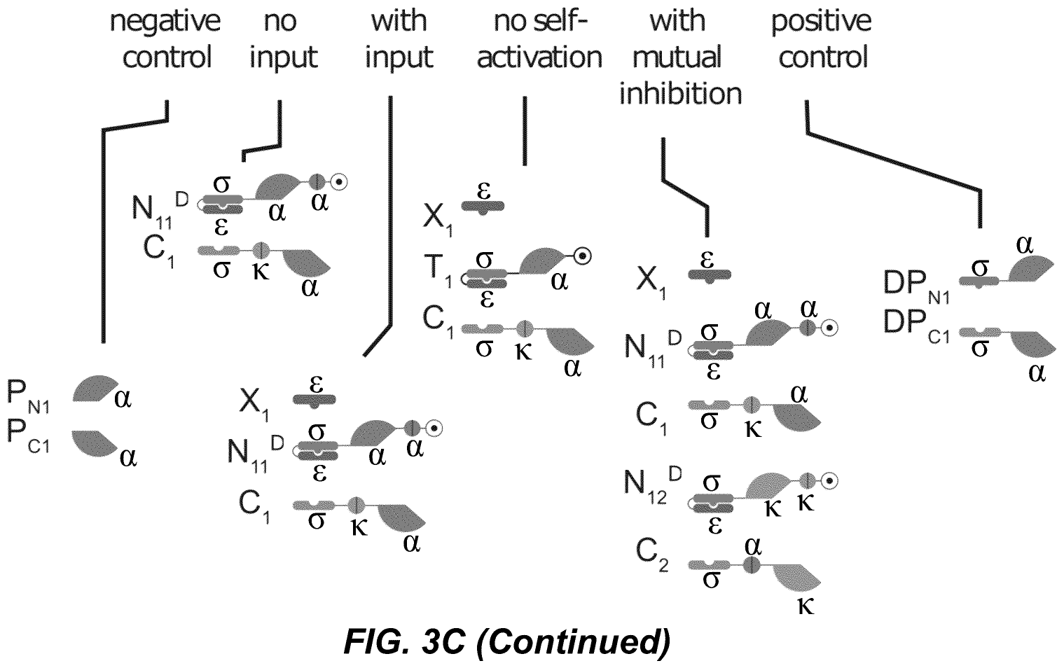

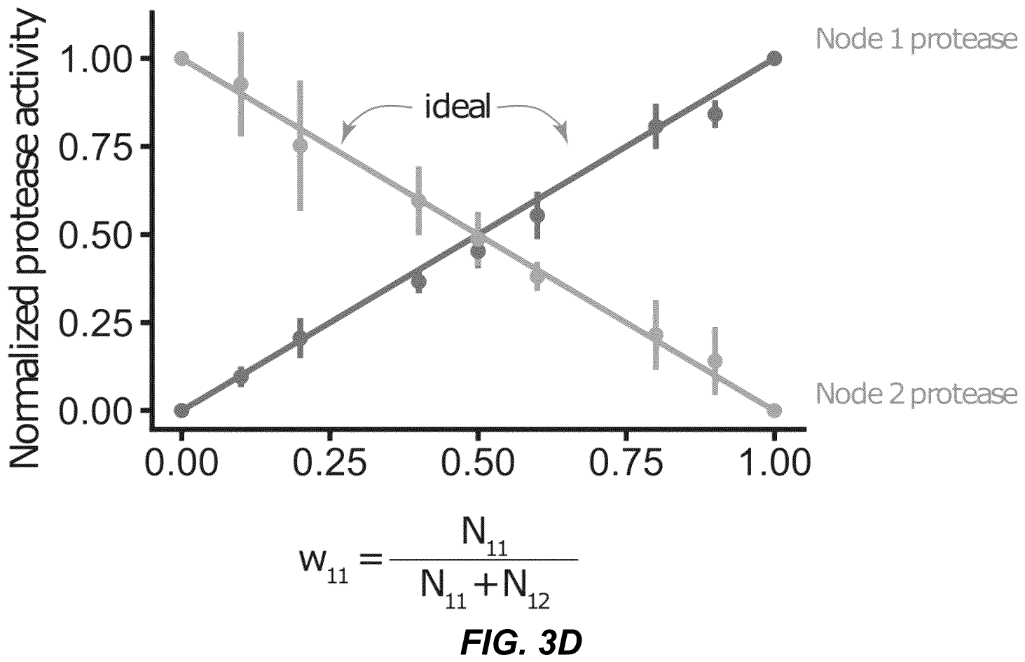

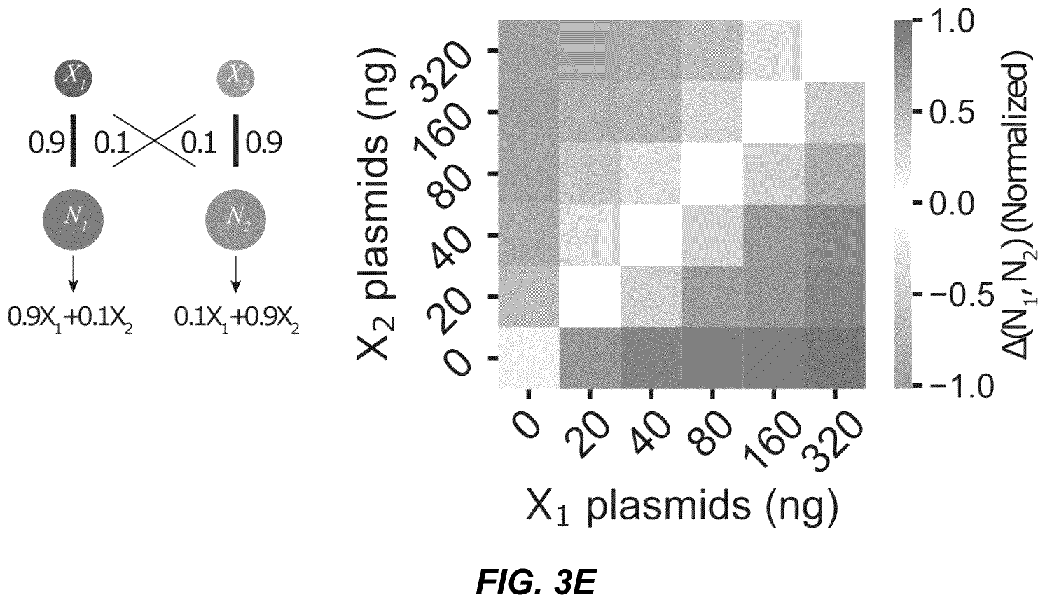

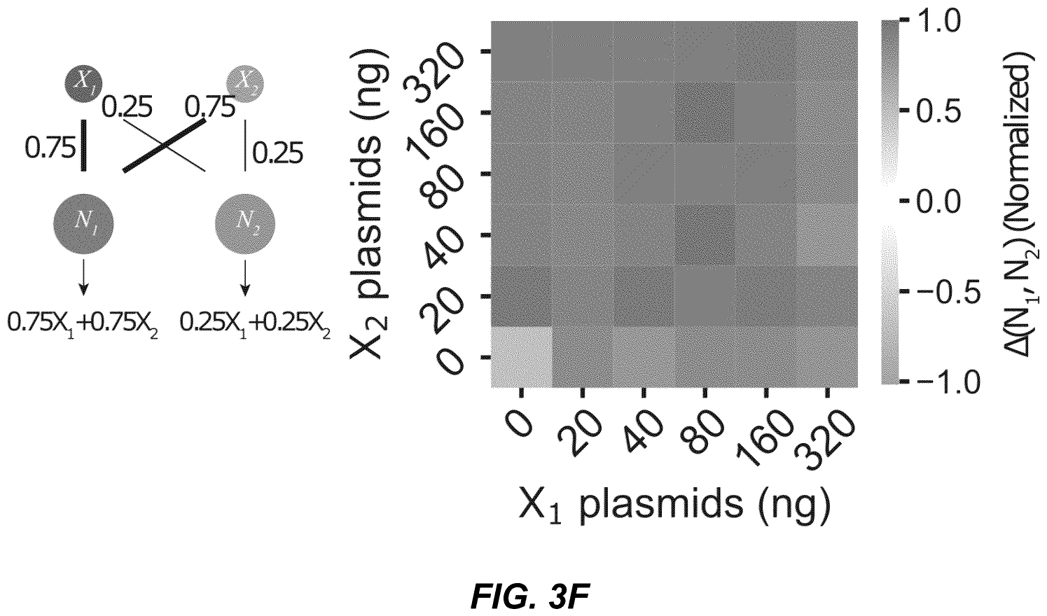

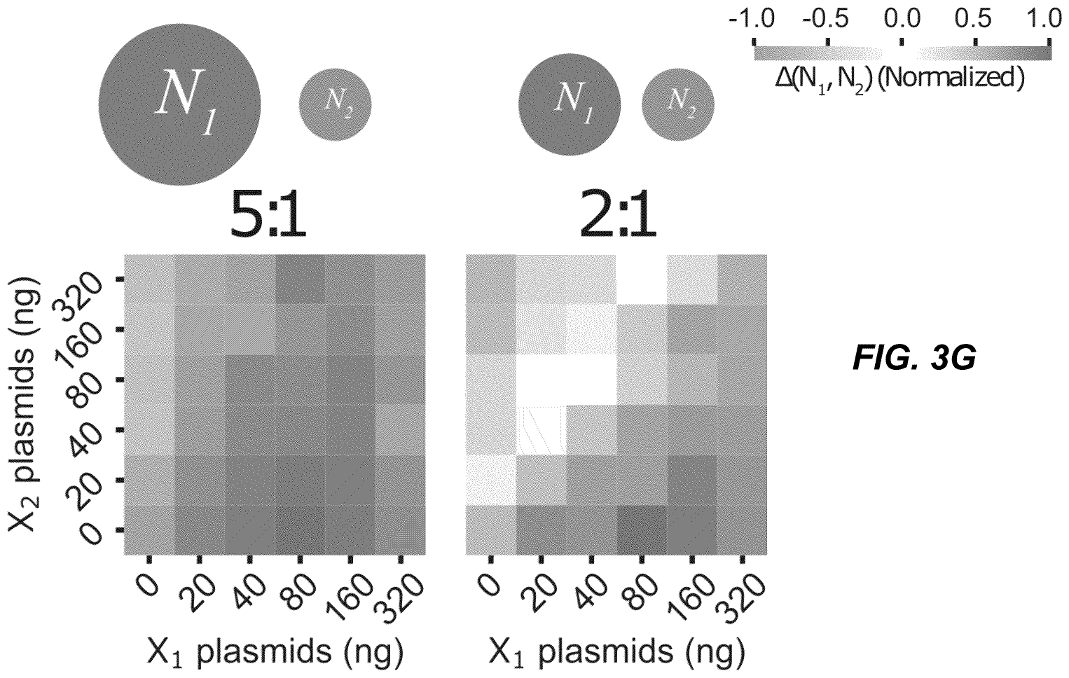

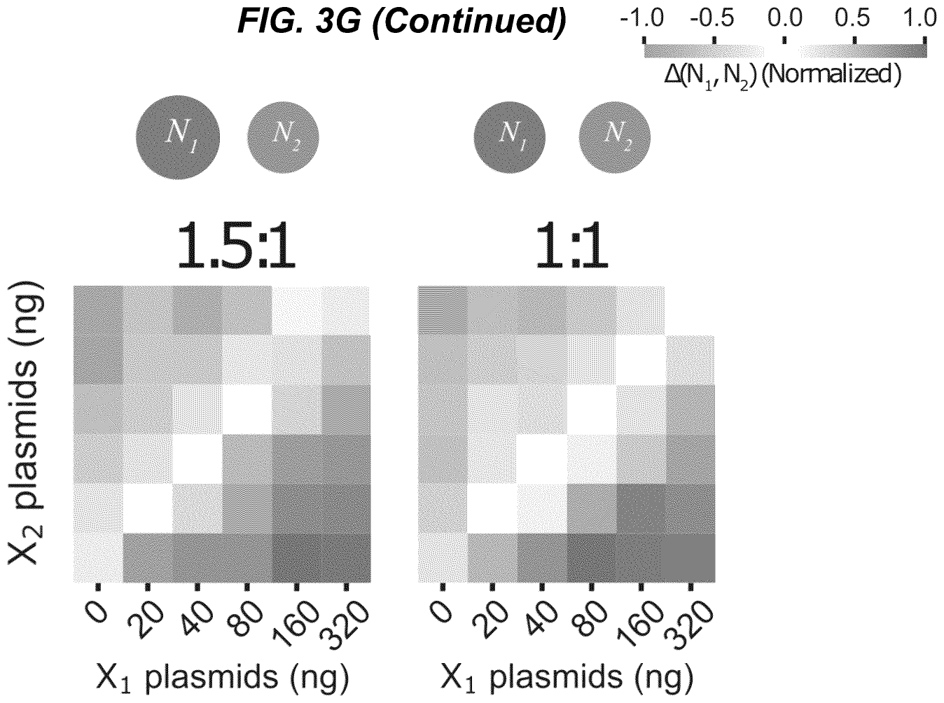

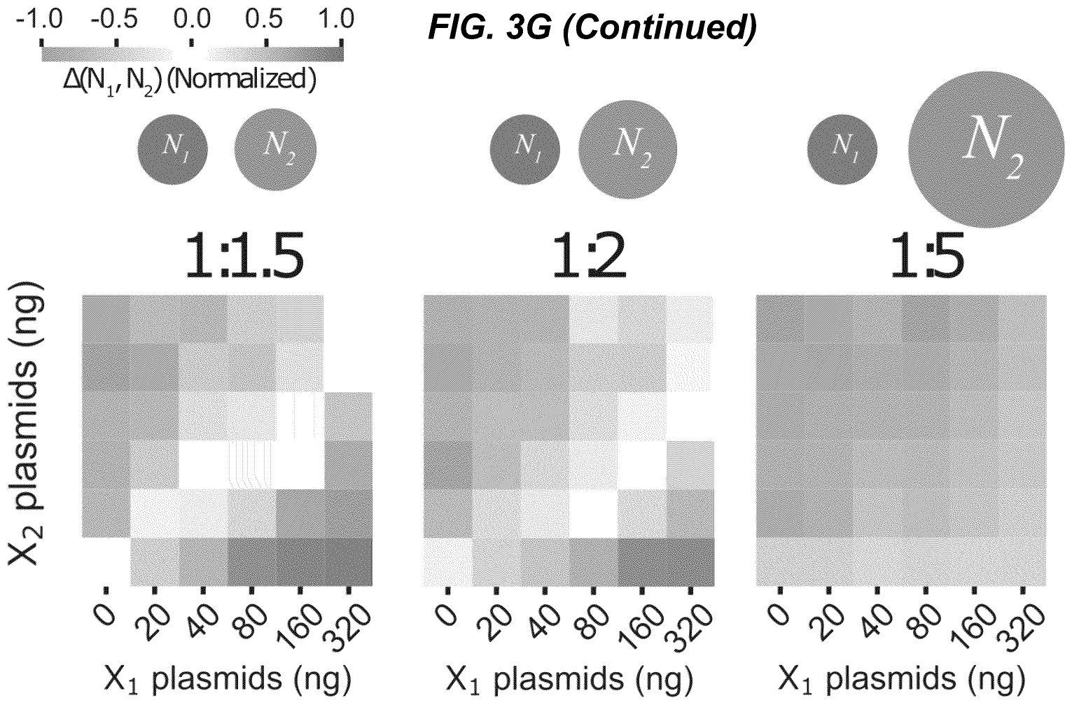

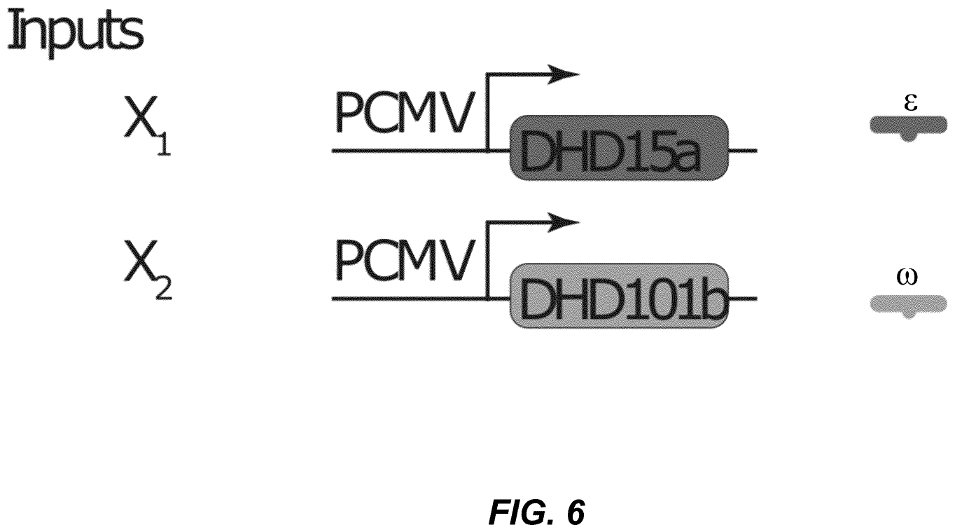

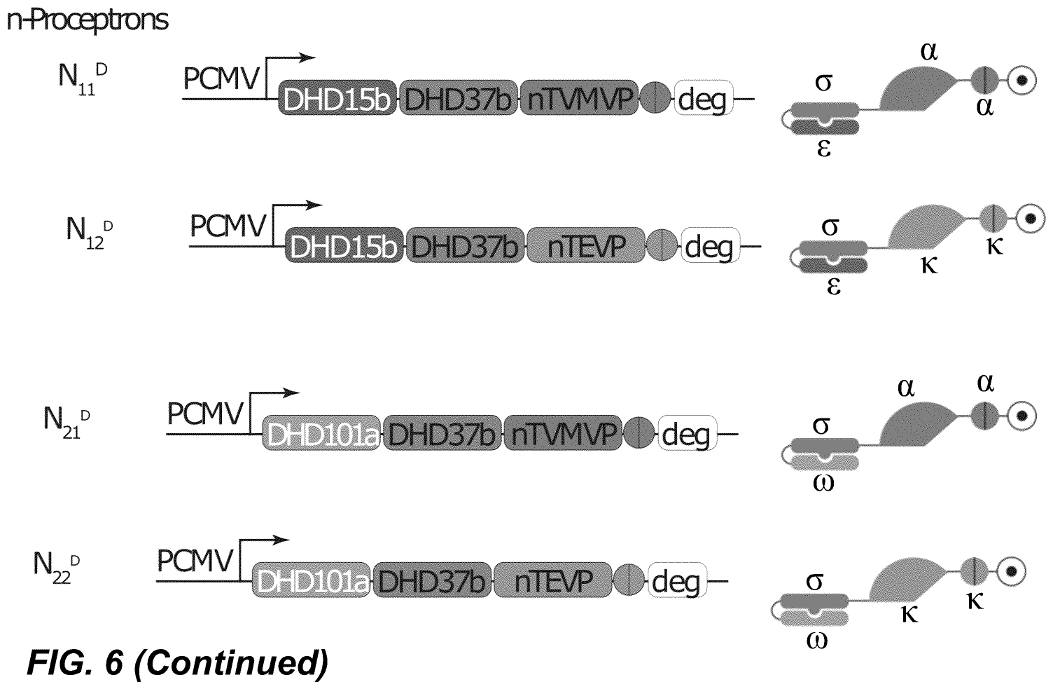

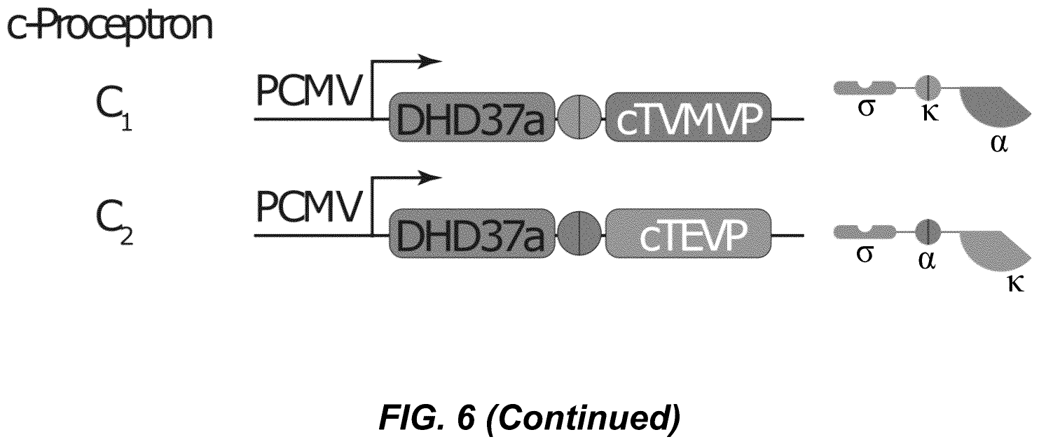

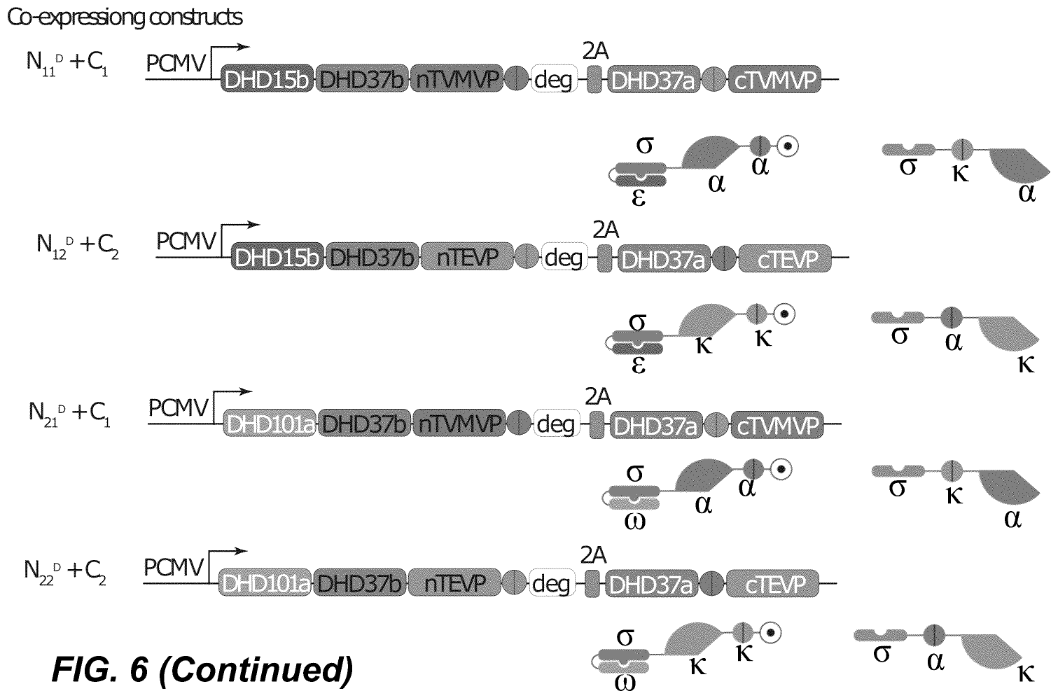

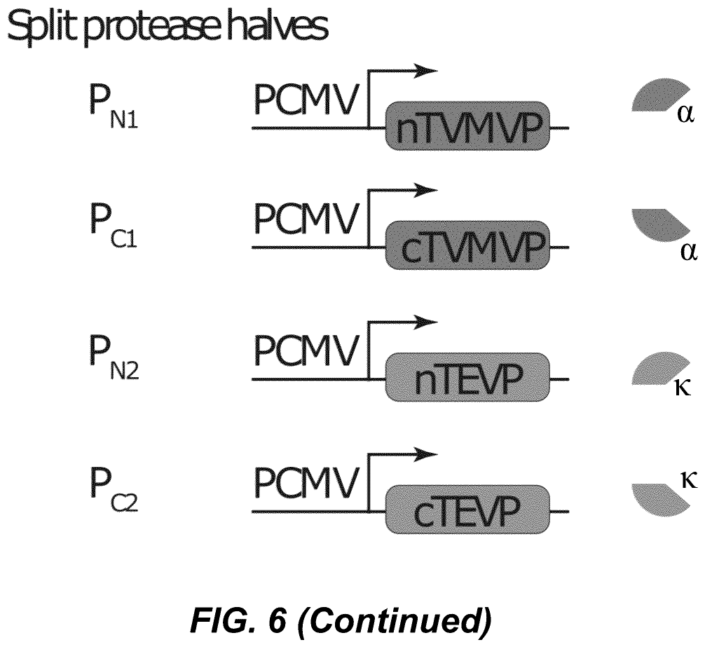

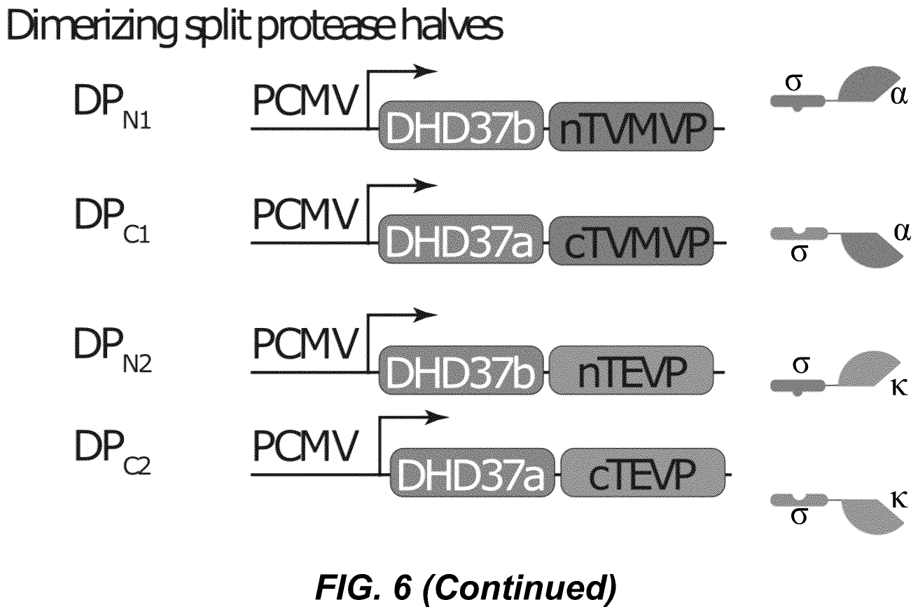

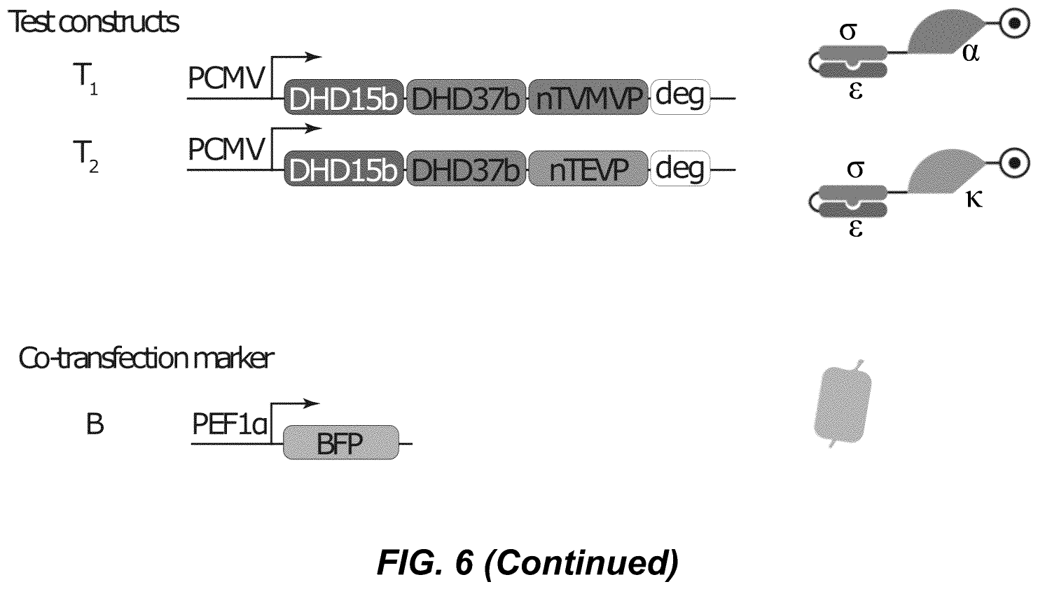

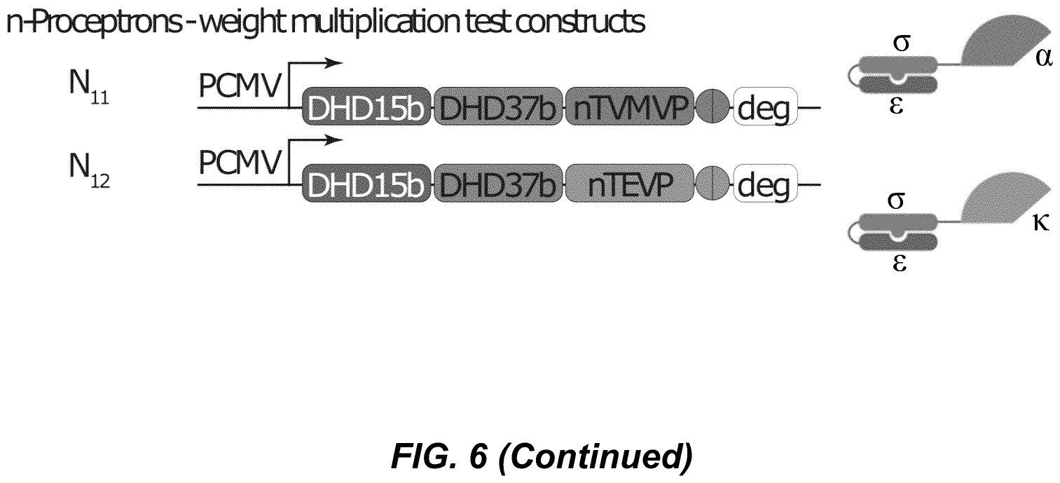

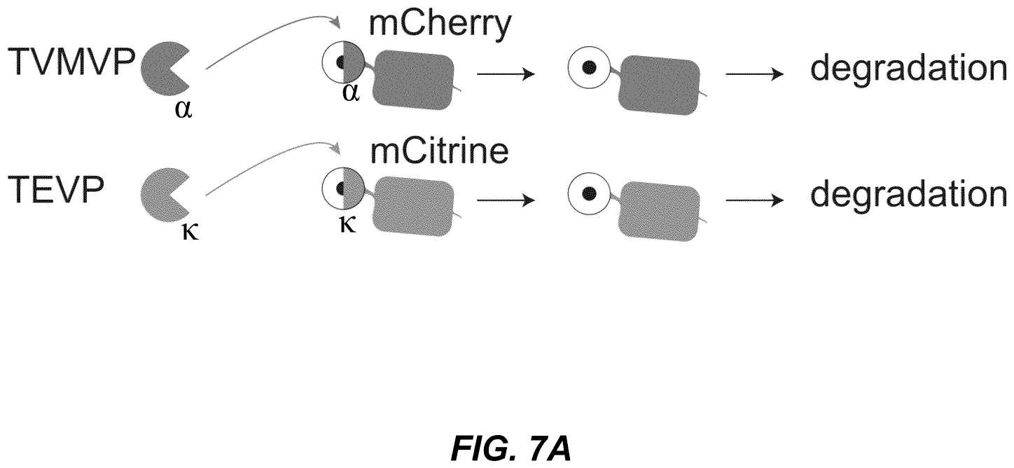

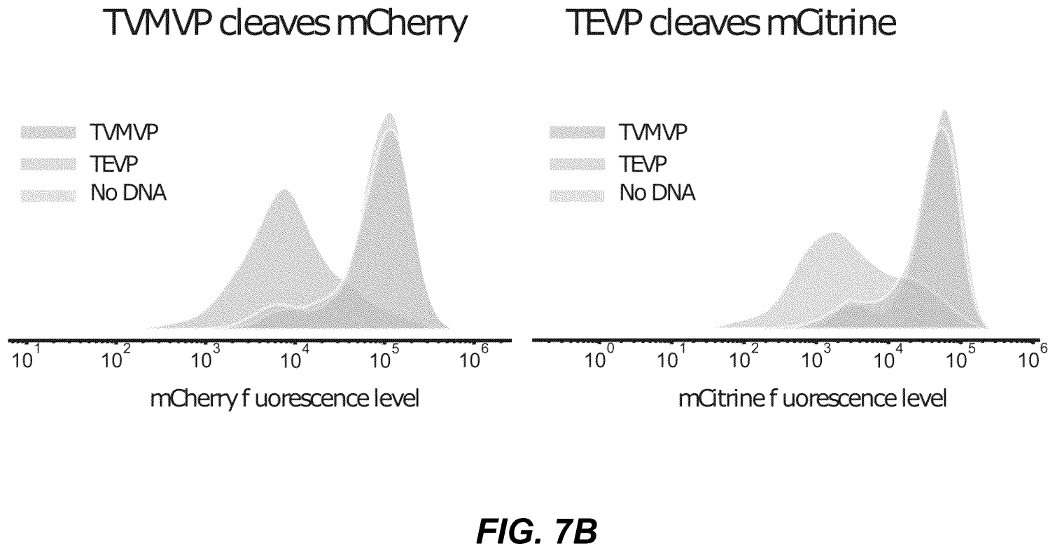

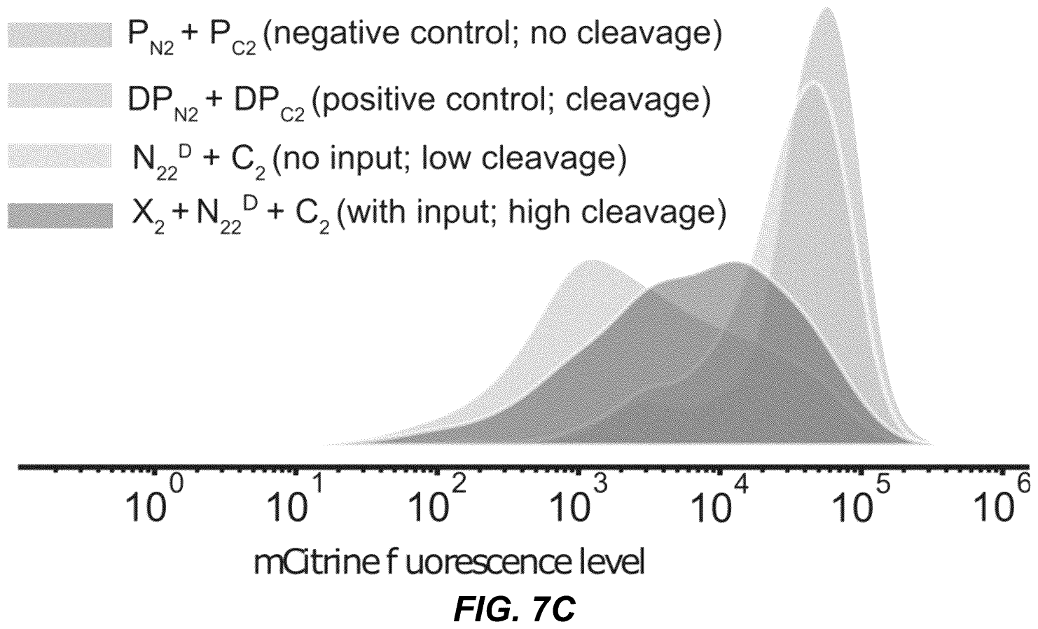

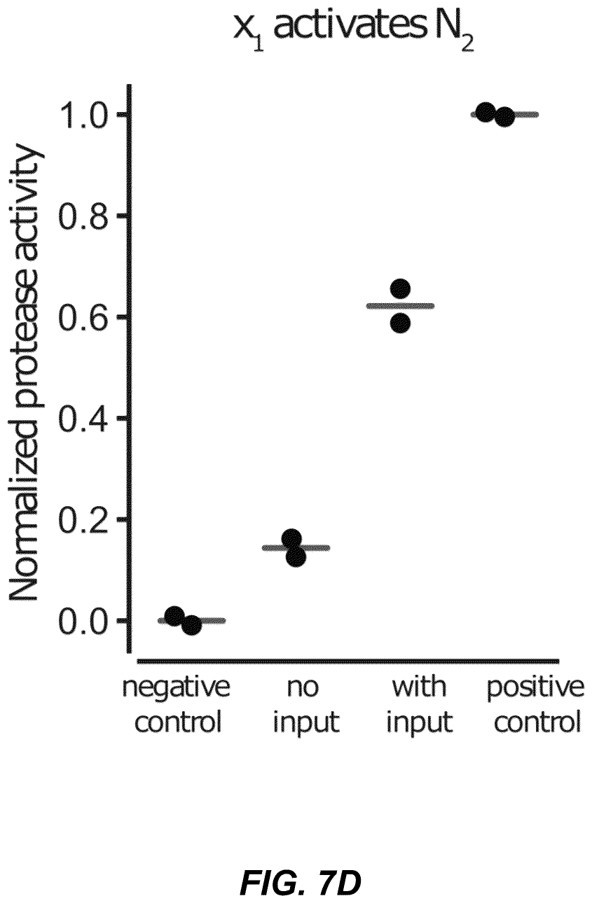

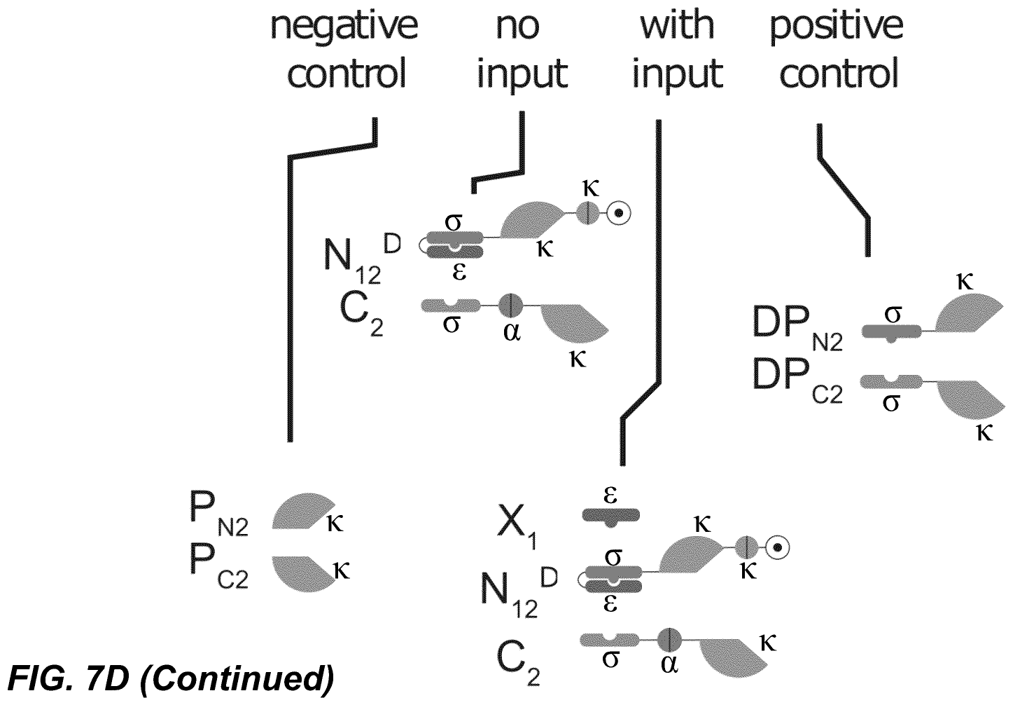

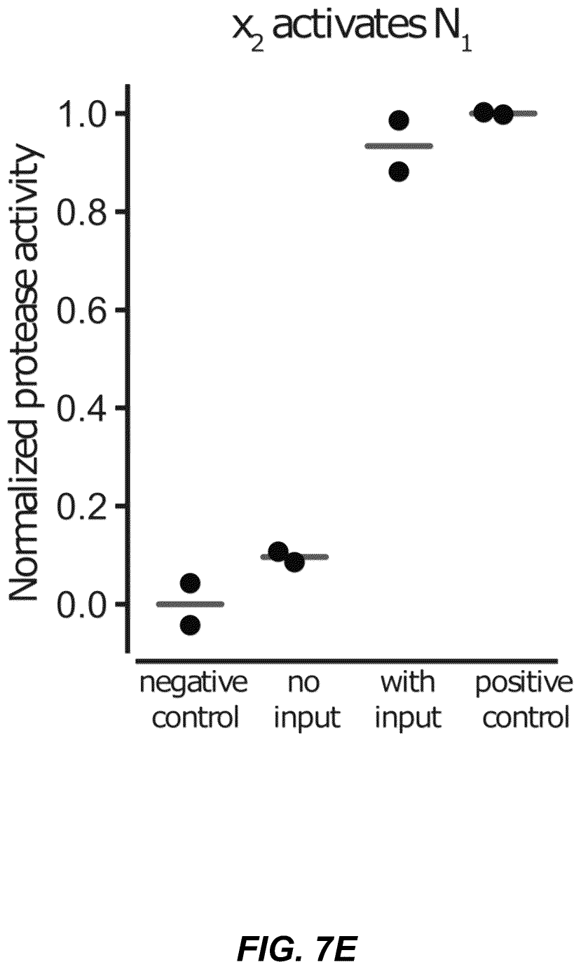

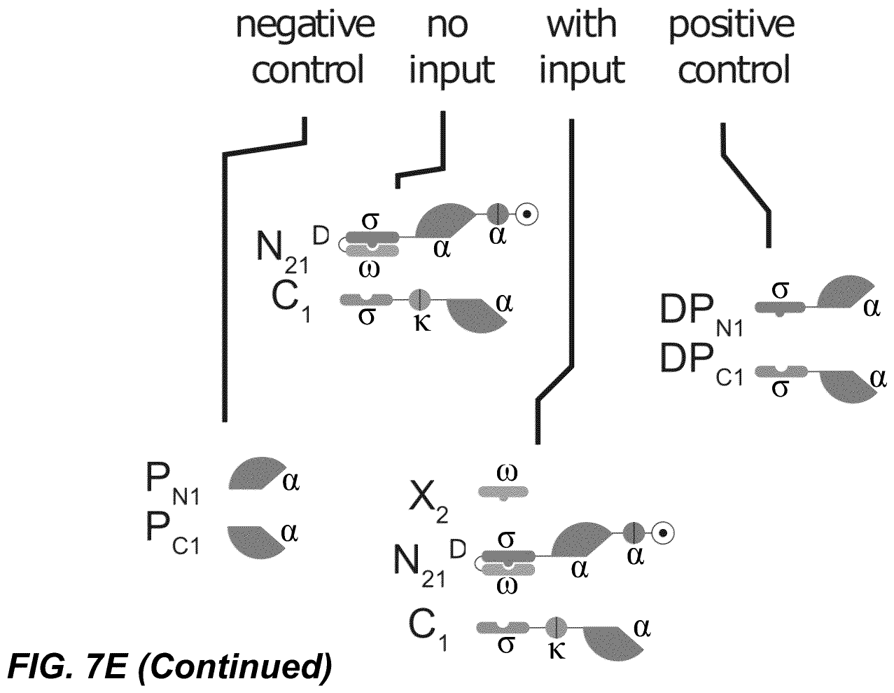

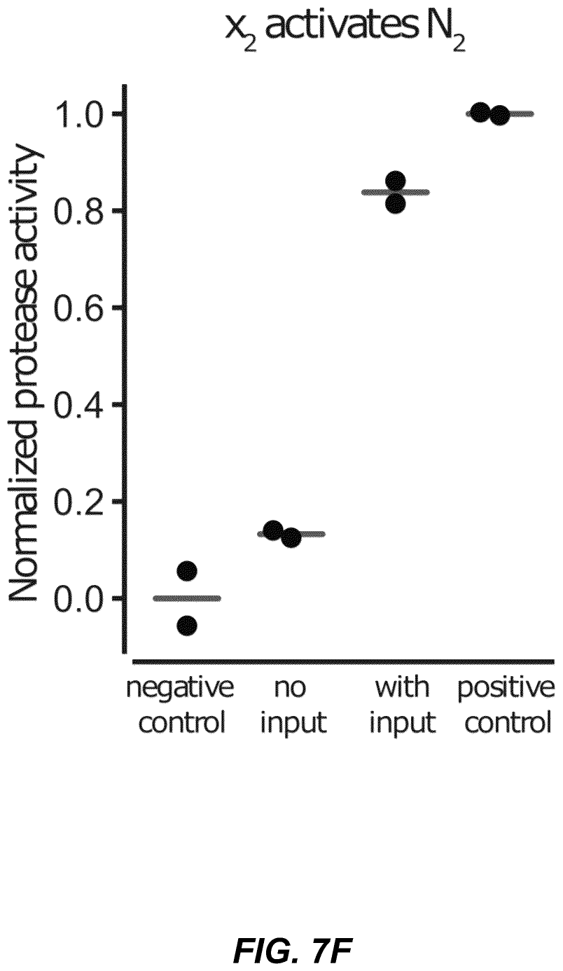

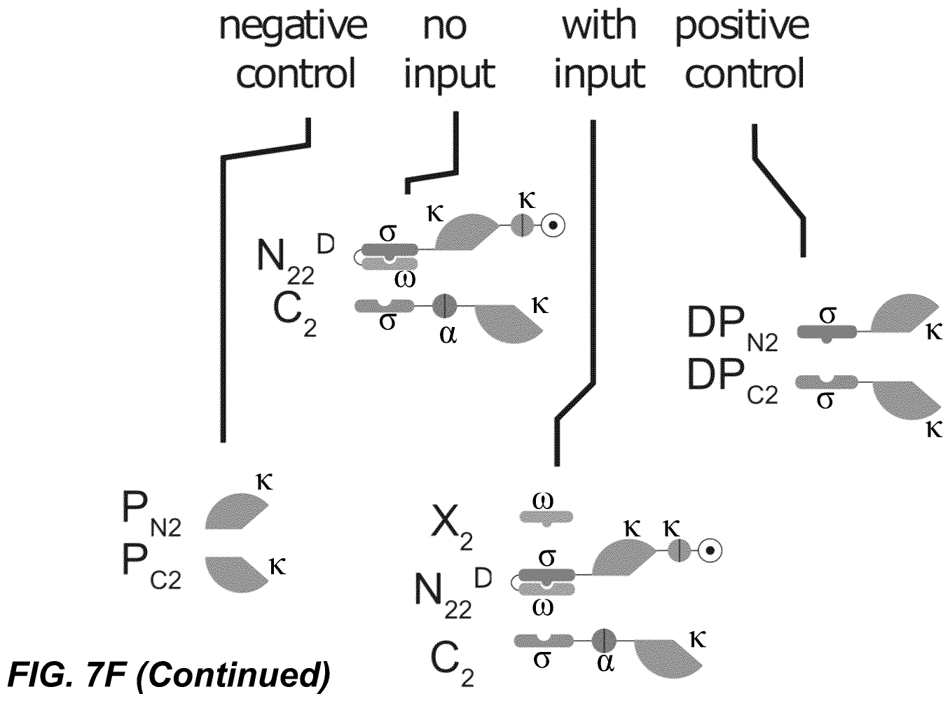

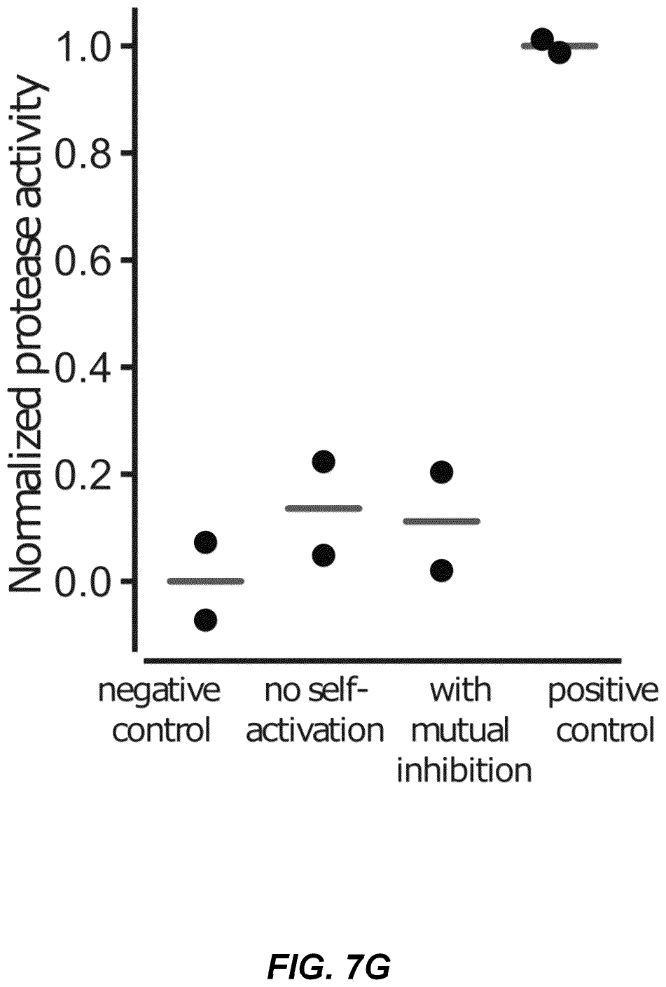

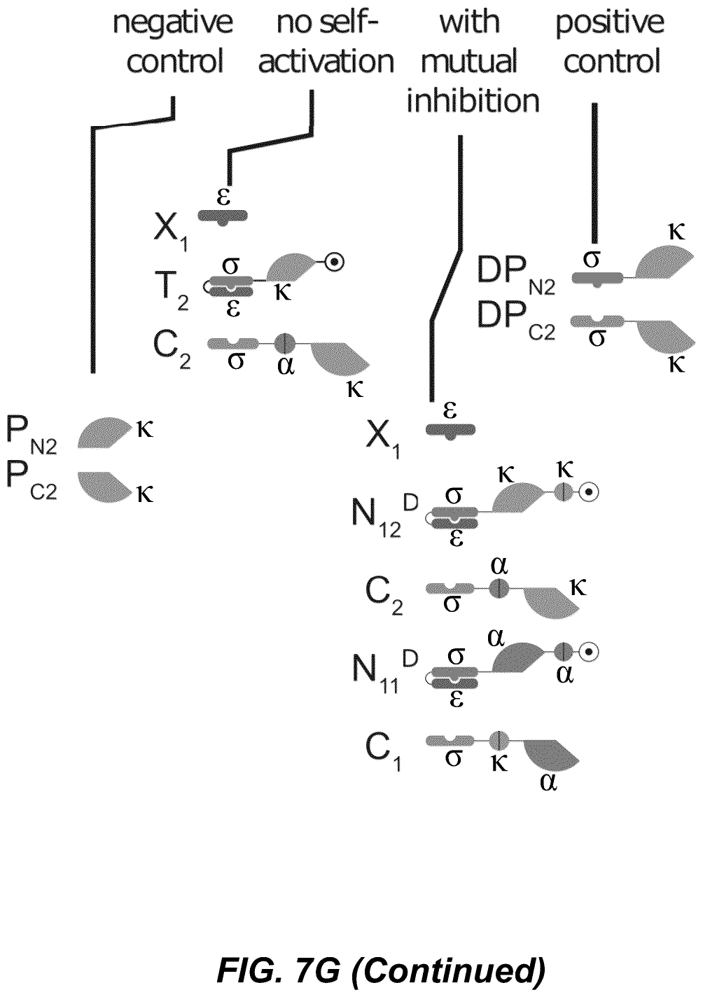

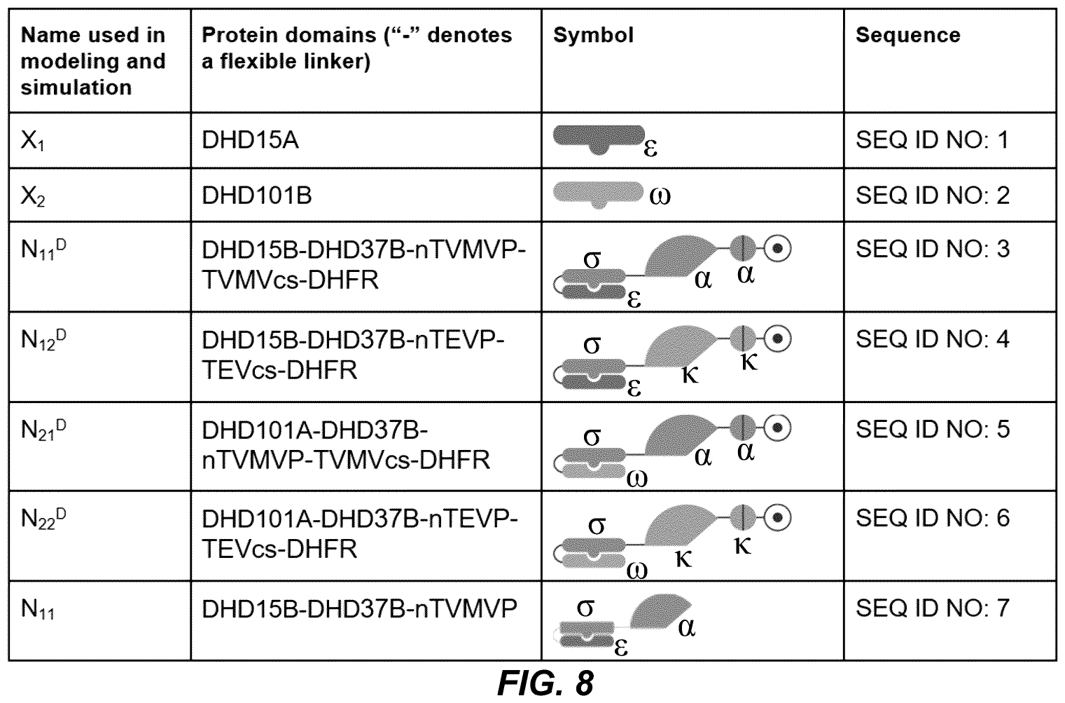

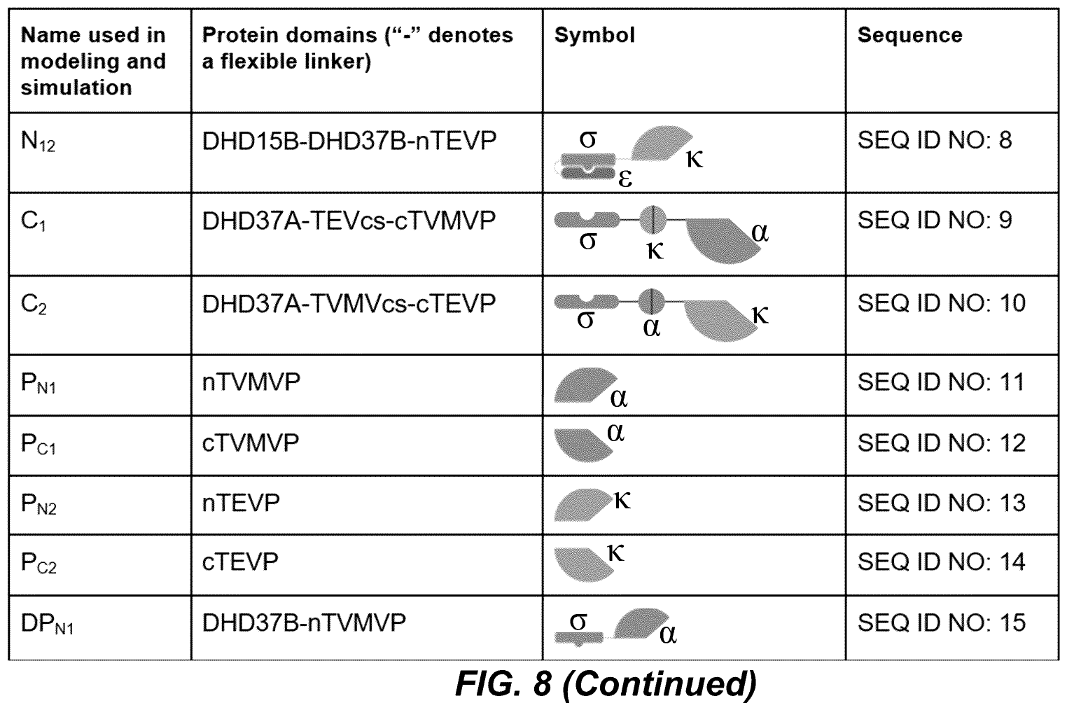

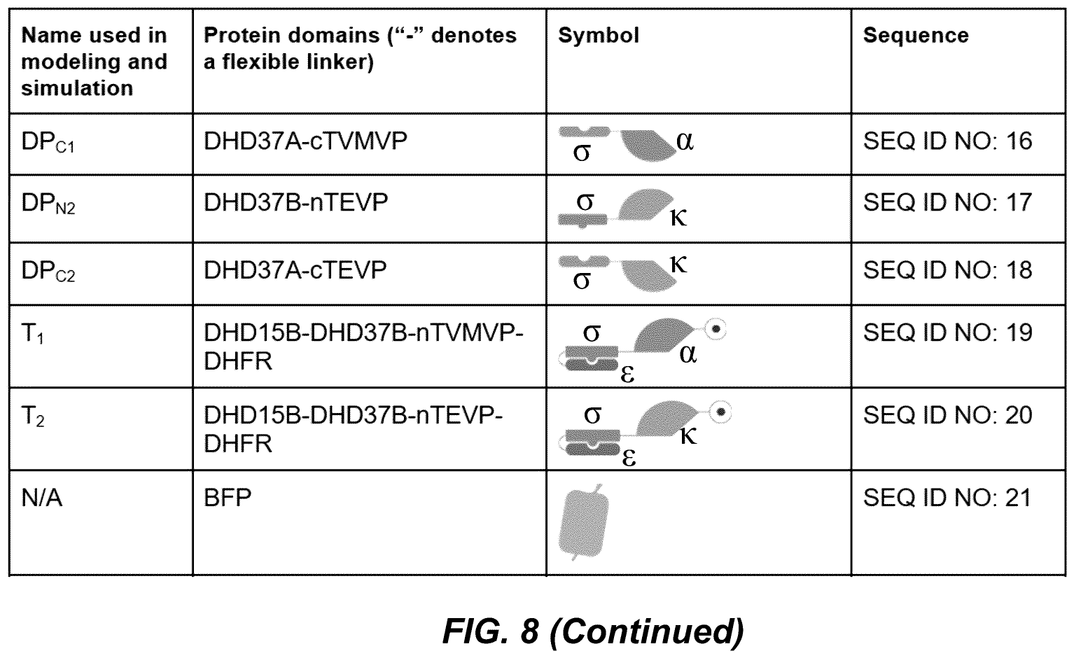

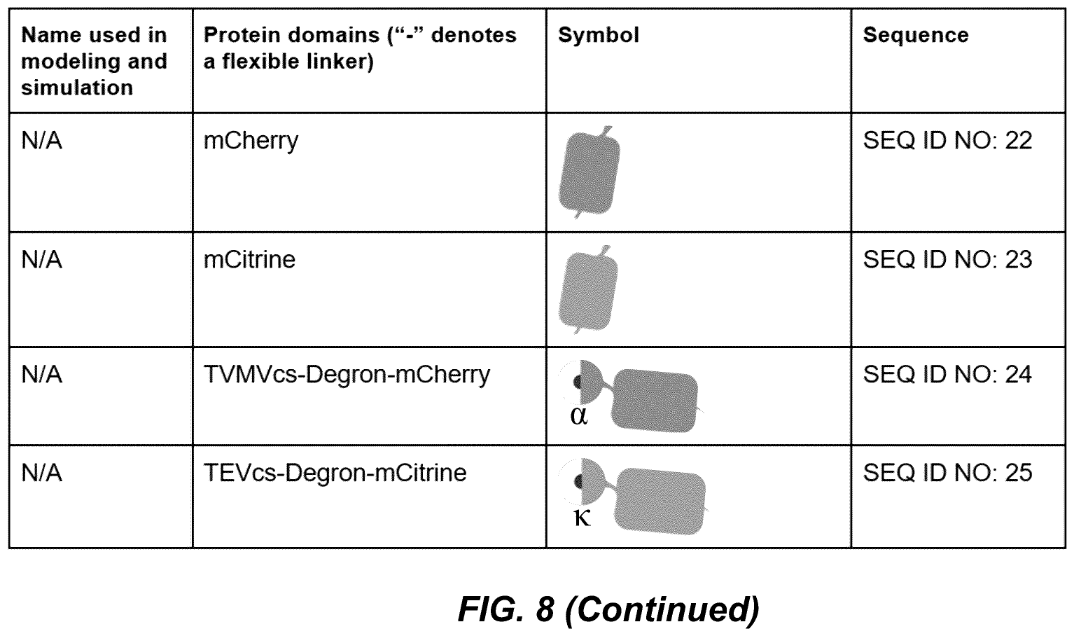

The set of 6 perceptein components and 2 input proteins necessary to implement the full circuit was constructed (FIG. 3A, FIG. 6). The split tobacco etch virus protease (TEVP) and tobacco vein mottling virus protease (TVMVP) were chosen as the two orthogonal proteases, and genetically fused the split halves to DHD domains, protease cleavage sequences, and degradation domains for controlled reconstitution of full proteases (FIG. 3A, FIG. 6, FIG. 8). In order to test many combinations of components and expression levels by co-transfection, each protein was encoded on a distinct plasmid. To measure the output of the circuit, a stable HEK293 reporter cell line was engineered containing a multi-cistronic construct co-expressing two fluorescent proteins—mCitrine and mCherry—each tagged with a cleavage-activated N-degron for either of the two input proteases (FIG. 3B). It was verified that each protease variant exclusively reduced fluorescence from its target reporter (FIG. 7A). Together, these constructs and the reporter cell line permitted rapid, iterative testing of circuit designs.

Using these components, each module of the winner-take-all neural network was experimentally validated, starting from the top of FIG. 1D. Each experiment was performed with varying amounts of input plasmids to identify combinations that maximize dynamic range and minimize background activities. First, it was asked whether inputs could trigger reconstitution of corresponding protease activities, as depicted in FIG. 1E. Co-transfecting input X₁ with cognate N- and C-node proteins inactivated the corresponding fluorescent protein reporter, consistent with reconstitution of the corresponding protease (FIG. 3C, FIG. 7C, FIGS. 7D-7F). Input-triggered protease activities were comparable to positive controls, consisting of split protease halves fused to heterodimerizing domains. In the absence of input, reporter levels were similar to those in a negative control consisting of split protease halves lacking DHD domains. These results suggest that inputs can reconstitute cognate protease activities.

Next, the weight multiplication step was focused on, which should ideally distribute input proteins based on the relative abundance of N-node proteins (FIG. 1G). In order to obtain a homogeneous distribution of constructs in each cell, mRNAs were used encoding test constructs for transfection. Cells were transfected with varying ratios of N₁₁ and N₁₂, while keeping X₁ constant. The amount of activated (protease reconstituted) N₁ and N₂, which determines the fluorescence of mCitrine and mCherry, should be linearly dependent on the ratio of N₁₁ and N₁₂ expression levels. Flow cytometry analysis confirmed that the changes in fluorescence followed a linear trend consistent with the concentration ratios of transfected N₁₁ and N₁₂ constructs (FIG. 3D).

Once activated, the perceptein components can self-activate and mutually inhibit (FIG. 1F). Self-activation involves protease cleavage-dependent removal of a fused DHFR degron from the half protease, stabilizing proteases of its own kind. To evaluate the extent of self-activation, HEK293 cells were transfected with plasmids encoding either N-node constructs with a protease cleavable degron, or similar negative control constructs lacking the cleavage site that are therefore unable to self activate. Flow cytometry revealed a 5 fold change in protease activity upon self-activation. By contrast, the negative control remained close to the background (FIG. 3C and FIG. 7G). Mutual inhibition interactions (FIG. 1F) also functioned as expected. As shown in FIG. 3C, protease activities of the N₁ node were strongly repressed by an excess of the N₂ node components (N₁₂^{D} and C₂). Mutual inhibition worked similarly in the opposite direction when there was more N₁ than N₂ (FIG. 7G). Taken together, these results indicate that each module of the full circuit can function individually.

To test whether the full circuit behaved as predicted, reporter cells were co-transfected (FIG. 3B) with varying concentrations of the inputs, fixed amounts of the N- and C-half-node proteins (in a multicistronic manner), and a BFP co-transfection marker (FIG. 6), and read out reporter fluorescence. The concentrations of the N₁₁^{D} and N₂₂^{D} plasmids were set 9 times higher than the concentrations of the N₁₂^{D} and N₂₁^{D} plasmids to put the circuit into a comparative regime (FIG. 3E, left). Protease activities were normalized based on fluorescence (Materials and methods), and plotted the differences in normalized protease activities in N₁ and N₂. As expected, the output was positive when X₁ exceeded X₂ and negative when X₂ exceeded X₁, with minimal response at equal input concentrations (FIG. 3E, right). The output became more binary with greater absolute difference between the two inputs, approaching the binary response observed in simulations. Additionally, varying the relative levels of perceptein components resulted in a biased comparator where, in agreement with prediction (FIG. 3F, left), Node 1 was the winner regardless of the two input values (FIG. 3F, right). These results show that the full circuit can compare the relative levels of two inputs.

In the model, eliminating the cross interactions, X₁−N₂ and X₂−N₁ altogether, leads to a simpler comparator regime that should allow input classification with a decision boundary whose position can be tuned by modulating the relative expression levels of the perceptein components (FIG. 2A, second row). To test this capability, cells were transfected with varying relative levels of the N₁₁^{D} and N₂₂^{D} plasmids, while omitting the N₁₂^{D} and N₂₁^{D} plasmids entirely. At a 1:1 ratio of N₁₁^{D}:N₂₂^{D}, similar classification behavior was observed to that seen with the full circuit (FIG. 3E). Modulating the relative levels of N₁₁^{D} and N₂₂^{D} shifted the decision boundary, similar to predictions (FIG. 3G). These results support the ability of the circuit to enable tunable classification.

The Perceptein can Produce Scalable Classification

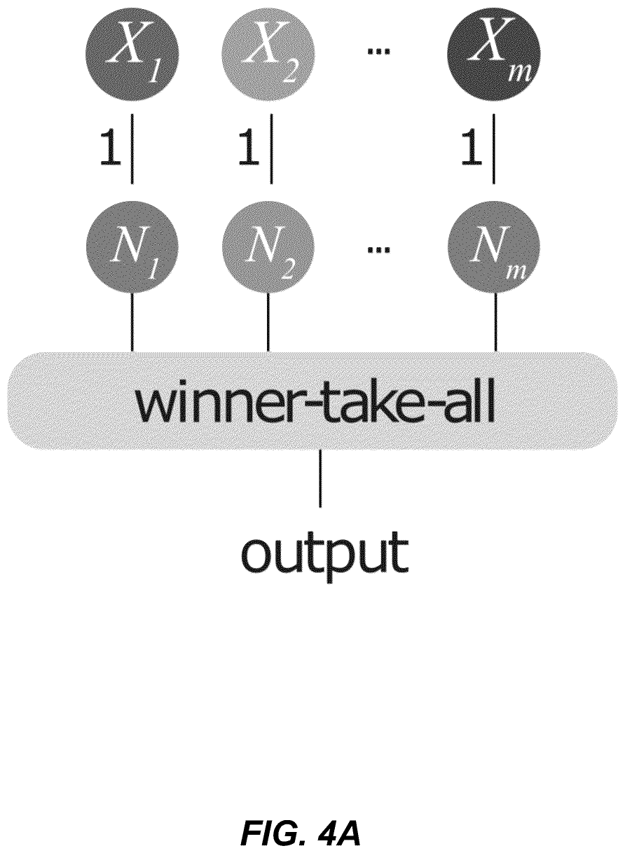

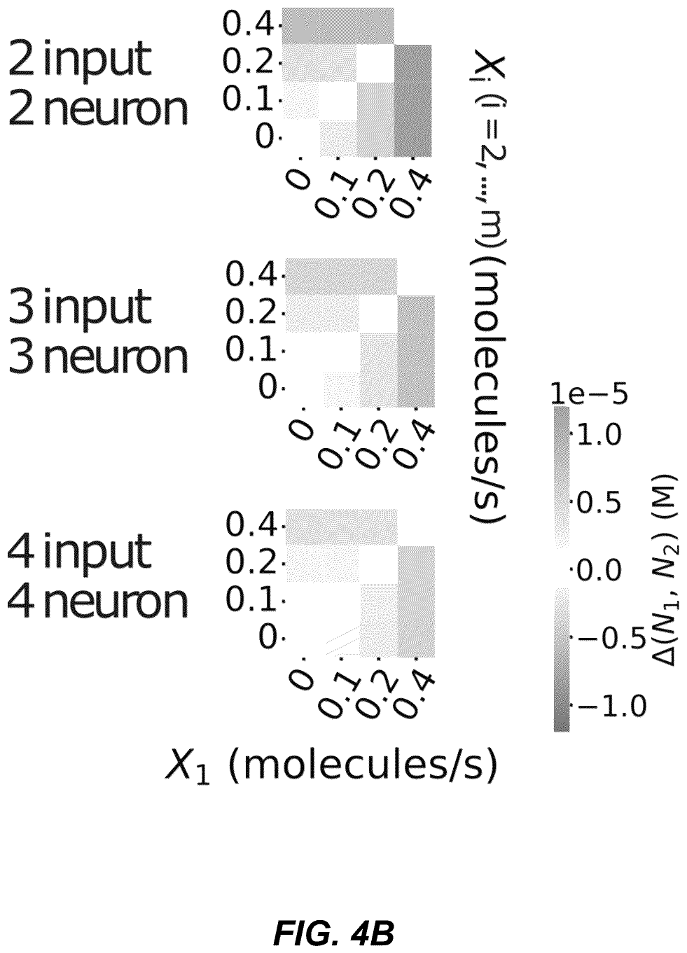

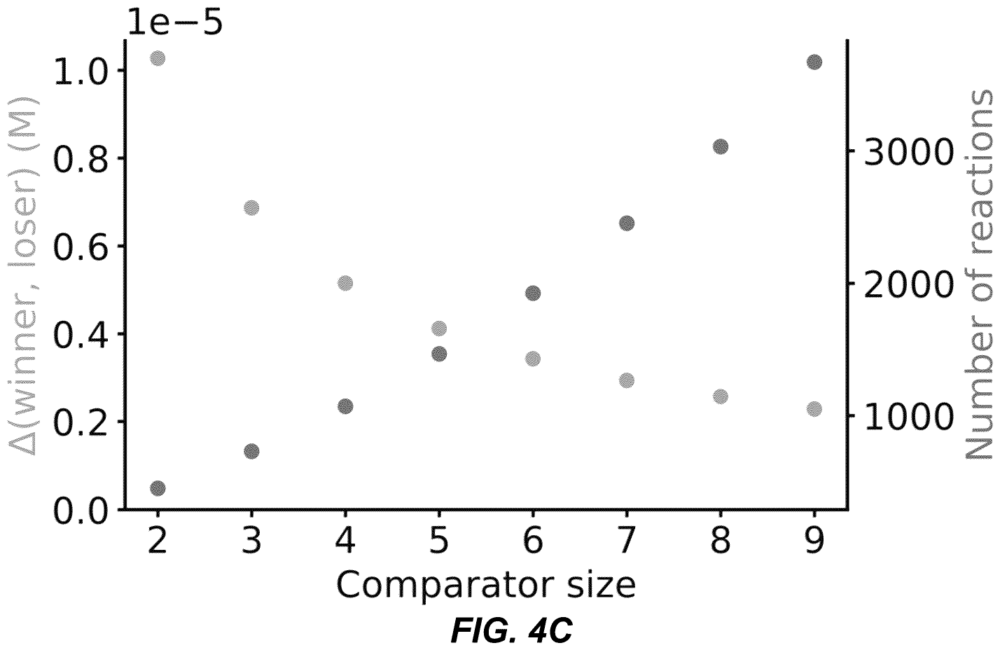

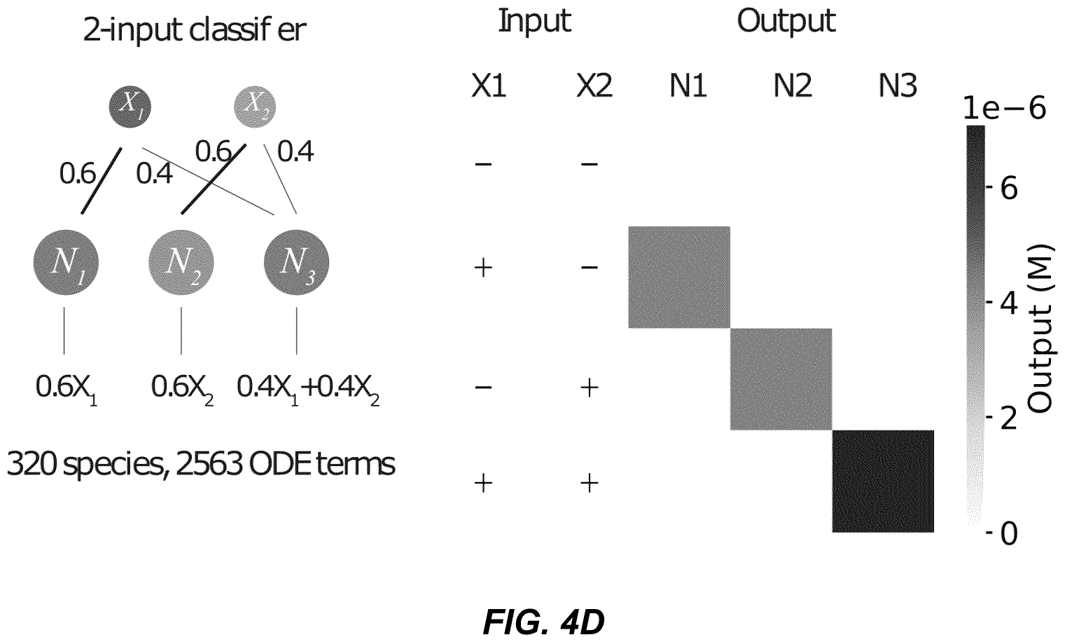

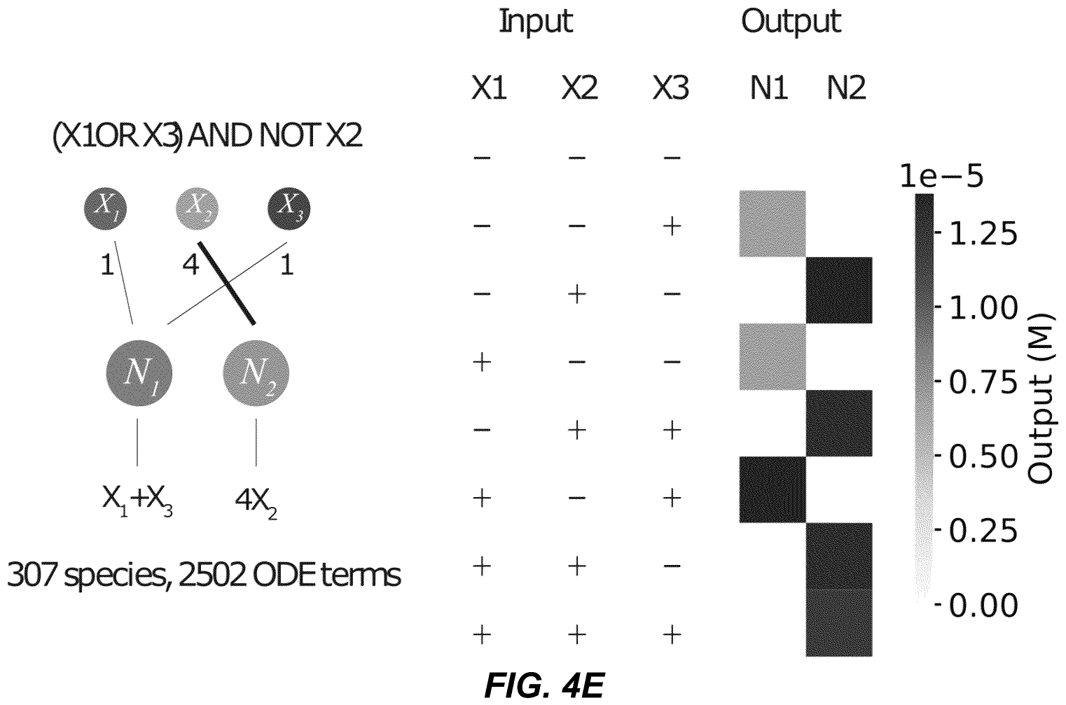

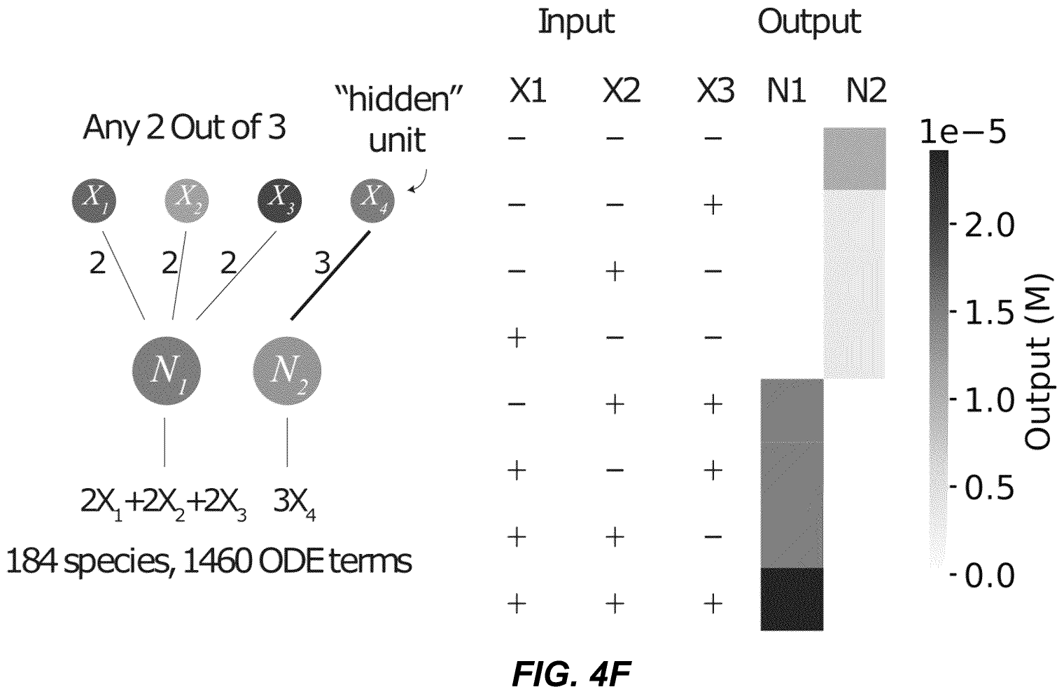

How well can this protein-based neural network scale? To address this question, higher dimension comparators were simulated, each composed of m inputs and m nodes (FIG. 4A). For each value of m, the response to a matrix of input values was simulated. Larger systems retained classification ability, despite some loss of output dynamic range (FIGS. 4B-4C). Overall circuit complexity, measured by the number of chemical reactions, scaled approximately linearly with the size of the comparator, m (FIG. 4C).

Finally, simulations showed that the perceptein system can also perform more complex types of classification. For example, by adding a third node to the two-input classifier, one can obtain a winner-take-all response in which nodes 1 and 2 respond to X₁ or X₂ alone, while node 3 responds only to the presence of both (FIG. 4D). Conversely, with three inputs and two nodes one can, in a single layer system, compute composite functions such as (X₁ OR X₃) AND NOT X₂ that require multiple layers of conventional Boolean logic gates (FIG. 4E). One can also augment the system with “hidden” units that establish thresholds for the true inputs in order to compute even more complex functions. For example, by adding a single hidden unit to the 3-input, 2-node system, one can obtain a circuit that computes an ANY 2 OUT OF 3 function, where node 1 is activated when at least two of the three inputs are present (FIG. 4F). Together, these results show that the perceptein system can in principle be scaled and extended to solve a broader variety of classification problems.

### Discussion

Here, inspired by the classic winner-take-all neural network design, the perceptein architecture for protein level winner-take-all classification was introduced within living mammalian cells. The circuit design is based on three design principles: First, three-way cooperative binding interactions enable input-dependent protease activation, producing the species on the right in FIG. 1E. These cooperative interactions are in turn enabled by the use of fusions of de novo designed heterodimers. Analogous functions could in principle be achieved with naturally cooperative protein binding systems such as those in the N-WASP system. However, the de novo designed proteins offer a larger repertoire of distinct binding specificities, and minimize unintended interactions with endogenous cellular proteins. Second, the design takes advantage of the irreversibility of protease-based cleavage, in combination with degrons. Self-activation involves proteolytic degron removal, while mutual inhibition is achieved by proteolytic inactivation (FIG. 1F). In some embodiments provided herein, other mechanisms, such as post translational modification, can also be exploited to allow orthogonal signal processing and enable multi-layer protein networks. In some embodiments of the synthetic proteins circuits provided herein, said mechanisms include, but are not limited to, one or more of the following: phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, and cleavage. Thus, for example, in some embodiments of the circuits provided herein, the first part of a first protease domain is swapped with the first part of a first kinase domain, the second part of a first protease domain is swapped with second part of a first kinase domain, a first part of a second protease domain is swapped with a first part of a second kinase domain, and a second part of a second protease domain is swapped with a second part of a second kinase domain. Third, perceptein components further exploit molecular competition between protease halves. Splitting proteases and letting them compete to form productive or inactive complexes achieves two functional goals: it makes each protease activity dependent on a cognate input, and it supports the winner-take-all behavior, as protease halves effectively quench the activity of their non-matching partners (FIG. 1E). Combining these principles successfully enabled two-input winner-take-all classification in mammalian cells (FIG. 3).

A key feature of neural computation is the ability to modulate input-output functions by tuning weights in an existing network. Analogously, varying the expression levels of perceptein circuit components such as N₁₁^{D} and N₁₂^{D} systematically shifted the decision boundary in the classification circuit without requiring additional protein components (FIG. 2B, FIG. 3G). Because tuning can be achieved through expression, this design allows cells to control their own decision thresholds by modulating gene expression.

The perceptein architecture can be scaled up to perform higher dimensional classification tasks. A fully connected network (FIG. 2A, first row) with p inputs and q neurons grows multiplicatively, requiring a total of p+q+pq distinct proteins. However, in the case of the simpler comparator architecture (FIG. 2A, second row), elimination of cross interactions between inputs and node proteins leads to linear scaling, with p+q starting components. When considering scaling, it is interesting to also consider the remarkably large number of different protein species and complexes (e.g. 158 for the 2-input, 2-output system) that are generated in the operation of this system. Effectively, this large number of molecular species is “compressed” into the much smaller number (e.g. 8 for the 2-input, 2-output system) of starting protein species from which they are generated. This is reminiscent of the way certain protein families can produce huge diversities of protein products through alternative splicing, proteolytic cleavage of pro-proteins, or combinatorial assembly of alternative multimeric complexes. Previous mammalian synthetic biology work has used as many as 12 unique genes in a single system. To scale up protein computation further will require expression of more starting protein species.

Finally, the perceptein output proteins are proteases, whose activities can be engineered to control diverse targets, including activation of endogenous pathways, such as cell death; transcription factors; or other synthetic protein systems. They can also be wired to additional perceptein layers, allowing the construction of more powerful, and more fault-tolerant, multi-layer networks. Iterations of the designs provided herein can therefore enable the programming of more complex biochemical computations, rivaling those produced by evolution, in living cells.

### Materials and Methods

Construction of Synthetic Genes

Some constructs were generated using standard cloning procedures. The inserts were generated using PCR or gBlock synthesis (IDT), and were annealed by Gibson assembly with backbones that are linearized using restriction digestion. The rest of the constructs were designed by authors and synthesized by Genscript. mRNAs were ordered from TriLink BioTechnologies. A list of all constructs used in this study is included in FIG. 8.

Tissue Culture

The monoclonal reporter cell line HEK1012 was generated using the PiggyBac Transposon system (Systems Biosciences) in Flp-In™ T-REx™ Human Embryonic Kidney 293 cell line (HEK293-TRex, Thermo). The plasmid with 3-phosphoglycerate kinase (PGK) promoter driving the expression of mCitrine and mCherry with N-end protease activatable degrons in PiggyBac backbone was co-transfected with a Super PiggyBac Transposase plasmid into HEK293-TRex cells. 24 hours after transfection, cells were transferred into a 6-well plate and selected with 400 μg/ml Zeocin for 9 days (split into Zeocin media every 3 days). The resulting polyclonal cells were then diluted at 1 cell/well into 96-well plates. After a week, wells with a single clone and positive mCitrine and mCherry fluorescence were labeled. The HEK1012 line was one of the clones that has medium mCitrine and mCherry expression from flow cytometry measurement. Cells were maintained in Eppendorf CellXpert cell culture incubators at 37° C. with 5% CO₂. Cells were grown in media containing Dulbecco's Modified Eagle Medium (Gibco) supplemented with 10% Fetal Bovine Serum (Avantor), 1 mM sodium pyruvate (Gibco), 10 unit/ml penicillin (Gibco), 10 μg/ml streptomycin (Gibco), 2 mM L-glutamine (Gibco) and 0.1 mM MEM non-essential amino acids (Gibco).

Transient Transfection of DNA into Reporter Cells

HEK1012 reporter cells were seeded in 24-well plates at a density of 0.05-0.1×10⁶ cells per well and cultured for 24 hours. Transient transfection was performed the following day using Lipofectamine 2000 (Thermo Fisher) following the manufacturer's protocol. 100 ng/mL doxycycline was added to the growth media whenever expression is needed from a CMV-TO promoter. Cells were changed into fresh growth media, with 100 ng/mL doxycycline, 24 hours after transfection, and were analyzed by flow cytometry after 24 hours.

Transient Transfection of mRNA into Reporter Cells

HEK1012 reporter cells were seeded in 24-well plates at a density of 0.05×10⁶ cells per well and cultured for 24 hours. Transient transfection was performed the following day using the TransIT®-mRNA Transfection Kit (Minis Bio) following the manufacturer's protocol. Cells were analyzed by flow cytometry after 24 hours.

Flow Cytometry

Cells in 24 well plates were trypsinized with 40 μL of 0.05% trypsin-EDTA (Gibco) for 1 minute at room temperature, and subsequently resuspended in 100 μL of Hanks' Balanced Salt Solution (HBSS) containing 2.5 mg/ml Bovine Serum Albumin (BSA), 1 mM ethylenediaminetetraacetic acid (EDTA), and 4 units/ml DNase I (NEB). Cells were then filtered through a 40 μm cell strainer (Falcon™) or a 96-well plate cell strainer (Millipore) and analyzed by flow cytometry (CytoFLEX, Beckman Coulter).

Fluorescent Signal Quantification

Flow cytometry data was processed using the Cytoflow python package. Events collected from flow cytometry experiments were first gated based on forward vs. side scatter to select for cells, followed by gating based on scatter parameters, forward area vs forward height, to select for single cells. Data were then gated on fluorescence of the blue fluorescent protein (BFP), emission 450/45, co-transfection marker between 98 and 99.5 percentiles. Median values were taken from mCitrine, 525/40, or mCherry, 610/20, output signals.

Calculation of Protease Activities

Because the HEK1012 cell line is constitutively expressing protease repressible fluorescent proteins, normalized protease activities were calculated using (N−O)/(N−P), where N is observed fluorescence when cells were transfected with the negative control plasmids (protease halves), P is observed fluorescence when cells were transfected with the positive control plasmids (protease halves fused to DHD domains), and O is observed fluorescence in each experiment.

Deterministic Simulation of the Winner-Take-all Neural Network

Numerical simulations were performed in Python to gain insights for the behavior of the circuit. Four types of interactions were modeled: protein synthesis, protein binding, protease cleavage, and first-order protein degradation. Here the list of possible chemical reactions is presented in the order in which they occur (FIG. 2A).

Protein synthesis: Xᵢ, Nᵢⱼ^{D}, Cₖ denote the DHD inputs, N-half proteases, and C-half proteases, respectively. In the 2-input, 2-output system, they comprise 8 species. The superscript D denotes an attached degron (from DHFR). The subscripts i and j denote the identity of the DHD and protease halves, respectively. Ø→Xᵢ; Ø→Nᵢⱼ^{D}; Ø→Cₖ

Protein binding I: Here the DHD, N-half, and C-half protease bind cooperatively to reconstitute the protease complex CₖXᵢNᵢⱼ^{D} (FIG. 2B). These complexes are active only when j=k, otherwise they represent inactive hybrids, containing mismatched halves of different proteases.

Xᵢ+Nᵢⱼ^{D}→XᵢNᵢⱼ^{D}

Cₖ+XᵢNᵢⱼ^{D}→CₖXᵢNᵢⱼ^{D}

Protease cleavage (i), self-activation: active proteases remove the DHFR tags off proteases of the same kind. CₖXᵢNᵢⱼ is a reconstituted protease without the DHFR degradation domain. It is an active protease when j=k. Otherwise, it represents an inactive hybrid containing protease halves from different proteases. Nᵢⱼ is the N-half protease without the DHFR degradation domain.

Proteases Cleave Trimers:

CₖXᵢNᵢⱼ^{D}+CₙXₗNₗₘ^{D}→CₖXᵢNᵢⱼ^{D}+CₙXₗNₗₘ(j=k=m)

CₖXᵢNᵢⱼ+CₙXₗNₗₘ^{D}→CₖXᵢNᵢⱼ+CₙXₗNₗₘ(j=k=m)

Proteases Cleave Dimers:

CₖXᵢNᵢⱼ^{D}+XₗNₗₘ^{D}→CₖXᵢNᵢⱼ^{D}+XₗNₗₘ(j=k=m)

CₖXᵢNᵢⱼ+XₗNₗₘ^{D}→CₖXᵢNᵢⱼ+XₗNₗₘ(j=k=m)

Proteases Cleave Monomers:

CₖXⱼNᵢⱼ^{D}+Nₗₘ^{D}→CₖXᵢNᵢⱼ^{D}+Nₗₘ(j=k=m)

CₖXᵢNᵢⱼ+Nₗₘ^{D}→CₖXᵢNᵢⱼ+Nₗₘ(j=k=m)

Protease cleavage (ii), mutual inhibition: active proteases cleave the C-half protease of different kinds off their DHD domains. Here Cn E is a DHD domain without an attached C-half protease (E for empty). Cₙ^{E}XₗNₗₘ^{D} is a protease complex with the DHFR tag and without the C-half protease. Cₙ^{E}XₗNₗₘ is a protease complex without the DHFR tag and without the C-half protease.

Proteases Cleave Monomers:

CₖXᵢNᵢⱼ^{D}+Cₙ→CₖXᵢNᵢⱼ^{D}+Cₙ^{E}(j=k≠n)

CₖXᵢNᵢⱼ+Cₙ→CₖXᵢNᵢⱼ^{D}+Cₙ^{E}(j=k≠n)

Proteases Cleave Trimers:

CₖXᵢNᵢⱼ^{D}+CₙXₗNₗₘ^{D}→CₖXᵢNᵢⱼ^{D}+Cₙ^{E}XₗNₗₘ^{D}(j=k≠n)

CₖXᵢNᵢⱼ+CₙXₗNₗₘ^{D}→CₖXᵢNᵢⱼ+Cₙ^{E}XₗNₗₘ^{D}(j=k≠n)

Proteases Cleave Trimers:

CₖXᵢNᵢⱼ^{D}+CₙXₗNₗₘ→CₖXᵢNᵢⱼ^{D}+Cₙ^{E}XₗNₗₘ(j=k≠n)

CₖXᵢNᵢⱼ+CₙXₗNₗₘ→CₖXᵢNᵢⱼ+Cₙ^{E}XₗNₗₘ(j=k≠n)

Protein binding (ii): newly formed proteins such as Nᵢⱼ and Cₖ^{E} can now participate in binding. Cₖ^{E}XᵢNᵢⱼ^{D} is a protease complex with the DHFR tag and without the C-half protease. Cₖ^{E}XᵢNᵢⱼ is a protease complex without the DHFR tag and without the C-half protease. Both complexes are inactive due to the missing C-half protease. In contrast, CₖXᵢNᵢⱼ is an active protease when j=k.

Xᵢ+Nᵢⱼ→XᵢNᵢⱼ

XᵢNᵢⱼ+Cₖ→CₖXᵢNᵢⱼ

XᵢNᵢⱼ+Cₖ^{E}→Cₖ^{E}XᵢNᵢⱼ

XᵢNᵢⱼ^{D}+Cₖ^{E}→Cₖ^{E}XᵢNᵢⱼ^{D}

Protease cleavage (iii): Cₙ^{E}XₗNₗₘ^{D} is a new substrate for protease cleavage.

CₖXᵢNᵢⱼ^{D}+Cₙ^{E}XₗNₗₘ^{D}→CₖXᵢNᵢⱼ^{D}+Cₙ^{E}XₗNₗₘ(j=k=m)

CₖXᵢNᵢⱼ+Cₙ^{E}XₗNₗₘ^{D}→CₖXᵢNᵢⱼ+Cₙ^{E}XₗNₗₘ(j=k=m)

Protein degradation: every species with a superscript D, indicating it contains a degron, is assumed to undergo faster degradation than the corresponding species without the superscript D (lacking the degron).

Given the large number of chemical reactions in the system, a Python script was written that automatically and programmably generates these reactions and their corresponding ordinary differential equations (ODEs), using reaction rates provided in Table 5. This system of ODEs was then solved using the odeint solver in Python.

Stochastic Simulation of the Winner-Take-all Neural Network

To perform stochastic simulations using the Gillespie algorithm, mass action rate constants (k) was first converted to stochastic rate constants (c) using the following formulae:

c=N_{A}*V*k 0th order reactions:

c=k 1st order reactions:

c=k/(N_{A}*V) 2nd order reactions:

where N_{A} is the Avogadro constant and V is the cell volume. The resulting list of chemical reactions were simulated in Julia.

TABLE 5
Reaction rates used in simulations

| Rates | Description | Value | | Reference |
|---|---|---|---|---|
| kon₁ | on rate for the first step of | 10⁵ | s⁻¹ M⁻¹ | Chen et al. (Science, 2020) |
| | cooperative DHD binding | | | |
| koff₁ | off rate for the first step of | 100 | s⁻¹ | Chen et al. (Science, 2020) |
| | cooperative DHD binding | | | |
| kon₂ | on rate for the second step of | 10⁵ | s⁻¹ M⁻¹ | Chen et al. (Science, 2020) |
| | cooperative DHD binding | | | |
| koff₂ | off rate for the second step of | 10⁻⁴ | s⁻¹ | Chen et al. (Science, 2020) |
| | cooperative DHD binding | | | |
| konₚ | on rate between proteases and | 10⁵ | s⁻¹ M⁻¹ | estimated |
| | substrates | | | |
| koffₚ | off rate between proteases and | 10⁻⁴ | s⁻¹ | estimated |
| | substrates | | | |
| deg_{reg} | regular protein degradation rate | 10⁻⁵ | s⁻¹ | M. K. Doherty, D. E. Hammond, |
| | | | | M. J. Clague and Gaskell, (J. |
| | | | | Proteome Res., 2009) |
| deg_{DHFR} | degradation rate for DHFR- | 10⁻³ s⁻¹-10⁻² s⁻¹ | | Peth et al. (J. Biol. Chem., 2013) |
| | tagged proteins | | | |
| k_{cat} | protease turnover number | 0.16 | s⁻¹ | Tozser et al. (FEBS J., 2005) |
| V_{cell} | mammalian cell volume | 4*10⁻¹⁵ | L | Luby-Phelps (Int. Rev. Cytol., |
| | | | | 2000) |
| k_{syn} | protein synthesis rate | 0.1-10 | s⁻¹ | estimated |

TABLE 6
Plasmids and mRNAs used in transfection experiments

| Figure panel | Plasmids/mRNAs used |
|---|---|
| 3C | 1. DP_{N1}, 50 ng; DP_{C1}, 50 ng |
| | 2. N₁₁^{D}, 50 ng; C₁ 50 ng |
| | 3. N₁₁^{D}, 50 ng; C₁ 50 ng; X₁, 400 ng |
| | 4. X₁, 50 ng; T₁, 50 ng; C₁, 50 ng |
| | 5. X₁, 50 ng; (N₁₁^{D} + C₁), 50 ng; (N₁₂^{D} + C₂), 450 ng |
| | 6. DP_{N1}, 50 ng; DP_{C1}, 50 ng |
| 3D | mRNAs |
| | X₁, 200 ng; DP_{C1} 200 ng; DP_{C2} 200 ng |
| | N₁₁ and N₁₂ are varied based on Table 7. |
| 3E | (N₁₁^{D} + C₁), 50 ng; (N₂₁^{D} + C₁), 3.3 ng; |
| | (N₁₂^{D} + C₂), 5 ng; (N₂₂^{D} + C₂), 33 ng; |
| | Amount of input proteins are indicated in the figure |
| 3F | (N₁₁^{D} + C₁), 50 ng; (N₂₁^{D} + C₁), 50 ng; |
| | (N₁₂^{D} + C₂), 16 ng; (N₂₂^{D} + C₂), 16 ng; |
| | Amount of input proteins are indicated in the figure |
| 3G | 5:1 -- (N₁₁^{D} + C₁), 50 ng; (N₂₂^{D} + C₂), 10 ng |
| | 2:1 -- (N₁₁^{D} + C₁), 50 ng; (N₂₂^{D} + C₂), 25 ng |
| | 1.5:1 -- (N₁₁^{D} + C₁), 50 ng; (N₂₂^{D} + C₂), 33 ng |
| | 1:1 -- (N₁₁^{D} + C₁), 50 ng; (N₂₂^{D} + C₂), 50 ng |
| | 1:1.5 -- (N₁₁^{D} + C₁), 33 ng; (N₂₂^{D} + C₂), 50 ng |
| | 1:2 -- (N₁₁^{D} + C₁), 25 ng; (N₂₂^{D} + C₂), 50 ng |
| | 1:5 -- (N₁₁^{D} + C₁), 10 ng; (N₂₂^{D} + C₂), 50 ng |
| | Amount of input proteins are indicated in the figure |
| 7B | 7. DP_{N1}, 50 ng; DP_{C1}, 50 ng |
| | 8. DP_{N2}, 50 ng; DP_{C2}, 50 ng |
| 7C | 1. P_{N2}, 50 ng; P_{C2}, 50 ng |
| | 2. N₂₂^{D}, 50 ng; C₂ 50 ng |
| | 3. N₂₂^{D}, 50 ng; C₂ 50 ng; X₂, 400 ng |
| | 4. DP_{N2}, 50 ng; DP_{C2}, 50 ng |
| 7D-7F | FIG. 7D: |
| | 1. P_{N2}, 50 ng; P_{C2}, 50 ng |
| | 2. N₁₂^{D}, 50 ng; C₂ 50 ng |
| | 3. N₁₂^{D}, 50 ng; C₂ 50 ng; X₁, 400 ng |
| | 4. DP_{N2}, 50 ng; DP_{C2}, 50 ng |
| | FIG. 7E: |
| | 9. P_{N1}, 50 ng; P_{C1}, 50 ng |
| | 10. N₂₁^{D}, 50 ng; C₁ 50 ng |
| | 11. N₂₁^{D}, 50 ng; C₁ 50 ng; X₂, 400 ng |
| | 12. DP_{N1}, 50 ng; DP_{C1}, 50 ng |
| | FIG. 7F: |
| | 5. P_{N2}, 50 ng; P_{C2}, 50 ng |
| | 6. N₂₂^{D}, 50 ng; C₂ 50 ng |
| | 7. N₂₂^{D}, 50 ng; C₂ 50 ng; X₂, 400 ng |
| | 8. DP_{N2}, 50 ng; DP_{C2}, 50 ng |
| 7G | 1. P_{N2}, 50 ng; P_{C2}, 50 ng |
| | 2. X₁, 50 ng; T₂, 50 ng; C₂, 50 ng |
| | 3. X₁, 50 ng; (N₁₂^{D} + C₂), 50 ng; (N₁₁^{D} + C₁), 450 ng |
| | 4. DP_{N2}, 50 ng; DP_{C2}, 50 ng |

TABLE 7
N₁₁ and N₁₂

| N₁₁ (ng) | N₁₂ (ng) |
|---|---|
| 500 | 0 |
| 450 | 50 |
| 400 | 100 |
| 300 | 200 |
| 250 | 250 |
| 200 | 300 |
| 100 | 400 |
| 50 | 450 |
| 0 | 500 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A synthetic protein circuit, comprising:
a first n-node protein comprising a first part of a first protease domain, a first dimerization domain, and a second dimerization domain;
a companion first n-node protein comprising a first part of a first protease domain, a third dimerization domain, and a fourth dimerization domain;
a first c-node protein comprising a second part of a first protease domain and a fifth dimerization domain;
a second n-node protein comprising a first part of a second protease domain, a sixth dimerization domain, and a seventh dimerization domain;
a companion second n-node protein comprising a first part of a second protease domain, an eighth dimerization domain, and a ninth dimerization domain;
a second c-node protein comprising a second part of a second protease domain and a tenth dimerization domain;
a first input protein comprising an eleventh dimerization domain; and
a second input protein comprising a twelfth dimerization domain.

2. The synthetic protein circuit of claim 1,
the first dimerization domain, the third dimerization domain, the sixth dimerization domain, and/or the eighth dimerization domain, are the same or have at least about 80% sequence identity;
the second dimerization domain and the seventh dimerization domain are the same or have at least about 80% sequence identity;
the fourth dimerization domain and the ninth dimerization domain are the same or have at least about 80% sequence identity; and/or
the fifth dimerization domain and the tenth dimerization domain are the same or have at least about 80% sequence identity.

3. The synthetic protein circuit of claim 1, wherein:
(a) one or more the first dimerization domain, the third dimerization domain, the sixth dimerization domain, and the eighth dimerization domain are capable of binding one or more of the second dimerization domain, the seventh dimerization, the fourth dimerization domain, the ninth dimerization domain, the fifth dimerization domain, and the tenth dimerization domain;
(b) the eleventh dimerization domain is capable of binding the second dimerization domain and/or the seventh dimerization domain; and/or
(c) the twelfth dimerization domain is capable of binding the fourth dimerization domain and/or the ninth dimerization domain.

4. The synthetic protein circuit of claim 1,
wherein intramolecular binding between the first dimerization domain and the second dimerization domain of the first n-node protein is capable of preventing the first n-node protein from binding the first c-node protein to form a first complex;
wherein intramolecular binding between the first dimerization domain and the second dimerization domain of the first n-node protein is capable of preventing the first n-node protein from binding the second c-node protein to form a second complex;
wherein intramolecular binding between the third dimerization domain and the fourth dimerization domain of the companion first n-node protein is capable of preventing the companion first n-node protein from binding the first c-node protein to form a third complex;
wherein intramolecular binding between the third dimerization domain and the fourth dimerization domain of the companion first n-node protein is capable of preventing the companion first n-node protein from binding the second c-node protein to form a fourth complex;
wherein intramolecular binding between the sixth dimerization domain and the seventh dimerization domain of the second n-node protein is capable of preventing the second n-node protein from binding the first c-node protein to form a fifth complex;
wherein intramolecular binding between the sixth dimerization domain and the seventh dimerization domain of the second n-node protein is capable of preventing the second n-node protein from binding the second c-node protein to form a sixth complex;
wherein intramolecular binding between the eighth dimerization domain and the ninth dimerization domain of the companion second n-node protein is capable of preventing the companion second n-node protein from binding the first c-node protein to form a seventh complex; and/or
wherein intramolecular binding between the eighth dimerization domain and the ninth dimerization domain of the companion second n-node protein is capable of preventing the companion second n-node protein from binding the second c-node protein to form an eighth complex.

5. The synthetic protein circuit of claim 1,
wherein the eleventh dimerization domain of the first input protein is capable of disrupting intramolecular binding between the first dimerization domain and the second dimerization domain of the first n-node protein;
wherein the twelfth dimerization domain of the second input protein is capable of disrupting intramolecular binding between the third dimerization domain and the fourth dimerization domain of the companion first n-node protein;
wherein the eleventh dimerization domain of the first input protein is capable of disrupting intramolecular binding between the sixth dimerization domain and the seventh dimerization domain of the second n-node protein; and/or
wherein the twelfth dimerization domain of the second input protein is capable of disrupting intramolecular binding between the eighth dimerization domain and the ninth dimerization domain of the companion second n-node protein.

6. The synthetic protein circuit of claim 1,
wherein intermolecular binding between the eleventh dimerization domain of the first input protein and the second dimerization domain of the first n-node protein enables the first dimerization domain of the first n-node protein to bind the fifth dimerization domain of the first c-node protein and thereby form a first complex;
wherein intermolecular binding between the eleventh dimerization domain of the first input protein and the second dimerization domain of the first n-node protein enables the first dimerization domain of the first n-node protein to bind the tenth dimerization domain of the second c-node protein and thereby form a second complex;
wherein intermolecular binding between the twelfth dimerization domain of the second input protein and the fourth dimerization domain of the companion first n-node protein enables the third dimerization domain of the companion first n-node protein to bind the fifth dimerization domain of the first c-node protein and thereby form a third complex;
wherein intermolecular binding between the twelfth dimerization domain of the second input protein and the fourth dimerization domain of the companion first n-node protein enables the third dimerization domain of the companion first n-node protein to bind the tenth dimerization domain of the second c-node protein and thereby form a fourth complex;
wherein intermolecular binding between the eleventh dimerization domain of the first input protein and the seventh dimerization domain of the second n-node protein enables sixth dimerization domain of the second n-node protein to bind the fifth dimerization domain of the first c-node protein and thereby form a fifth complex;
wherein intermolecular binding between the eleventh dimerization domain of the first input protein and the seventh dimerization domain of the second n-node protein enables sixth dimerization domain of the second n-node protein to bind the tenth dimerization domain of the second c-node protein and thereby form a sixth complex;
wherein intermolecular binding between the twelfth dimerization domain of the second input protein and the ninth dimerization domain of the companion second n-node protein enables the eighth dimerization domain of the companion second n-node protein to bind the fifth dimerization domain of the first c-node protein and thereby form a seventh complex; and/or
wherein intermolecular binding between the twelfth dimerization domain of the second input protein and the ninth dimerization domain of the companion second n-node protein enables the eighth dimerization domain of the companion second n-node protein to bind the tenth dimerization domain of the second c-node protein and thereby form an eighth complex.

7. The synthetic protein circuit of claim 6,
wherein the first complex and/or the third complex comprise a first protease capable of being in a first protease active state;
wherein the sixth complex and/or the eighth complex comprise a second protease capable of being in a second protease active state; and/or
wherein the second complex, fourth complex, fifth complex, and/or seventh complex do not comprise a first protease capable of being in a first protease active state or a second protease capable of being in a second protease active state.

8. The synthetic protein circuit of claim 1, wherein the first part of the first protease domain and the second part of the first protease domain are capable of associating with each other to constitute a first protease capable of being in a first protease active state when: (i) the first n-node protein binds the first c-node protein to form a first complex; and/or (ii) the companion first n-node protein binds the first c-node protein to form a third complex.

9. The synthetic protein circuit of claim 1, wherein the first part of the second protease domain and the second part of the second protease domain are capable of associating with each other to constitute a second protease capable of being in a second protease active state when: (i) the second n-node protein binds the second c-node protein to form a sixth complex; and/or (ii) the companion second n-node protein binds the second c-node protein to form an eighth complex.

10. The synthetic protein circuit of claim 1, wherein the first n-node protein, the companion first n-node protein, the second n-node protein, and/or the companion second n-node protein comprise a degradation domain, and wherein the presence of said degradation domain causes the protein to be a destabilized state.

11. The synthetic protein circuit of claim 10, wherein:
(a) the first n-node protein and/or the companion first n-node protein comprise a first cut site the first protease in the first protease active state is capable of cutting, wherein the cleavage of said first cut site is capable of inactivating or removing the degradation domain, thereby causing the protein to be in stabilized state;
(b) the second n-node protein and/or the companion second n-node protein comprise a second cut site the second protease in the second protease active state is capable of cutting, and wherein the cleavage of said second cut site is capable of inactivating or removing the degradation domain, thereby causing the protein to be in stabilized state;
(c) the first c-node protein comprises a second cut site the second protease in the second protease active state is capable of cutting, wherein the second cut site is situated between the second part of a first protease domain and the fifth dimerization domain, and wherein cleavage of the said second cut site prevents said first c-node protein from constituting a complex with active protease activity; and/or
(d) the second c-node protein comprises a first cut site the first protease in the first protease active state is capable of cutting, wherein the first cut site is situated between the second part of a second protease domain and the tenth dimerization domain, and wherein cleavage of the said first cut site prevents said second c-node protein from constituting a complex with active protease activity.

12. The synthetic protein circuit of claim 11,
wherein first complexes and/or third complexes are capable of self-activation via first protease-mediated cleavage of the first cut site of first complexes and/or third complexes;
wherein sixth complexes and/or eighth complexes are capable of self-activation via second protease-mediated cleavage of the second cut site of sixth complexes and/or eighth complexes;
wherein first complexes and/or third complexes are capable of mutual inhibition via first protease-mediated cleavage of the first cut site of sixth complexes and/or eighth complexes; and/or
wherein sixth complexes and/or eighth complexes are capable of mutual inhibition via second protease-mediated cleavage of the first cut site of first complexes and/or third complexes.

13. The synthetic protein circuit of claim 1, wherein the degradation domain comprises a degron, and wherein the degron comprises an N-degron, a dihydrofolate reductase (DHFR) degron, a FKB protein (FKBP) degron, derivatives thereof, or any combination thereof.

14. The synthetic protein circuit of claim 1, wherein the first protease and/or the second protease comprises tobacco etch virus (TEV) protease, tobacco vein mottling virus (TVMV) protease, hepatitis C virus protease (HCVP), derivatives thereof, or any combination thereof.

15. The synthetic protein circuit of claim 1, wherein one or more of the first dimerization domain, second dimerization domain, third dimerization domain, fourth dimerization domain, fifth dimerization domain, sixth dimerization domain, seventh dimerization domain, eighth dimerization domain, tenth dimerization domain, eleventh dimerization domain, and twelfth dimerization domain is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

16. The synthetic protein circuit of claim 1, wherein the output of the synthetic protein circuit is either a first protease in a first protease active state or a second protease in a second protease active state.

17. The synthetic protein circuit of claim 16, wherein the output of the synthetic protein circuit depends on (i) the relative levels of the first input protein and the second input protein and (ii) the tunable decision boundaries of the synthetic protein circuit.

18. The synthetic protein circuit of claim 17, wherein the relative levels of the first input protein and/or the second input protein are capable of being regulated via one or more of expression, localization, and stability, and wherein the tunable decision boundaries are capable of being tuned by adjusting the relative levels of the first n-node protein, the companion first n-node protein, the first c-node protein, the second n-node protein, the companion second n-node protein, and/or the second c-node protein.

19. A nucleic acid composition, comprising:
one or more polynucleotides encoding the synthetic protein circuit of claim 1.

20. A method of treating or preventing a disease or disorder in a subject in need thereof, comprising:
expressing the synthetic protein circuit of claim 1 in a cell of a subject in need thereof.


SEQUENCE LISTING (25 sequences)

SEQ ID NO: 1 - protein, 78 aa, synthetic construct
TREELLRENIELAKEHIEIMREILELLQKMEELLEKARGADEDVAKTIKELLRRLKEIIERNQRIAKEHEYIARERSS

SEQ ID NO: 2 - protein, 78 aa, synthetic construct
GSDAYDLDRIVKEHRRLVEEQRELVEELEKLVRRQEDHRVDKKESHEILERLERIIRRSTRILTELEKLTDEFERRTR

SEQ ID NO: 3 - protein, 466 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQSGGGSSETVRFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 4 - protein, 465 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGGGSSENLYFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 5 - protein, 464 aa, synthetic construct
DEKDYHRRLIEHLEDLVRRHEELIKRQKKVVEELERRGLDERLRRVVDRFRRSSERWEEVIERFRQVVDKLRKSVEGSEGSGSEGSGSDDKELDKLLDTLEKILQTATKIIDDANKLLEKLRRSERKDPKVVETYVELLKRHEKAVKELLEIAKTHAKKVEGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQSGGGSSETVRFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 6 - protein, 463 aa, synthetic construct
DEKDYHRRLIEHLEDLVRRHEELIKRQKKVVEELERRGLDERLRRVVDRFRRSSERWEEVIERFRQVVDKLRKSVEGSEGSGSEGSGSDDKELDKLLDTLEKILQTATKIIDDANKLLEKLRRSERKDPKVVETYVELLKRHEKAVKELLEIAKTHAKKVEGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGGGSSENLYFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 7 - protein, 292 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQS

SEQ ID NO: 8 - protein, 291 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 9 - protein, 208 aa, synthetic construct
DSDEHLYKLKTFLENLRRHLDRLDKHIKQLRDILSENPEDERVKDVIDLSERSVRIVKTVIKIFEDSVRKKEGSEGSGSEGSENLYFQSGSKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMD

SEQ ID NO: 10 - protein, 210 aa, synthetic construct
DSDEHLYKLKTFLENLRRHLDRLDKHIKQLRDILSENPEDERVKDVIDLSERSVRIVKTVIKIFEDSVRKKEGSEGSGSEGSETVRFQSGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNS

SEQ ID NO: 11 - protein, 119 aa, synthetic construct
SKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQS

SEQ ID NO: 12 - protein, 117 aa, synthetic construct
KSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMD

SEQ ID NO: 13 - protein, 118 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 14 - protein, 118 aa, synthetic construct
KSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 15 - protein, 204 aa, synthetic construct
GSDDKELDKLLDTLEKILQTATKIIDDANKLLEKLRRSERKDPKVVETYVELLKRHEKAVKELLEIAKTHAKKVEGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQS

SEQ ID NO: 16 - protein, 199 aa, synthetic construct
DSDEHLYKLKTFLENLRRHLDRLDKHIKQLRDILSENPEDERVKDVIDLSERSVRIVKTVIKIFEDSVRKKEGSEGSGSEGSKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMD

SEQ ID NO: 17 - protein, 203 aa, synthetic construct
GSDDKELDKLLDTLEKILQTATKIIDDANKLLEKLRRSERKDPKVVETYVELLKRHEKAVKELLEIAKTHAKKVEGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 18 - protein, 200 aa, synthetic construct
DSDEHLYKLKTFLENLRRHLDRLDKHIKQLRDILSENPEDERVKDVIDLSERSVRIVKTVIKIFEDSVRKKEGSEGSGSEGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 19 - protein, 454 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 20 - protein, 454 aa, synthetic construct
GTERKLLERSRRLQEESKRLLDEMAEIMRRIKKLLKKARGADEKVLDELRKIIERIRELLDRSRKIHERSEEIAYKEEGSEGSGSEGSGSDDKELDKLLDTLEKILQTATKIIDDANKLLEKLRRSERKDPKVVETYVELLKRHEKAVKELLEIAKTHAKKVEGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGGGSSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 21 - protein, 236 aa, synthetic construct
VSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 22 - protein, 238 aa, synthetic construct
GGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKS

SEQ ID NO: 23 - protein, 239 aa, synthetic construct
VSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKS

SEQ ID NO: 24 - protein, 261 aa, synthetic construct
ETVRFQYHKSGAWKLPVSLVKGGTSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKS

SEQ ID NO: 25 - protein, 264 aa, synthetic construct
ENLYFQYHKSGAWKLPVSLVKGGGSVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKS